(19) United States
(12) Patent Application Publication
(10) Pub. No.: US 2024/0418707 A1
(43) Pub. Date: Dec. 19, 2024

(54) SELF-ORGANIZING NEURO-MUSCULAR JUNCTION CELL CULTURE MODEL

(71) Applicant: Max-Delbrück-Centrum für Molekulare Medizin in der Helmholtz-Gemeinschaft, Berlin (DE)

(72) Inventors: Asimina GKOUTI, Berlin (DE); Alessia URZI, Norderstedt (DE)

(21) Appl. No.: 18/335,765

(22) Filed: Jun. 15, 2023

(51) Int. Cl. G01N 33/50; C12N 5/071

(52) U.S. Cl. CPC G01N 33/5082; C12N 5/0697; C12N 2501/105; C12N 2501/115; C12N 2501/12; C12N 2501/415; C12N 2506/45

(57) ABSTRACT

An in vitro method of generating a neuro-muscular junction cell culture model. In other aspects the invention relates to an in vitro cell culture model, including segregated neural and mesodermal progenitor cells, or comprising segregated partially differentiated neural and myoblast cells, or comprising differentiated brachial, cervical, thoracic and/or lumbar motor neurons, forming neuro-muscular junctions. Also disclosed is the use of the models and methods in screening methods and/or for studying disease.

The invention is the field of cell biology, cell culture models and corresponding screening methods.

The invention relates to an in vitro method of generating a neuro-muscular junction cell culture model. In other aspects the invention relates to an in vitro cell culture model, comprising segregated neural and mesodermal progenitor cells, or comprising segregated partially differentiated neural and myoblast cells, or comprising differentiated brachial, cervical, thoracic and/or lumbar motor neurons, forming neuro-muscular junctions. The invention relates further to the use of said models and methods in screening methods and/or for studying disease.

### BACKGROUND

Neuromuscular diseases embrace a wide range of pathologies such as (i) Motor-Neuron Diseases (MNDs) like amyotrophic lateral sclerosis (ALS) and spinal muscular atrophy (SMA), (ii) specific myopathies such as Becker's and Duchenne's muscular dystrophies and (iii) auto-immune neuromuscular diseases such as myasthenia gravis. Neuromuscular diseases are caused by functional defects of the nervous system or skeletal muscle or arise by defects of the neuromuscular junction (NMJ). The mechanisms underlying the specificity of neuromuscular diseases remain largely unresolved. This is in part due to the inability to obtain sufficient quantities of specific types of spinal cord neurons and skeletal muscle cells to perform mechanistic studies.

Human pluripotent stem cells (hPSCs) have been used extensively to generate reductionist models of either spinal cord motor neurons or skeletal muscles to study neuromuscular system disorders¹⁻⁷. While these models are very useful for studying cell-autonomous effects, they do not reflect the complex interactions of diverse cell types such as spinal cord neurons, skeletal muscles, glia and terminal Schwann cells required to study neuromuscular junction (NMJ).

During the development of the nervous system many different types of neurons must differentiate into their correct positions in a complex pattern. With respect to the head-to-tail axes of the neural tube, spinal motoneurons diversify to form columns at specific positions. This columnar organization is linked to the formation of correct axon tracts and neuronal connections. Therein lateral motor column neurons that innervate the limbs are only present at the brachial (forelimb) and lumbar (hindlimb) levels, while column of Terni (CT) neurons that project to the sympathetic ganglia, are present at the thoracic level. The patterned expression of Hox proteins, e.g., Hox-C, in response to FGFs are considered a key component of the development of lateral motor column motor neurons that form alongside the developing limbs (Guthrie, Current Biology, 2004).

Bioengineering and co-culture approaches have increased in complexity and have been successfully used as alternatives to reductionist models⁸⁻¹⁴. However, co-culture approaches are limited by the necessity to derive the required cell types separately before combining them in a single culture. This imposes practical limitations but also leads to compromises in the choice of the common culture medium conditions, which in turn limits the long-term maintenance of the co-cultures. In the prior art, Mazaleyrat et al. and WO2021233810A1 have developed a method for differentiating muscle cells and motor neurons in culture, but it does not employ intrinsic self-assembly, simultaneous co-differentiation or segregation of progenitors at day 6 (NPs+PSMs). For differentiation in the method of Mazaleyrat et al., inhibition of the BMP pathway is performed from the first day of cultivation and neuronal differentiation is induced consecutively.

The potential of neuromesodermal progenitor (NMP) cells to generate the different cell types required for functional NMJs has opened up new opportunities for investigating NMJ disorders¹⁷. However, no study has thus far succeeded in simultaneously generating position specific NMJ models in adherent culture conditions.

Hence, in light of the prior art there remains a significant need in the art to provide advanced two-dimensional cell culture models that facilitate the simultaneous generation of neuronal spinal cord and skeletal muscle tissue comprising functional neuromuscular junctions. There also remains a need for an NMJ model that facilitates long-term maintenance of neuromuscular cultures, which has not been possible with previously established co-culture models.

### SUMMARY

In light of the prior art, a technical problem underlying the invention was the provision of an alternative or improved cell culture model comprising functional neuromuscular junctions. Another problem underlying the invention was to obtain segregation of neural and mesodermal progenitor cells in in vitro culture conditions using a single cell culture system (avoiding co-culture). Another problem underlying the invention was to obtain a cell culture model comprising differentiated muscle cells with innervating motor neurons, forming neuromuscular junctions, in in vitro culture conditions using a single cell culture system (avoiding co-culture).

The above-mentioned problem is solved by the features of the independent claims. Preferred embodiments of the present invention are provided by the dependent claims.

The invention therefore relates to an in vitro method of generating a neuro-muscular junction cell culture model, comprising:
a) cultivating a first cell culture comprising pluripotent stem cells (PSCs), for at least 3 days, in a first culture medium comprising:
   a WNT pathway-activator, and
   FGF, preferably basic FGF (FGF2),
b) replacing the first culture medium by a second culture medium, comprising:
   a BMP pathway inhibitor, and
      (i) a TGFβ pathway inhibitor, or
      (ii) GDF11,
         Sonic Hedgehog (SHH) smoothened agonist (SAG) and/or retinoic acid (RA),
         a WNT pathway-activator, and
         FGF, HGF and/or IGF1, and
   cultivating the cells for at least 3 days,
   thereby generating a cell culture model comprising segregated neural and mesodermal progenitor cells, and/or differentiated muscle cells with innervating motor neurons, forming neuromuscular junctions.

In embodiments, the cell culture is first exposed to a BMP pathway inhibitor, and (i) a TGFβ pathway inhibitor or (ii) GDF11, according to the second culture medium in step b), after 3 days of culture in the first cell culture medium. In embodiments, these components may be understood as dual SMAD inhibitors.

The invention therefore employs a specific (in comparison to the prior art, delayed) administration of a BMP receptor, and (i) a TGFβ pathway inhibitor or (ii) GDF11, in order to achieve the models described herein. In embodiments, the administration of the second cell culture medium in step b) facilitates a first exposure of the cell culture to said dual SMAD inhibitors, in particular to a BMP receptor, and (i) a TGFβ pathway inhibitor or (ii) GDF11.

The inventors therefore describe the development of a robust and efficient neuromuscular junction model from human pluripotent stem cells that can be maintained long-term in simple adherent conditions. The timely application of specific patterning signals, in particular based on the first exposure of the cell culture to (dual) SMAD inhibitors after 3 days in culture, instructs the simultaneous development and differentiation of segregated neural and mesodermal progenitor cells, and/or differentiated muscle cells with innervating motor neurons, forming neuromuscular junctions. The model therefore enables a model with position-specific spinal neurons, such as brachial spinal neurons, with skeletal muscles, and/or terminal Schwann cells.

In contrast, the prior art method of Mazaleyrat et al. applies inhibition of the BMP/SMAD pathway from the first day of cultivation. However, as shown by the inventors in the examples herein (see e.g., FIG. 1), the step of immediate SMAD-pathway inhibition from day 0 onwards (e.g., according to the method of Mazaleyrat et al.) excludes co-differentiation of both neuronal and myogenic progenitors from an early stage, as shown with the present invention. The inventors have shown in the examples that early treatment of hPSCs with a SMAD inhibitor, even in the presence of WNT/bFGF, primes NMPs towards an exclusive neural identity. Hence, the Mazaleyrat et al. method is only able to induce in a first step muscle differentiation and subsequently the differentiation of neurons. On the contrary, the delayed treatment with inhibitors of SMAD signaling after the establishment of the NMP state, e.g., after 3 initial days of culture, according to the present invention allows the simultaneous differentiation to both neural and PSM lineages. This approach of delayed exposure to SMAD pathway inhibition, e.g., by combined BMP receptor and TGFbeta-signaling inhibition, leads to a higher functionality and a greater degree of interaction between the obtained motor neurons and muscle cells.

During embryonic development said tissues develop in parallel (synchronously) and they start interacting/communicating from an early time point. On the contrary, the method of Mazaleyrat et al., which generates the respective cell types consecutively, has an inferior degree of interaction between the neuronal and muscle cells and an inferior functionality of any NMJs. The model generated according to the present model however achieves synchronous development of the neuronal and muscular cell types and achieves a segregation of the same, preferably as early as from day 6 onwards. This segregated model shows, preferably from day 6, distinct development stages and the cell types can start interacting from an early time point, such that the inherent biological processes involved in NMJ development and function are more accurately reflected and embodied by the present cell culture model.

As described in the examples in more detail below, high-content imaging reveals self-organized bundles of aligned muscle fibres surrounded by innervating motor neurons that form functional neuromuscular junctions. Optogenetic activation and pharmacological interventions show that the spinal neurons actively instruct the synchronous skeletal muscle contraction. This model successfully recapitulates the early pathology of spinal muscular atrophy, opening up the opportunity for high throughput studies in disease modelling and drug development.

Further aspects, embodiments and examples of the invention are provided below.

In embodiments, the in-vitro method comprises, after step a) and b), additionally:
c) replacing the at least second culture medium by a culture medium comprising HGF and/or IGF1, and optionally a gamma-secretase inhibitor (e.g., DAPT), and cultivating the cells for at least 4 days,
   and optionally replacing the culture medium by a medium suitable for maintenance of the cell culture model.

In embodiments, these steps also enable generating a cell culture model comprising segregated neural and mesodermal progenitor cells, and/or (terminally) differentiated muscle cells with innervating motor neurons, forming neuromuscular junctions.

In embodiments, in step c) the cell culture model comprises segregated neural and mesodermal progenitor cells and/or (terminally) differentiated muscle cells with innervating motor neurons, forming functional neuromuscular junctions.

In embodiments, the method employs pluripotent stem cells (PSCs) obtained from a patient, for example a patient with a neuromuscular disease. In embodiments where the present model is/was generated from ‘diseased’ pluripotent cells, e.g., derived from a diseased patient or comprising an (engineered/artificially introduced) disease-specific mutation etc., at least a fraction of the formed neuromuscular junctions might be non-functional or dysfunctional or might even be partially or entirely absent. References to a “functional” NMJ refer primarily, but not exclusively, to using “healthy” human pluripotent cells in the method.

In embodiments, diseased cells might also be generated from stem cells comprising artificially introduced or genetically engineered mutations. In embodiments, this approach can also be applied for patient-specific IPS cells to generate a patient-specific model comprising the patient specific genetic background in combination with a certain mutation, e.g., a tissue-specific mutation, that could not be obtained from the patient by biopsy. In other embodiments the present model can be used to study a disease and/or the treatment of a disease in a patient-specific background, e.g., also in case of patient-specific germline mutations, such that developed from patient-specific IPS cells the present model can serve as a personalized NMJ model for a diseased or predisposed patient.

In embodiments, step a) of the method is conducted on or after day 0 of culture. In embodiments, the first cell culture is cultivated in a first culture medium according to step a) for at least 1 day, at least 2 days, at least 3 days, or at least 4, 5, 6, 7, 8, 9, 10, 11, 12, 13, 14 days or even longer, or between 1-10 days, 1-5 days, between 2-4 or 3-5 days or for about 3 days.

In embodiments, step b) of the method is conducted between day 3 and day 10 of culture. In embodiments, the cell culture is cultivated in a second culture medium according to step b) of the method for at least 1 day, at least 2 days, at least 3 days, or at least 4, 5, 6, 7, 8, 9, 10, 11, 12, 13, 14, 15, 16, 17, 18, 19, 20, 21 days or even longer, or between 1-10 days, 3-10 days, 3-7 days, 1-5 days, 2-14 or 1-21 days or for at about 7 days.

All time ranges disclosed herein may, in embodiments, also comprise a variation of ±1 day, of ±2, 3, 4 or 5 days, or ±10 days, or even more variation.

In embodiments, step c) of the method is conducted between day 10 and day 28 of culture. In embodiments the cell culture is cultivated in a (third) culture medium according to step c) of the method for at least 1 day, at least 2 days, at least 3 days, or at least 4, 5, 6, 7, 8, 9, 10, 11, 12, 13, 14, 15, 16, 17, 18, 19, 20, 21, 28, 35, 42, 50, 57, 60, 65, 70, 75, 80, 85, 90, 95, 100 days or even longer, or between 1-200 days, 1-100 days, 1-300 days, 1-50 days, 1-18 or 1-40 days or for at about 40 or 90 days.

To overcome the above-listed limitations of the prior art methods, and while retaining the advantage of simultaneous development, maturation and long-term culture of NMJs, the inventors sought to derive an NMJ model system in 2 dimensions (2D) through an NMP intermediate state.

The inventors surprisingly found that specification of NMP identity followed by timely inhibition of BMP and TGFb signaling instructed the development of self-organizing neural and mesodermal progenitors within a short window of time. The simultaneous generation of the spinal cord and skeletal muscle tissues from NMPs with the present method ensures the early establishment of crucial functional interactions that stimulate their cooperative maturation.

Accordingly, in the present invention, the inventors establish a human neuromuscular junction model from pluripotent stem cells that resembles the embryonic developmental processes. Pluripotent stem cells are first differentiated to neuromesodermal progenitors that are the building blocks of the human neuromuscular system. In embodiments, human PSC-derived neuromesodermal progenitors are then differentiated simultaneously to spinal cord neural progenitors and mesodermal progenitors that self-organize under adherent culture conditions to form functional neuromuscular junctions. By changing the extracellular signals, the inventors were surprisingly able to direct the differentiation of neuromesodermal progenitors to generate a position specific neuromuscular junction model corresponding to the brachial, the cervical/brachial/thoracic (brachial, and optionally cervical and/or thoracic) or the lumbar spinal neuron level.

Unexpectedly, this organization could even be re-established after passaging of cells segregated into spatially organized spinal cord neurons, skeletal muscle and terminal Schwann cells, forming functional NMJs that matured over time. It was entirely surprising that the neuromuscular cultures generated according to the present invention started contracting at day 50 due to functional connectivity between the motor neurons (MNs) and the skeletal muscle fibers.

In addition, it appeared that apart from MNs and skeletal muscles, terminal Schwann cells might be important for the maturation and stability of NMJs. At day 50, S100β+ Schwann cells were present and by day 100, they could be found at the NMJs, located between the AChR clusters and the neural axons. Collectively, these data suggested that, in an exemplary embodiment of the present invention, brachial MNs interacted with the skeletal myofibers to form NMJs supported by terminal Schwann cells.

As shown in the Examples in more detail below, in-depth functional characterization using optogenetic activation of spinal cord neurons and pharmacological manipulation of the NMJs showed that the synchronous contraction of skeletal muscles was instructed by the MNs. Strikingly, spinal cord neurons in the example NMJ model primarily have a brachial identity, and both median and lateral motor column MNs, interneurons and glial cells were present. As outlined in the examples below in detail, in a proof of principle experiment, said soNMJ model was used to recapitulate the disease pathology observed in patients with spinal muscular atrophy (SMA). Surprisingly, SMA-specific neuromuscular cultures developed a severe phenotype that affected the contraction of the skeletal muscle, thus resembling the patient pathology.

As a proof of concept, the inventors have previously shown that hPSC-derived NMPs are sufficient for generating functional NMJs in 3D neuromuscular organoids (NMOs)¹⁷. Neuromuscular organoids reach 2-3 mm in size after 30 days in culture and 4-5 mm after 50 days, but because of their size, they cannot be maintained in the multiwell-format (96-well or smaller) necessary for high-throughput studies.

In general, the complex neuromuscular network that controls body movements is the target of severe diseases that result in paralysis and death. Presently, to the knowledge of the inventors, no approach has been established that facilitates the generation two dimensional (2D) neuromuscular cell culture model that could facilitate long-term maintenance of cultures comprising neuromuscular junctions (NMJ). Previously established co-culture models did not accomplish this, even when chips, scaffolds, or protein matrices were used^{8,9,15,16}.

As is shown in more detail below, the inventors developed a robust and efficient self-organizing neuromuscular junction (soNMJ) model, preferably from human pluripotent stem cells, that can be maintained long-term in simple adherent conditions.

In embodiments, the timely application of specific patterning signals in the context of the present model is capable of inducing the simultaneous development and differentiation of position-specific brachial (or cervical and/or brachial and/or thoracic, or brachial, and optionally cervical and/or thoracic) spinal neurons, skeletal muscles, and terminal Schwann cells. As shown in the examples below, in exemplary embodiments high-content imaging revealed self-organized bundles of aligned muscle fibers surrounded by innervating motor neurons that formed functional neuromuscular junctions. Further, using optogenetic activation and pharmacological interventions the inventors could show that the spinal neurons actively instructed the synchronous skeletal muscle contraction. Therefore, the model according to the invention was shown to successfully recapitulate, e.g., the early pathology of spinal muscular atrophy, opening up the opportunity for high throughput studies in disease modeling and drug development. Thus, model(s) according to the invention are beneficially able to address unmet needs in the neuromuscular disease field.

In embodiments, the invention can be instrumental to study diseases that affect the human neuromuscular system. The present models constitute the first self-organizing 2D position-specific neuromuscular junction models generated from human PSCs through a neuromesodermal progenitor intermediate state. By way of example, the inventors could demonstrate that timely application of appropriate instructive and mitogenic signals, preferably until day 25, generates a neuromuscular model of defined rostrocaudal and columnar identity.

Then, the co-developing cell types provide the necessary autocrine and paracrine signals giving rise to a dynamic, self-instructing model system that develops and matures with minimal requirements for exogenously added differentiation and maturation factors. Timely application of specific extracellular signals results in the generation of brachial (or cervical and/or brachial and/or thoracic) or lumbar specific NMJs. The present models constitute the first models where motor neurons of specific rostrocaudal identity are generated with skeletal muscles that self-organize to form functional NMJs.

The present position-specific NMJ models will open up the exciting opportunity to study the selective vulnerability of position-specific neurons to neuromuscular diseases. The establishment of the present soNMJ model offers an efficient, defined, and robust platform that can be used for mechanistic studies and high throughput approaches. The simplicity and scalability of the present soNMJ model is ideal for drug screening studies and development of novel therapeutic approaches for neuromuscular diseases, paving the road to personalized medicine.

In embodiments, the cell culture model is a two-dimensional (2D), self-organizing neuro-muscular junction cell culture model.

As used herein, a two-dimensional (2D) cell culture model, such as the present neuro-muscular junction cell culture model, is preferably a cell culture that grows in one or more layers, preferably in one layer, more preferably in one (flat) layer of cells, in a cell culture dish, vessel or cell culture plate. Preferably the term two-dimensional (2D) is meant to differentiate the present cell culture model from a three-dimensional (3D) cell culture (model), which grows as an organoid or in multiple (complex) layers. A two-dimensional cell culture model is preferably a layer of cells that can be established, grown and/or maintained in a cell culture dish or plate, preferably in an adherent manner, such as in a Matrigel or Geltrex coated dish or plate, and that can be used, e.g., for high throughput screenings, such as drug or cytotoxicity screenings.

As used herein, the term ‘self-organizing’ preferably describes the capability of the present cell culture model to form bundles of muscle fibers that are surrounded by and/or preferably at least partially interwoven with axonal structures of neural cells while preferably forming neuro-muscular junctions. Said capability of the model is preferably distinct from a mere co-culture of neurons and muscles cells or their precursors, or from a parallel—and (mostly) independent—differentiation of said cells in co-culture. The herein described capability of the present model is preferably induced, maintained and/or guided by the successive addition and removal of specific factors and/or signaling molecules to the cell culture medium of the cell culture and subsequent incubation. Hence, ‘self-organizing’ refers to the property that all the cell types such as e.g., spinal cord neurons, skeletal muscle cells, and/or terminal Schwann cells are generated from pluripotent stem cells and self-organize to form the neuro-muscular junctions upon treatment with the cell culture conditions described herein.

Hence, the term ‘self-organizing’ preferably refers to the surprising capability of the present cell culture model and present method to initiate the formation of bundles of muscle fibers that are surrounded and preferably at least partially interwoven with axonal structures of neural cells while forming neuro-muscular junctions, only by the inventive combination of specific culture conditions, and the addition of specific supplements to the culture media and/or due to their specific duration/time span of incubation. The term ‘self-organizing’ also implies that no additional cells or other cell types are added and that no co-culture of different cell types, e.g., which were independently differentiated, is performed.

The present method in its herein described embodiments constitutes a new and inventive procedure/workflow of successive addition and removal of specific factors and/or signaling molecules to the cell culture medium of initially pluripotent stem cells, which enables the generation of the two-dimensional (2D), self-organizing neuro-muscular junction cell culture model according to the invention.

Said two-dimensional (2D), self-organizing neuro-muscular junction cell culture model can be useful at different stages of cell differentiation, such as after a few days of the present method, e.g., as a model comprising precursors cells or intermediates (e.g., after ˜3, 6, 14, 20 or 25 days), or as a model of differentiated cells (muscle cells and neurons; e.g., after ˜50 or more days) comprising the herein described functional neuro-muscular junctions.

In embodiments of the in-vitro method according to the invention, step b) further comprises replacing after 2-5 days the second culture medium by a third culture medium comprising retinoic acid and/or SAG, and optionally HGF and/or IGF1, and cultivating the cells for 2-5 days.

In embodiments of the in-vitro method according to the invention the method comprises the steps of:
a) Cultivating a first cell culture comprising pluripotent stem cells (PSCs; preferably iPS cells, more preferably iPSCs of a patient), for at least 3 days, in a first culture medium comprising a WNT pathway-activator, and an FGF,
b) replacing the first culture medium by a second culture medium comprising one or more of the group comprising a WNT pathway-activator, an FGF, Sonic Hedgehog (SHH)-smoothened agonist (SAG), a BMP pathway inhibitor, and either (i) a TGFβ pathway inhibitor and retinoic acid (RA), or (ii) GDF11, and cultivating the cells for 2-4 days, preferably for 3 days,
c) replacing the second culture medium by a third culture medium comprising retinoic acid, SAG, HGF and IGF1, and cultivating the cells for 2-5 days, preferably for 4 days, wherein on the first day a Rho-associated protein kinase inhibitor (ROCKi) is added to the culture medium for 6-24 hours,
d) replacing the third culture medium by a fourth culture medium comprising HGF and IGF1, and optionally a gamma-secretase inhibitor, and cultivating the cells for 1-5 days, preferably for 4 days,
e) replacing the fourth culture medium by a fifth culture medium comprising HGF and IGF1, and cultivating the cells for at least 11 days,
f) replacing the fifth culture medium by a sixth culture medium comprising a ROCK inhibitor, ITS (Insulin, Transferrin, Selenium), HGF and IGF1, and cultivating the cells for 1-4 days, preferably for 3 days, and
g) replacing the sixth culture medium by a seventh culture medium supplemented with ITS and cultivating the cells for at least one day.

In embodiments, (i) a WNT pathway-activator (e.g., in the first culture medium) is a WNT protein, a WNT pathway ligand or agonist, a WNT-activator and/or a GSK3 inhibitor.

In embodiments, a first culture medium comprises (i) WNT, a WNT-activator and/or a GSK3 inhibitor, and (ii) an FGF.

In embodiments, the WNT pathway can be activated by an activator of WNT signaling, a WNT activator, a WNT agonist or ligand, a WNT protein and/or by inhibition of factors, proteins or receptors, such as a GSK3, that inhibit WNT-signaling (hence, e.g., by a GSK3-inhibitor).

In embodiments a WNT pathway activator can also be a WNT activator, a WNT pathway ligand or agonist or WNT agonist.

In embodiments, the second culture medium comprises one or more of the group comprising (I) a WNT pathway-activator (e.g., such as WNT, a WNT-activator and/or a GSK3 inhibitor), (II) an FGF, (III) Sonic Hedgehog (SHH)-smoothened agonist (SAG), (IV) a BMP pathway inhibitor, and either (i) a TGFβ pathway inhibitor and retinoic acid (RA), or (ii) GDF11.

In embodiments, a BMP pathway inhibitor may be an inhibitor of a BMP receptor. The term BMP pathway inhibitor may be used interchangeably with an inhibitor of a BMP signaling, an inhibitor of the BMP pathway. In embodiments, a BMP pathway inhibitor may be an inhibitor of SMAD signaling.

In embodiments, a TGFβ pathway inhibitor may be an inhibitor of a transforming growth factor-β receptor (TGF-βR). The term TGFβ pathway inhibitor may be used interchangeably with an inhibitor of TGF-β signaling, an inhibitor of the TGF-β pathway. In embodiments, the TGFβ pathway inhibitor is an inhibitor of SMAD signaling.

In embodiments, said media can be referred to as a neural progenitor and pre-somitic mesoderm (NP+PSM) medium.

In embodiments, a third culture medium comprises (i) retinoic acid (RA), (ii) SAG, (iii) HGF and (iv) IGF1, and optionally (v) a Rho-associated protein kinase inhibitor (ROCKi), wherein the optional ROCKi is preferably only added for a certain duration, e.g., several hours (e.g., between 6-36 hrs.). In embodiments, said media can be referred to as a myoblast induction medium.

In embodiments, a fourth culture medium comprises (i) HGF and (ii) IGF1, and optionally (iii) a gamma-secretase inhibitor. In embodiments, said media can be referred to as motor neuron (MN) medium.

In embodiments, a fifth culture medium comprises (i) HGF and (ii) IGF1. In embodiments, said media can be referred to as motor neuron (MN)+myoblast maintenance medium:

In embodiments, a sixth culture medium comprises (i) ITS (Insulin, Transferrin, Selenium), (ii) HGF and (iii) IGF1 and optionally (iv) a ROCK inhibitor and/or optionally (v) prednisolone. In embodiments, said media can be referred to as MN+SkM (motor neuron+skeletal muscle) medium.

In embodiments, the cell culture medium is supplemented with a glucocorticoid, preferably prednisolone, after 25 day in culture, preferably once every week. This treatment of the present cell culture model is optional, and improves the development/formation of muscle cells and fibers, especially in cultures of ‘diseased’ initial (patient) cells.

In embodiments, a seventh culture medium comprises ITS. In some embodiments, prednisolone or another glucocorticoid is added to the media, preferably only for a certain duration, e.g., for 24-48 hours. In embodiments, said seventh culture medium is a media suited for long-term/maintenance culture of the present model. In embodiments said media can be referred to as MN+SkM (motor neuron+skeletal muscle) maintenance medium.

In embodiments, one or more of the afore mentioned cell culture media is a neurobasal (NB) cell culture medium.

The various cell culture mediums described above may also be applied with different timing. For example, a sixth or seventh cell culture media may also be applied at any other step of the method, should the functional characteristics of the model in essence be maintained.

In embodiments, step a) of the method is conducted on or after day 0 of culture. In embodiments, step a) of the method is conducted between day 0 and day 3 of culture. In embodiments the first cell culture is cultivated in a first culture medium according to step a) of the method for at least 3 days, for at least 1 day, at least 2 days, or at least 4, 5, 6, 7, 8, 9, 10, 11, 12, 13, 14 days or even longer, or 1-10 days, 1-5 days, 2-4 or 3-5 days or for 3 days.

In embodiments, step b) of the method is conducted on or after day 3 of culture. In embodiments, step b) of the method is conducted between day 3 and day 6 of culture. In embodiments the cell culture is cultivated in a second culture medium according to step b) of the method for at least 3 days, for at least 1 day, at least 2 days, or at least 4, 5, 6, 7, 8, 9, 10, 11, 12, 13, 14 days or even longer, or 1-10 days, 1-5 days, 2-4 or 3-5 days or for 3 days.

In embodiments, step c) of the method is conducted on or after day 6 of culture. In embodiments, step c) of the method is conducted between day 6 and day 10 of culture. In embodiments, the cell culture is cultivated in a third culture medium according to step c) of the method for at least 4 days, for at least 1 day, at least 2 days, or at least 3, 5, 6, 7, 8, 9, 10, 11, 12, 13, 14 days or even longer, or 1-10 days, 1-5 days, 2-4 or 3-5 days or for 4 days.

In embodiments, step d) of the method is conducted on or after day 10 of culture. In embodiments, step d) of the method is conducted between day 10 and day 14 of culture. In embodiments, the cell culture is cultivated in a fourth culture medium according to step d) of the method for at least 4 days, for at least 1 day, at least 2 days, or at least 3, 5, 6, 7, 8, 9, 10, 11, 12, 13, 14 days or even longer, or 1-10 days, 1-5 days, 2-4 or 3-5 days or for 4 days.

In embodiments, step e) of the method is conducted on or after day 14 of culture. In embodiments, step e) of the method is conducted between day 14 and day 25 of culture. In embodiments, the cell culture is cultivated in a fifth culture medium according to step e) of the method for at least 6 days, for at least 10 days, for at least 11 days, or at least 1, 2, 3, 4, 5, 6, 7, 8, 9, 10, 11, 12, 13, 14 days or even longer, or 1-14 days, 6-12 days, 5-14 or 7-14 days or for 6 or 11 days.

In embodiments, step f) of the method is conducted on or after day 25 of culture. In embodiments, step f) of the method is conducted between day 25 and day 28 of culture. In embodiments, the cell culture is cultivated in a sixth culture medium according to step f) of the method for at least 4 days, for at least 1 day, at least 2 days, or at least 3, 5, 6, 7, 8, 9, 10, 11, 12, 13, 14 days or even longer, or 1-10 days, 1-5 days, 2-4 or 3-5 days or for 4 days.

In embodiments, step g) of the method is conducted on or after day 28 of culture. In embodiments, step g) of the method is conducted between day 28 and day 50-365 of culture. In embodiments, the cell culture is cultivated in a seventh culture medium according to step g) of the method for at least 22 days, for at least 20 day, at least 30 days, or at least or exactly 1, 2, 3, 4, 5, 6, 7, 8, 9, 10, 11, 12, 13, 14, 21, 22, 28, 30, 35, 40, 45, 50, 55, 60, 65, 70, 75, 80, 85, 90, 95, 100, 150, 200, 250, 300, 350, 365, 400, 450, 500, 550, 600, 650, 700, 730, 750 days or even longer, or for 22-340 days, 1-30 days, 20-300 or 1-600 days or for 22-337 days.

All time ranges disclosed herein may in embodiments also comprise a variation of ±1 day, of ±2, 3, 4 or ±5 days, of ±10 days, or ±14, or ±28, or ±35 days, or even more variation.

One of the advantages of the present method and models is that they allow, for the first time, the generation of position specific self-organizing neuromuscular junction models from human PSCs. Herein it is described for the first time that human PSC derived NMPs exposed to certain combination of signals differentiate to either a brachial (or cervical and/or brachial and/or thoracic) or lumbar specific neuromuscular junction model. Another important advantage of the present model over prior art co-culture models is that it can be maintained for more than 100 days.

Further advantages of the present invention are the generation of self-organizing neuromuscular junctions from human PSCs through an NMP intermediate state, the capability that motor neurons acquire a specific brachial (or cervical and/or brachial and/or thoracic) or lumbar spinal cord identity and generate position specific NMJs depending on the extracellular signals that NMPs receive from day 3-day 6. Moreover, other cell types, including interneurons, glia cells, satellite cells and/or terminal Schwann cells can co-develop in the present soNMJ model. Apart from MNs and skeletal muscles, terminal Schwann cells are in embodiments important for the maturation and stability of NMJs.

Further, functional neuromuscular junctions form in embodiments through self-organization recapitulating the embryonic developmental processes and strikingly skeletal muscles contract in response to motor neurons. In addition, optogenetic activation of motor neurons results in the synchronous contraction of the skeletal muscle. Finally, the present models can be used to study neuromuscular diseases such as amyotrophic lateral sclerosis and spinal muscular atrophy. The invention can also be used to study the mechanisms of disease that affect the human neuromuscular system such as spinal muscular atrophy and/or to perform drug screenings for different diseases that affect the neuromuscular system.

In embodiments, the neuromuscular junctions form at day 50. In embodiments, the neuromuscular junctions form between day 10 and 50.

In embodiments, the cell culture is passaged/re-seeded/re-plated and/or split between day 25 and day 30 at least once. In embodiments, the cell culture is maintained/cultured in a culture medium comprising HGF/IGF after the passaging/re-seeding/re-plating/splitting the cell culture, preferably between day 25 and day 30, at least until day 50. In embodiments the cell culture is maintained/cultured in a culture medium comprising HGF/IGF after day 25-30, e.g., until day 50, 57, 64, 71, 78, 85, 92, 100, 106, 150, 200, 250, 300, 350, 400, 450, 500 or even longer.

In embodiments, a WNT-pathway activator, such as, e.g., the protein WNT, a WNT-activator, or a GSK3 inhibitor can be used herein alternatively or in combination with each other, such as a combination (cocktail) of two or more thereof. In embodiments, WNT protein may be added to the cell culture media, in some preferred embodiments the WNT protein is a recombinant WNT protein.

In embodiments, a WNT pathway activator may be termed a WNT agonist or a WNT activator. In embodiments, the WNT pathway-activator is a small molecule compound. In other embodiments a WNT pathway-activator compound or cocktail may be added to the cell culture media. In embodiments, the GSK3-inhibitor compound is a small molecule compound. In other embodiments a GSK3-inhibitor compound or cocktail may be added to the cell culture media.

In embodiments, after a minimum of 3 days of total culture duration, preferably between day 3 and 6 in culture,
the generation of brachial neurons is supported by adding a BMP pathway inhibitor, a TGFβ pathway inhibitor and retinoic acid (RA), or
the generation of lumbar neurons is supported by adding a BMP pathway inhibitor and GDF11.

In embodiments of the in-vitro method according to the invention after a minimum of 3 days of total culture duration, preferably between day 3 and 6 in culture,
the generation of brachial neurons, and optionally cervical and/or thoracic neurons (cervical and/or brachial and/or thoracic neurons), is supported by adding a BMP pathway inhibitor, a TGFβ pathway inhibitor and retinoic acid (RA), or
the generation of lumbar neurons is supported by adding a BMP pathway inhibitor and GDF11.

In embodiments, after 20 days of total culture duration, at least a fraction of the neural progenitor cells are brachial neurons, and optionally cervical and/or thoracic neurons expressing one or more of HOXC6, HOXC5, HOXC8, ISL1, CHAT and TUBB3.

In embodiments, at least a fraction of the neural progenitor cells are brachial neurons, and optionally cervical and/or thoracic neurons expressing one or more of ISL1, CHAT and TUBB3, wherein brachial and cervical neurons express HOXC5, HOXC6 and/or HOXC8, and thoracic neurons express HOXC9.

In embodiments, after 20 days of total culture duration, at least a fraction of the neural progenitor cells are brachial neurons expressing one or more of HOXC6, HOXC5, HOXC8, ISL1, CHAT and TUBB3.

In embodiments, after 20 days of total culture duration, at least a fraction of the neural progenitor cells are lumbar neurons expressing one or more of HOXC10, HOXC9, ISL1, CHAT and TUBB3.

In embodiments for the generation/induction of lumbar instead of brachial (cervical and/or brachial and/or thoracic) neurons, GDF11 is added between day 3 and day 6 (D3-D6) instead of a TGFβ pathway inhibitor and retinoic acid (RA).

Therefore, in embodiments, between day 3-6 the cell culture medium for the generation/induction of lumbar neurons comprises (i) a WNT pathway-activator (e.g., such as WNT, a WNT-activator or a GSK3 inhibitor), (ii) an FGF, (iii) and inhibitor of a BMP receptor, (iv) SHH smoothened agonist and (v) GDF11.

On the contrary, in embodiments for the generation/induction of brachial (or brachial neurons, and optionally cervical and/or thoracic neurons) instead of lumbar neurons, a TGFβ pathway inhibitor and retinoic acid (RA) are added between day 3 and day 6 (D3-D6) instead of GDF11.

Therefore, in embodiments, between day 3-6 the cell culture medium for the generation/induction of brachial (or cervical and/or brachial and/or thoracic) neurons comprises (i) a WNT pathway-activator (e.g., such as WNT, a WNT-activator or a GSK3 inhibitor), (ii) an FGF, (iii) and inhibitor of a BMP receptor, (iv) SHH smoothened agonist, (v) a TGFβ pathway inhibitor and (vi) retinoic acid (RA).

In embodiments, one or more of the first to seventh culture medium is a neurobasal (NB) medium, wherein the NB medium is preferably an about 1:1 mixture of Advanced Dulbecco's 392 Modified Eagle Medium F12 supplemented with 1×N2, and Neurobasal medium 393 supplemented with 1×B27, 2 mM L-glutamine, 75 μg/ml BSA 394 fraction V, and 0.1 mM 2-mercaptoethanol.

In embodiments, the cell culture is passaged, preferably as a single cell solution, and transferred into a Matrigel or Geltrex coated culture dish after 4-8 days in culture, preferably on day 6 of total culture, and/or after day 20-50, preferably on day 25-28 of total culture.

In embodiments, at day 25 or at day 25-28, the cell culture becomes relatively dense over earlier culture densities (almost or approximately confluent) and may be replated and/or split into new culture vessel(s)/dish(es).

In embodiments, the inhibitor of a BMP receptor is an inhibitor of a BMP type I receptor, preferably LDN193189 (LDN) and/or the inhibitor of transforming growth factor-β receptor (TGF-βR) is SB431542 (SB).

In specific embodiments, the inhibitor of a BMP receptor is LDN193189 (LDN). In specific embodiments the inhibitor of transforming growth factor-β receptor (TGF-βR) is SB431542 (SB).

In some embodiments, the inhibitor of a BMP receptor and the inhibitor of transforming growth factor-receptor are added to the media as double SMAD inhibitor cocktail (2SMADi).

In embodiments, after 6 days of total culture duration at least a fraction of the neural progenitor cells expresses SOX2 and at least a fraction of the mesodermal progenitor cells expresses PAX3, and wherein the cells segregate into neural and mesodermal progenitor clusters.

Preferably the exposure of the cell culture to the conditions and components of the second culture media (comprising one or more of a WNT pathway-activator (e.g., such as WNT/WNT-activator/GSK3-inhibitor), an FGF, SHH-smoothened agonist (SAG), a BMP pathway inhibitor, and either (i) a TGFβ pathway inhibitor and retinoic acid (RA), or (ii) GDF11) occurs for the first time after at least three days of total cell culture duration, e.g., after three days of initial culture, in the first culture media (preferably comprising a WNT pathway-activator (e.g., such as WNT/WNT-activator/GSK3-inhibitor) and an FGF). The inventors surprisingly found, that when said exposure to the factors/supplements of the second media occurs at earliest on day three, it enables first the establishment of the neuromesodermal progenitor (NMP) state in the cell culture (e.g., guided by the components of the first culture media, such as a WNT pathway-activator (e.g., such as WNT/WNT-activator/GSK3-inhibitor) and an FGF), such that the supplements of the second media can subsequently induce the desired simultaneous differentiation of the cells to both neural and Pre-somitic mesoderm (PSM) lineages.

In embodiments, after at least 6 days of total culture duration and after the consecutive exposure to the first and second culture media, at least a fraction of the cells of the present cell culture are neural progenitor cells expressing SOX2, and mesodermal progenitor cells expressing PAX3, wherein at least a fraction of the cells preferably segregate into neural and mesodermal progenitor clusters.

In embodiments, after at least 6 days of total culture duration and after the consecutive exposure to the first and second culture media, at least a fraction, or at least 10-70% of the cells of the present cell culture are neural progenitor cells expressing SOX2, and mesodermal progenitor cells expressing PAX3, wherein at least a fraction, or at least 10-70% of the cells preferably segregate into neural and mesodermal progenitor clusters.

In embodiments, at least a fraction of the neural progenitor cells expresses one or more of the factors of TUBB3, ISL1 and CHAT, and the muscle progenitor cells express one or more of the factors of DESMIN, MYOD and MYF5, after 20 days of total culture duration.

Desmin (DESMIN) is a muscle-specific class III intermediate filament protein. Homopolymers of this protein form a filamentous network connecting myofibrils to the plasma membrane and to each other.

In embodiments, at least a fraction of the neurons express TUBB3 and at least a fraction of the muscle cells express MSKF, DESMIN and/or TITIN, after 50 days of total culture duration.

Titin (TITIN) is a protein connecting the z-disk and m-lines of muscle sarcomeres.

In embodiments, from culture day 50 and after, the cell culture preferably comprises neuro-muscular junctions, wherein bundles of muscle fibers are surrounded by innervating motor neurons forming neuromuscular junctions and are preferably also (partially) interwoven with axonal structures of neural cells.

In embodiments, on and after day 50 of culture, the cell culture further comprises terminal Schwann cells and/or other spinal cord neurons, such as interneurons and/or glia cells expressing GFAP.

Neuromuscular diseases embrace a wide range of pathologies such as (i) Motor-Neuron Diseases (MNDs) like amyotrophic lateral sclerosis (ALS) and spinal muscular atrophy (SMA), (ii) specific myopathies such as Becker's and Duchenne's muscular dystrophies and (iii) auto-immune neuromuscular diseases such as myasthenia gravis. Neuromuscular diseases are caused by functional defects of the nervous system or skeletal muscle or arise by defects of the neuromuscular junction (NMJ).

In embodiments, the first cell culture comprises pluripotent stem cells of a patient, preferably iPS cells of a patient, and wherein the patient has been diagnosed with and/or is suffering from a myopathy, motor-neuron disease, an auto-immune neuromuscular disease, or another disease affecting the muscular or neuromuscular system of said patient.

The present method and model may in embodiments therefore be applied using ‘diseased’ cells derived from patients that has been diagnosed with and/or is suffering from a myopathy, motor-neuron disease, an auto-immune neuromuscular disease, or other disease affecting the muscular or neuromuscular system of a patient, such as such as spinal bulbar muscular atrophy, amyotrophic lateral sclerosis, spinal muscular atrophy, Becker's muscular dystrophy, Duchenne's muscular dystrophy, myasthenia gravis, congenital muscular dystrophy, distal muscular dystrophy, Emery-Dreifuss muscular dystrophy, Limb-girdle muscular dystrophy, Charcot-Marie-Tooth disease, a mitochondrial myopathy, a congenital or a distal myopathy, a congenital myasthenic syndrome, giant axonal neuropathy, or Lambert-Eaton myasthenic syndrome.

In specific embodiments myopathies and motor-neuron diseases comprise or is/are amyotrophic lateral sclerosis, spinal bulbar muscular atrophy and/or spinal muscular atrophy. In specific embodiments myopathy comprises or is Becker's muscular dystrophy, Duchenne's muscular dystrophy, congenital muscular dystrophy, distal muscular dystrophy, Limb-girdle muscular dystrophy, Emery-Dreifuss muscular dystrophy, Charcot-Marie-Tooth disease, a mitochondrial myopathy, a congenital and/or a distal myopathy. In specific embodiments an auto-immune neuromuscular disease is or comprises myasthenia gravis. In specific embodiments diseases affecting the muscular or neuromuscular system of a patient are or comprises giant axonal neuropathy, a congenital myasthenic syndrome and/or Lambert-Eaton myasthenic syndrome.

In embodiments, cells may be employed that are not necessarily derived from a patient, but are discovered to or are engineered to exhibit one or more genetic features that cause and/or are associated with neuromuscular conditions.

In one embodiment, the activator of a growth factor signaling pathway is a fibroblast growth factor such as basic fibroblast growth factor 2 (FGF2) or another FGF isoform like FGF8, FGF17, FGF18 etc. These fibroblast growth factors activate fibroblast growth factor signaling pathways. In an embodiment, the fibroblast growth factor is used in a concentration within in a range from about 1 to 1000 ng/ml, 10 to 500 ng/ml, 30 to 500 ng/ml, 30 to 200 ng/ml, in particular 30 to 100 ng/ml, in particular 10 to 50 ng/ml, or 10, 15, 20, 25, 30, 35, 40, 45, 50, 55, 60, 65, 70, 75, 80, 85, 90, 95, 100 ng/ml, in some embodiments between 30-50 ng/ml, or 40 ng/mL.

In a specific embodiment, the method comprises the steps of:
providing Human PSCs (e.g., in culture for at least two passages and after they reached 70% confluency), dissociated into single cells,
plating/seeding Human PSCs on dishes, preferably coated with Matrigel, at a density of 75.000-125.000/cm², adding a culture media, preferably a neurobasal (NB) medium, supplemented with a GSK-3β inhibitor and/or Wnt activator, e.g., CHIR99021 (e.g., 3 μM), and bFGF (e.g., 40 ng/ml),
changing the medium, preferably on day 3 in culture, to a medium, preferably NB medium, supplemented with a GSK-3β inhibitor and/or Wnt activator, e.g., CHIR99021 (e.g., 3 μM), and bFGF (e.g., 40 ng/ml), a TGFβ pathway inhibitor, e.g., SB431542 (SB; e.g., 10 μM), an inhibitor of BMP (e.g., 0.2 nM), e.g., LDN193189 (LDN), retinoic acid (RA; e.g., 500 nM) and SHH smoothened agonist (SAG; e.g., 500 nM) and change the medium daily, preferably until day 6 in culture,
optionally analyzing the cells by immunofluorescence for the co-expression of the NMP markers T/BRA and SOX2,
obtaining a single cell suspension from the culture, preferably on day 6 in culture, preferably by incubating the cells with Accutase, e.g., for 3 minutes at 37° C.,
seeding the cells, preferably at a density of 100.000 cells/cm² and preferably onto plates coated with Matrigel, adding a culture media, preferably a neurobasal (NB) medium, supplemented with a Rho-associated protein kinase ROCK inhibitor (e.g., 10 μM), IGF1 and HGF (e.g., both at 2 ng/ml), retinoic acid (RA; e.g., 500 nM) and SHH smoothened agonist (SAG; e.g., 500 nM),
removing the ROCK after an incubation of between 12-36 hrs. by replacing the medium with a cell culture medium, preferably NB medium, supplemented with IGF1 and HGF (e.g., both at 2 ng/ml), retinoic acid (RA; e.g., 500 nM) and SHH smoothened agonist (SAG; e.g., 500 nM), and cultivating the cells for 2-5 days, preferably until day 10 of total culture duration, and preferably changing the medium daily,
replacing the medium, preferably on day 10 in culture, with a medium, preferably NB medium, supplemented a with a gamma-secretase inhibitor (e.g., DAPT; e.g., 10 μM), with IGF1 and HGF (e.g., both at 2 ng/ml), and cultivating the cells for 2-5 days, preferably until day 14 of total culture duration with preferably changing the medium daily,
replacing the medium, preferably on day 14 in culture, with a medium, preferably NB medium, supplemented a with IGF1 and HGF (e.g., both at 2 ng/ml), and cultivating the cells for 6-11 days, preferably until day 25 of total culture duration, with preferably changing the medium every other day,
dissociating the cell culture into clumps, e.g., using TrypLE Express for 3 minutes at 37° C., preferably on day 25 in culture, and replating the cells into cell culture dishes, e.g., into 12-well-plates with 18-20 mm glass coverslips, preferably coated with Matrigel, preferably at a dilution of 1:6, using a cell culture medium, preferably NB medium, supplemented with Rho-associated protein kinase ROCK inhibitor (e.g., 10 M), IGF1 and HGF (e.g., both at 2 ng/ml), and 1×ITS, and cultivating the cells for 2-5 days, preferably until day 28 of total culture duration, and preferably changing the medium daily,
replacing the medium, preferably on day 28 in culture, with a medium, preferably NB medium, supplemented with 1×ITS, and changing the medium every other day.
optionally analyzing the formation of neuromuscular junctions between day 50 and day 100 by fluorescence immunostaining.

In specific embodiments a GSK-3β inhibitor and/or Wnt activator may be used at a concentration of 3 μM, or a concentration between 1-20, 1-10, or 1-5 M or at 1, 2, 3, 4, 5, 6, 7, 8, 9, 10, 11, 12, 13, 14, 15, 16, 17, 18, 19, 20 μM. The concentration may depend on the specific compound used.

In specific embodiments a FGF, such as bFGF may be used at a concentration of 40 ng/ml, or a concentration between 5-100 ng/ml, 10-50 ng/ml or at 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 ng/ml. The concentration may depend on the specific compound used.

In specific embodiments a TGFβ pathway inhibitor, e.g., SB431542, may be used at a concentration of 10 μM, or a concentration between 1-100 μM, 5-50 μM, 5-15 μM, or 10-25 μM or at 1, 2, 3, 4, 5, 6, 7, 8, 9, 10, 11, 12, 13, 14, 15, 16, 17, 18, 19, 20, 25, 30, 35, 40, 45, 50 μM. The concentration may depend on the specific compound used.

In specific embodiments an inhibitor of BMP, e.g., LDN193189, may be used at a concentration of 0.2 nM, or a concentration between 0.1-10 nM, or between 0.1-5 nM, or at 0.1, 0.15, 0.2, 0.25, 0.3, 0.35, 0.4, 0.45, 0.5, 0.55, 0.6, 0.65, 0.7, 0.75, 0.8, 0.9, 1, 2, 3, 4, 5, 6, 7, 8, 9, 10, 11, 12, 13, 14, 15 nM. The concentration may depend on the specific compound used.

In specific embodiments retinoic acid (RA) may be used at a concentration of 500 nM, or a concentration between 100-1,000 nM, or between 100-700 nM or at 50, 100, 150, 200, 250, 300, 350, 400, 450, 500, 550, 600, 650, 700, 750, 800, 850, 900, 950, or 1000 nM.

In specific embodiments Sonic Hedgehog (SHH) smoothened agonist (SAG) may be used at a concentration of 500 nM, or a concentration between 100-1,000 nM, or between 100-700 nM or at 50, 100, 150, 200, 250, 300, 350, 400, 450, 500, 550, 600, 650, 700, 750, 800, 850, 900, 950, or 1000 nM.

In specific embodiments an gamma-secretase inhibitor, e.g., DAPT, may be used at a concentration of 10 μM, or a concentration between 1-100 μM, 5-50 μM, 5-15 μM, or 10-25 μM or at 1, 2, 3, 4, 5, 6, 7, 8, 9, 10, 11, 12, 13, 14, 15, 16, 17, 18, 19, 20, 25, 30, 35, 40, 45, 50 μM.

Any range derived from, using end or intermediate points, or within the afore-mentioned numerical concentration ranges is also comprised by the present disclosure. The afore-mentioned concentrations may also apply to the kit embodiments described in further detail below.

In embodiments, after about 5-6 days of culture at least a fraction of the cells forms mesodermal progenitors that form ball-like structures and that at least partially segregate from the neural progenitors. In some embodiments the culture needs to be replated at this stage. Hence, in preferred embodiments, around day 6 at least a fraction of the cell culture model segregates into (distinguishable) neural and mesodermal progenitors.

In embodiments, after about 10 days of culture at least a fraction of the cells forms either clusters of motor neuron progenitors (rounder appearing colonies) or clusters of pre-somitic mesodermal progenitors (flat clusters), such that the culture comprises at least partially segregated motor neuron progenitors and pre-somitic mesodermal progenitors.

In embodiments, after about 14 days of culture at least a fraction of the cells are neural or mesodermal progenitor cells that form distinct clusters and shapes.

In embodiments, after about 25 days of culture at least a fraction of the cells has developed into motor neurons and myoblasts.

In embodiments, the combination of an inhibitor of BMP and a TGFβ pathway inhibitor may be referred to as a “dual SMAD inhibitor (cocktail)” (2SMADi). In embodiments an inhibitor of BMP is an inhibitor of BMP type I receptor (BMP I).

Further aspects of the invention relate to the cell culture model as such. The cell culture model may be defined by the method of manufacture, or by its structural features, determinable using routine measurement methods.

In one aspect, the invention relates to an in vitro cell culture model comprising segregated neural and mesodermal progenitor cells produced according to the method steps a)-b) or the method steps a)-d) of the in-vitro method according to the invention.

In embodiments of the in vitro cell culture model, at least a fraction, preferably at least 1-70%, of the neural progenitor cells expresses SOX2, and at least a fraction, preferably at least 1-70%, of the mesodermal progenitor cells express PAX3, and wherein at least a fraction of the cells segregates into neural and mesodermal progenitor clusters.

In preferred embodiments, after around 6 days in cell culture, according to the present cell culture method, the in vitro cell culture model comprises at least a fraction of cells segregated into (distinguishable) neural and mesodermal progenitor cells.

In another aspect the present invention relates to an in vitro cell culture model comprising, preferably on or after day 20 in culture, segregated partially differentiated neural and myoblast cells produced according to the method steps a)-c) or the method steps a)-e) of the method according to the invention, wherein at least a fraction of the neural cells express TUBB3, and at least a fraction of myoblast cells express DESMIN and form bundles of fibers, preferably having a length of over 50 μm, that are surrounded and at least partially interwoven with axonal structures of neural cells, preferably axonal structures of a length of over 20 μm.

In preferred embodiments, after around 20 days in cell culture, according to the present cell culture method, the in vitro cell culture model comprises at least a fraction of segregated partially differentiated neural and myoblast cells.

In another aspect the present invention relates to an in vitro cell culture model of brachial motor neurons produced according to the method of the present invention, wherein at least a fraction of the motor neurons expresses one or more of HOXC6, HOXC5, HOXC8, ISL1, CHAT and TUBB3.

In another aspect the present invention relates to an in vitro cell culture model of cervical/brachial/thoracic (cervical and/or brachial and/or thoracic) motor neurons produced according to the method of the present invention, wherein at least a fraction of the motor neurons expresses one or more of HOXC6, HOXC5, HOXC8, HOXC9, ISL1, CHAT and TUBB3.

In embodiments, at least a fraction of the motor neurons expresses one or more of ISL1, CHAT and TUBB3, wherein brachial and cervical neurons express HOXC5, HOXC6 and/or HOXC8, and thoracic neurons express HOXC9.

In another aspect, the present invention relates to an in vitro cell culture model of lumbar motor neurons produced according to the method of any one of the invention, wherein at least a fraction of the motor neurons express one or more of the factors HOXC10, HOXC9, CHAT, TUBB3, and ISL1.

In certain embodiments, a cell culture model according to the invention comprises at least a fraction of both lumbar and brachial, or lumbar and one or more of cervical and/or brachial and/or thoracic motor neurons produced according to the method of the invention.

The definition of the cell culture model by the method of manufacture is not a necessarily limiting feature. Features referring to the method of manufacture, when describing the cell-culture model, may be considered optional. As described in more detail herein, the cell culture model may be defined and determined based on structural features, either independent of or in combination with the methods of the invention.

In one aspect, the invention relates to an in vitro cell culture model, comprising (i) segregated neural and mesodermal progenitor cells, (ii) segregated partially differentiated neural and myoblast cells, and/or (iii) bundles of muscle fibers surrounded by innervating motor neurons, forming neuromuscular junctions.

In another aspect, the present invention relates to an in vitro cell culture model comprising neuro-muscular junctions generated according to the method the present invention, wherein the model comprises bundles of muscle fibers surrounded by innervating motor neurons forming neuromuscular junctions, and preferably terminal Schwann cells and/or other spinal cord neurons, such as interneurons and/or glia cells expressing GFAP, and wherein at least a fraction of the neurons express TUBB3 and at least a fraction of the muscle cells expresses MSKF, DESMIN and/or TITIN.

From day 50 on, of cell culture according to the present cell culture method, and after, embodiments of the present model preferably comprise neuro-muscular junctions, wherein the model comprises bundles of muscle fibers surrounded by innervating motor neurons, and preferably terminal Schwann cells, forming neuromuscular junctions and preferably also (partially) interwoven with axonal structures of neural cells.

In preferred embodiments, after around 50 days in cell culture, according to the present cell culture method, the in vitro cell culture model comprises bundles of muscle fibers surrounded by innervating motor neurons forming neuromuscular junctions and preferably also (partially) interwoven with axonal structures of neural cells.

On and after day 50 in cell culture, embodiments of the present model further comprise terminal Schwann cells and/or other spinal cord neurons, such as interneurons and/or glia cells expressing GFAP.

In embodiments, the model comprises bundles of muscle fibers surrounded by innervating motor neurons forming functional neuromuscular junctions, and preferably terminal Schwann cells and/or other spinal cord neurons, such as interneurons and/or glia cells expressing GFAP, and wherein at least a fraction of the neurons express TUBB3, and at least a fraction of the muscle cells expresses MSKF and/or MYF5.

In embodiments of the in vitro cell culture model according to the afore-mentioned embodiment, at least a fraction of the motor neurons form axons of between 50-3,000 μm length and at least a fraction of the muscle fibers has a length of between 100-3000 μm.

In embodiments of the in vitro cell culture model according to the afore embodiment at least a fraction of the motor neurons form axons of between 50-3,000 μm length, of between 5-5,000 μm, 10-5,000 μm, 100-1,000 μm, 100-500 μm, 25-3,000 μm length or of at least 5, 10, 15, 20, 25, 30, 35, 40, 45, 50, 55, 60, 65, 70, 75, 80, 85, 90, 95, 100, 200, 300, 400, 500, 600, 700, 800, 900, 1,000, 1,500, 2,000, 2,500, 3,000, 4,000, 5,000 μm length.

In embodiments of the in vitro cell culture model according to the afore embodiment at least a fraction of the muscle fibers have a length of between 50-3,000 μm length, of between 5-5,000 μm, 10-5,000 μm, 100-1,000 μm, 100-500 μm, 25-3,000 μm length or of at least 5, 10, 15, 20, 25, 30, 35, 40, 45, 50, 55, 60, 65, 70, 75, 80, 85, 90, 95, 100, 200, 300, 400, 500, 600, 700, 800, 900, 1,000, 1,500, 2,000, 2,500, 3,000, 4,000, 5,000 μm length.

In another aspect the present invention relates to an in vitro cell culture model comprising segregated neural and mesodermal progenitor cells, wherein at least a fraction of the neural progenitor cells expresses SOX2, and at least a fraction of the mesodermal progenitor cells expresses PAX3, and wherein at least a fraction of the cells segregates into neural and mesodermal progenitor clusters.

In another aspect the present invention relates to an in vitro cell culture model comprising segregated partially differentiated neural and myoblast cells, wherein at least a fraction of the neural cells expresses TUBB3, and at least a fraction of myoblast cells expresses DESMIN and form bundles of fibers, preferably having a length of over 50 μm, that are surrounded and at least partially interwoven with axonal structures of neural cells, preferably axonal structures of a length of over 20 μm.

In another aspect the present invention relates to an in vitro cell culture model comprising neuro-muscular junctions, wherein the model comprises bundles of muscle fibers surrounded by innervating motor neurons forming functional neuromuscular junctions, and preferably terminal Schwann cells and/or other spinal cord neurons, such as interneurons and/or glia cells expressing GFAP, and wherein at least a fraction of the neurons express TUBB3, and at least a fraction of the muscle cells expresses MSKF and/or MYF5.

In embodiments of the in vitro cell culture model according to the afore embodiment at least a fraction of the motor neurons form axons of between 50-600 μm length and at least a fraction of the muscle fibers have a length of between 100-3,000 μm.

The present cell culture model has the advantage over cell culture models of the prior art, that it can be (or can be generated to be) position specific, meaning that motor neurons with a spinal identity or a groups of identities are comprised within said model, e.g., motor neurons having only lumbar or brachial identity, or having only lumbar or cervical, brachial and/or thoracic identity, are generated. The capability of only specific motor neuron identities is especially advantageous for studying diseases and/or testing medications for motor-neuron diseases that affect only certain motor neuron identities and in each patient individually, such as amyotrophic lateral sclerosis (ALS).

In another aspect the present invention relates to an in vitro cell culture model of differentiated brachial motor neurons comprising neuro-muscular junctions, wherein at least a fraction of the motor neurons expresses one or more of HOXC6, HOXC5, HOXC8, ISL1, CHAT and TUBB3.

In embodiments said in vitro cell culture model of differentiated brachial motor neurons further comprises motor neurons and skeletal muscle cells forming neuro-muscular junctions. In embodiments said cell culture model further comprises terminal Schwann cells, which preferably also take part in and/or support the neuromuscular junctions.

In another aspect the present invention relates to an in vitro cell culture model of differentiated brachial/cervical/thoracic (cervical and/or brachial and/or thoracic) motor neurons comprising neuro-muscular junctions, wherein at least a fraction of the motor neurons expresses one or more of HOXC6, HOXC5, HOXC8, HOXC9, ISL1, CHAT and TUBB3.

In embodiments at least a fraction of the motor neurons expresses one or more of ISL1, CHAT and TUBB3, wherein brachial and cervical neurons express HOXC5, HOXC6 and/or HOXC8, and thoracic neurons express HOXC9.

In embodiments said cell culture model further comprises terminal Schwann cells, which preferably also take part in and/or support the neuromuscular junctions.

In certain embodiments a cell culture model according to the invention comprises at least a fraction of both lumbar and brachial, or lumbar and one or more of cervical and/or brachial and/or thoracic motor neurons produced according to the method of the invention.

In embodiments said in vitro cell culture model of differentiated cervical and/or brachial and/or thoracic motor neurons further comprises motor neurons and skeletal muscle cells, and preferably terminal Schwann cells, forming neuro-muscular junctions.

In another aspect the present invention relates to an in vitro cell culture model of differentiated lumbar motor neurons comprising neuro-muscular junctions, wherein at least a fraction of the motor neurons expresses one or more of the factors HOXC10, HOXC9, ISL1, CHAT, ISL1 and TUBB3.

In embodiments said in vitro cell culture model of differentiated lumbar motor neurons further comprises motor neurons and skeletal muscle cells forming neuro-muscular junctions.

In embodiments of the in vitro cell culture models according to the invention the cell culture model is a two-dimensional (2D), self-organizing neuro-muscular junction cell culture model.

In embodiments of the in vitro cell culture models according to the invention the model is established from pluripotent stem cells of a patient, preferably iPS cells of a patient, and wherein the patient has been diagnosed with and/or is suffering from myopathy, motor-neuron diseases, an auto-immune neuromuscular disease, or a disease affecting the muscular or neuromuscular system of said patient.

ALS is a heterogenous neuromuscular disease, and a disease that can affect different parts of the body in each individual patient differently. In general, motor neurons differ depending on their location of the body. In ALS this diversity is also reflected, as different motor neurons (e.g., upper motor neurons or lower motor neurons) in different body parts can be affected by ALS in a manner that is often quite individual to a patient⁵⁰.

The cell culture models of the prior art are not able to reflect the diversity of motor neurons throughout the human body and therefore are only to a limited degree useful in reflecting and studying such a diverse disease as ALS in vitro. However, the present model enables advantageously e.g., the generation of lumbar, or brachial, cervical and/or thoracic, motor neurons, which provides an advanced mode of the motor neurons of the human body, which therefore can also better reflect the specificity of motor neuron affecting diseases, such as ALS. Moreover, until now it is unclear, how the diversity of ALS phenotypes is caused and how the observed pattern of motor involvement in different ALS phenotypes can be affected pharmaceutically. The present model provides a new and improved tool to analyze the underlying causes, mechanisms and their treatment for the treatment and/or prevention even of complex motor neuron diseases, such as ALS. The model can further be used for patient stratification and/or for analyzing an individual disease outcome of a patient, treatment and/or context, such as in the context of personalized medicine context. Therefore, in embodiments the present method and/or model can be used in a personalized medicine context for the diagnosis, or the development/selection of a suitable treatment for an individual patient, from whom the initial pluripotent starting cell culture of the present model, as IPS cells, is derived.

This is especially advantageously for patients having been diagnosed with diseases such as ALS, where each patient develops an individual physiological pattern of disease outcome with e.g., an individual patterning of limbs or body regions being affected by the disease. The present methods and models facilitate the generation of position-specific cell culture models which mimic, for example, an individual phenotype of an ALS disease of a specific patient, e.g., which only affect the upper or lower body parts (arms or legs), namely, e.g., only lumbar or brachial motor neurons.

In another aspect the present invention relates to the use of the in vitro cell culture model according to the invention in drug screening, toxicity screening and/or for studying a disease, such as a myopathy, motor-neuron disease, auto-immune neuromuscular disease, or another disease affecting the muscular or neuromuscular system of a patient.

In another aspect the present invention relates to a method for testing a candidate compound, the method comprising:
a. providing a cell culture model according to the invention,
b. bringing into contact a candidate compound with said cell culture model,
c. detecting a readout, characteristic and/or one or more parameter(s) of the cell culture model,
d. optionally repeating steps a-c with one or more further candidate compounds.

In embodiments the readout or parameter in step c. comprises the viability and/or proliferation of said model. A skilled person is aware of different methods for detecting the viability and/or proliferation of a cell culture. Examples of methods for detecting the viability and/or proliferation of a cell culture are BrdU-assay, EdU-assay, MTT-assay, XTT-assay, WST-1-assay, Ki67-staining, immunocytochemistry (ICC), immunohistochemistry, immunofluorescence, Hoechst-staining, Calcein-AM staining or Trypan Blue staining.

In embodiments detecting a readout or parameter in step c. comprises using one or more of the methods of microscopy, image analysis (e.g., via a plate reader, microscope or camera), current clamp recording, analysis of optogenetic activation, immunocytochemistry (ICC), immunohistochemistry, Immunofluorescence analysis, droplet-based microfluidic technology, Fluorescence Activated Cell Sorting (FACS), IHC, ELISA and/or spectrophotometry.

Optogenetic stimulation is a genetic technique that allows the activation or inhibition of the activity of specific neuron populations using light.

In embodiments the readout or parameter may be a characteristic, a shape and/or a pattern of the present model or of certain cells comprised therein. In embodiments a non-limiting list of parameters or characteristics may be, e.g., the expression of certain genes or proteins, the developmental stage, the length, appearance and/or shape of cells, the length, organization and/or orientation of axons and/or muscle fibers, the degree of muscle fiber and/or muscle cell contraction, the transfer and/or excretion of neurotransmitters, e.g., at NMJs between cells of the model or the cell composition (e.g., the presence and/or absence of different cell types) of the model.

In embodiments, screening methods and systems may employ software and hardware as commonly used in (pharmaceutical) compound screening systems. Automated setups are known to a skilled person and may be adapted accordingly. Thus, screening formats, as already established in the art, may be employed to implement the methods and systems of the present invention.

In embodiments, the assessments are preferably performed in triplicates, to discard non-specific readouts or signals.

In another aspect, the present invention relates to a method assessing toxicity of a candidate substance, comprising:
a. providing a cell culture model according to the invention,
b. bringing into contact a candidate compound with said cell culture model,
c. identifying the candidate substance as a safe candidate substance (a substance with low or no toxic effect) when said cell culture mode exhibits no cytotoxic effect, preferably no detectable effect.

Another aspect of the present invention relates to a system for identifying a candidate substance, comprising:
a. providing a cell culture model according to the invention,
b. optionally at least one substance or a substance library, or means for providing and/or utilizing such a substance or substance library,
c. means for determining (screening) an effect of said substance or substance library on the cell culture model, such as one or more compound(s) or composition(s), a device and/or a system configured (optionally for high-throughput screening) for identifying a readout, characteristic and/or one or more parameter(s) of the cell culture model.

In another aspect the present invention relates to a kit comprising a culture medium and at least one supplement selected from the group comprising a WNT pathway-activator (e.g., such as WNT or a WNT-activator or a GSK-3 inhibitor (e.g., CHIR99021)), an FGF (e.g., bFGF), HGF, IGF1, SHH smoothened agonist (SAG), retinoic acid (RA), inhibitor of a BMP receptor, such as LDN193189, a TGFβ pathway inhibitor, GDF11, and a gamma-secretase inhibitor (e.g., DAPT).

In embodiments an FGF is Fibroblast growth factor 2, also known as basic fibroblast growth factor (bFGF). In embodiments bFGF promotes the induction/generation of neuromesodermal progenitor cells (NMPs).

In another aspect the present invention relates to a kit comprising a supplement-cocktail and/or cell culture medium, comprising:
a WNT pathway-activator,
an FGF,
a BMP pathway inhibitor,
(i) a TGFβ pathway inhibitor or (ii) GDF11,
A Sonic Hedgehog (SHH) smoothened agonist (SAG) and/or retinoic acid (RA), and
HGF and/or IGF1,
optionally comprising a gamma-secretase inhibitor.

In another aspect the present invention relates to a kit comprising a culture medium and at least one supplement-cocktail comprising:
i. a WNT pathway-activator and an FGF,
ii. SHH smoothened agonist (SAG) and retinoic acid (RA),
iii. HGF and IGF1,
iv. HGF, IGF1 and a gamma-secretase inhibitor,
v. a BMP pathway inhibitor, such as LDN193189, and a TGFβ pathway inhibitor, such as SB431542, and/or
vi. a BMP pathway inhibitor, such as LDN193189, and GDF11,
or any combination thereof.

In embodiments of the kit according to the invention the kit further comprises the in vitro cell culture model according to the invention.

In another aspect the present invention relates to a cell culture plate comprising the in vitro cell culture model according to the invention.

In another aspect the present invention relates to a cell culture medium as such, defined by one or more of the embodiments disclosed herein. Such culture medium products may be offered as such, or the components thereof offered in the form of a kit of separated by combinable compounds or substances.

In another aspect the present invention relates to a supplement cocktail as such, defined by one or more of the embodiments disclosed herein. Such supplement cocktail products may be offered as such, or the components thereof offered in the form of a kit of separated by combinable compounds or substances.

In another aspect, the present invention relates to a culture medium and/or supplement-cocktail comprising one or more of a WNT pathway activator, WNT, a WNT-activator, retinoic acid (RA), an FGF, Sonic Hedgehog (SHH) smoothened agonist (SAG), HGF, IGF1, GDF11, a GSK-3 inhibitor, a gamma-secretase inhibitor, a BMP pathway inhibitor, an inhibitor of transforming growth factor-β receptor (TGF-βRi), a Rho-associated protein kinase inhibitor (ROCKi), ITS (Insulin, Transferrin, Selenium) and a glucocorticoid.

In another aspect the present invention relates to a culture medium comprising one or more of the supplements selected from WNT, a WNT pathway activator, a WNT-activator, retinoic acid (RA), an FGF, Sonic Hedgehog (SHH) smoothened agonist (SAG), HGF, IGF1, GDF11, a GSK-3 inhibitor, a gamma-secretase inhibitor, a BMP pathway inhibitor, an inhibitor of transforming growth factor-β receptor (TGF-βRi) or an inhibitor of transforming growth factor-β receptor signaling (pathway), a Rho-associated protein kinase inhibitor (ROCKi), ITS (Insulin, Transferrin, Selenium) and a glucocorticoid.

In another aspect the present invention relates to a supplement-cocktail comprising one or more of WNT, a WNT pathway activator, a WNT-activator, retinoic acid (RA), an FGF, Sonic Hedgehog (SHH) smoothened agonist (SAG), HGF, IGF1, GDF11, a GSK-3 inhibitor, a gamma-secretase inhibitor, a BMP pathway inhibitor, an inhibitor of transforming growth factor-β receptor (TGF-βRi) or an inhibitor of transforming growth factor-β receptor signaling (pathway), a Rho-associated protein kinase inhibitor (ROCKi), ITS (Insulin, Transferrin, Selenium) and a glucocorticoid.

In specific embodiments a GSK-3β (pathway) inhibitor and/or Wnt (pathway) activator may be comprised in a culture medium and/or supplement-cocktail at a concentration of 3 μM, or a concentration between 1-20, 1-10, or 1-5 μM or at 1, 2, 3, 4, 5, 6, 7, 8, 9, 10, 11, 12, 13, 14, 15, 16, 17, 18, 19, 20 μM. The concentration may depend on the specific compound used.

Non limiting examples of Wnt pathway-activators are Wnt agonist 1, HLY78, Isoxazole 9 (Isx-9), SKL2001, Methyl Vanillate, WAY-316606, WAY-262611, Indirubin-3′-oxime (IDR30, 130), Laduviglusib (CHIR-99021, CT99021), L-Quebrachitol (L-QCT), KY19382 (A3051), CGP 57380, Isoxazole 9 (Isx-9) and CP21R7 (CP21).

Non-limiting examples of GSK-3 inhibitors are SB216763, Foretinib (GSK1363089), Cabozantinib (XL184, BMS-907351), CYC116, Laduviglusib (CHIR-99021), CCT129202, AT7519, TWS119, Indirubin (NSC 105327), Palomid 529 (P529, SG 00529), AZD5438, Dolutegravir, R547, SB415286, PHA-767491, CHIR-98014, BX-795, BX912, Tideglusib (NP031112, NP-12), BI-D1870, PF-573228, TDZD-8 (NP 01139), LY2090314, GW2580 (SC203877), GSK2334470, KY02111, AZD1080, GSK343, 1-Azakenpaullone (1-Akp), BIO (GSK-3 Inhibitor IX), Bisindolylmaleimide IX, AZD2858, AR-A014418, IM-12, GSK1324726A, Bikinin, EPZ015666, BIO-acetoxime, Kenpaullone, GSK591, CP21R7, PIM447, GSK′872, Pemrametostat, 5-bromoindole, Atuveciclib, Chonglou, GSK3179106, GSK3368715, GSK369796, Alsterpaullone, MAZ51, GSK3145095, Elraglusib (9-ING-41), BRD0705, Kaempferol, GSK3117391, Linerixibat, KY19382, Indirubin-3′-oxime, (E/Z)-GSK-3B, GSK3685032, 7-bromoindirubin-3-oxime, Paeoniae, PF-04802367, RGB-286638 und WAY-119064.

Non-limiting examples of FGF are FGF1, FGF2 (basic FGF, bFGF), SUN11602, FGF3, FGF4, FGF5, FGF6, FGF7, FGF8, FGF9, FGF10, FGF11, FGF12, FGF13, FGF14, FGF16, FGF17, FGF18, FGF19, FGF20, FGF21, FGF22, and FGF23.

In specific embodiments a FGF, such as bFGF, may be comprised in a culture medium and/or supplement-cocktail at a concentration of 40 ng/ml, or a concentration between 5-100 ng/ml, 10-50 ng/ml or at 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, or 100 ng/ml. The concentration may depend on the specific compound used.

Non-limiting examples of Rho-associated protein kinase inhibitors (ROCKi) are WAY-624704, Y-27632 2HCl, Thiazovivin, GSK429286A, Fasudil (HA-1077) HCl, H-1152 dihydrochloride, Y-27632, Azaindole 1 (TC-S 7001), RKI-1447, AT13148, GSK269962A HCl, Y-39983 HCl, Belumosudil (KD025), Ripasudil (K-115) hydrochloride dihydrate, Hydroxyfasudil (HA-1100) HCl, Netarsudil (AR-13324) 2HCl, ZINC00881524 (ROCK inhibitor).

In specific embodiments Rho-associated protein kinase inhibitors (ROCKi) may be comprised in a culture medium and/or supplement-cocktail at a concentration of 10 μM, or a concentration between 0.01-200 μM, 0.1-100 μM, 5-50 μM, 5-25 μM, 5-15 μM or 10-25 μM or at 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 200 μM. The concentration may depend on the specific compound used.

Non-limiting examples of a TGFβ pathway inhibitor are SB431542, SB525334, Ginsenoside Rh4, TGFβRI-IN-3, Lycopus, AUDA, Sulfasalazine, SB505124, Galunisertib, BIBF-0775, LDN-193189, LY2109761, GW788388, LY364947, Pirfenidone, SIS3, PD, ITD-1, TA-02, DMH1, LDN-212854, ML347, RepSox, Dorsomorphin, K02288, Vactosertib, SD-208, LDN-214117, A-83-01, R-268712, Dorsomorphin, SIS3, Halofuginone, TP0427736, LY, Galunisertib (LY2157299), LY2109761, SB525334, SB505124, GW788388, LY364947.

In specific embodiments a TGFβ pathway inhibitor, e.g., SB431542, may be comprised in a culture medium and/or supplement-cocktail at a concentration of 10 μM, or a concentration between 1-100 μM, 5-50 μM, 5-25 μM, 5-15 μM or 10-25 μM or at 1, 2, 3, 4, 5, 6, 7, 8, 9, 10, 11, 12, 13, 14, 15, 16, 17, 18, 19, 20, 25, 30, 35, 40, 45, 50 μM. The concentration may depend on the specific compound used.

Non-limiting examples of an inhibitor of BMP receptors are Ginsenoside Rh4, TGFβRI-IN-3, Lycopus, AUDA, SB431542, SB525334, Sulfasalazine, SB505124, Galunisertib, BIBF-0775, LDN-193189, LY2109761, GW788388, LY364947, Pirfenidone, SIS3, PD, ITD-1, TA-02, DMH1, LDN-212854, ML347, RepSox, Dorsomorphin, K02288, Vactosertib, SD-208, LDN-214117, A-83-01, R-268712, Dorsomorphin, SIS3, Halofuginone, TP0427736, LY, Galunisertib (LY2157299), LY2109761, SB525334, SB505124, GW788388, LY364947.

In specific embodiments a BMP pathway inhibitor, e.g., LDN193189, may be comprised in a culture medium and/or supplement-cocktail at a concentration of 0.2 nM, or a concentration between 0.1-10 nM, or between 0.1-5 nM, or at 0.1, 0.15, 0.2, 0.25, 0.3, 0.35, 0.4, 0.45, 0.5, 0.55, 0.6, 0.65, 0.7, 0.75, 0.8, 0.9, 1, 2, 3, 4, 5, 6, 7, 8, 9, 10, 11, 12, 13, 14, 15 nM. The concentration may depend on the specific compound used.

Non-limiting examples of retinoic acid (RA) are retinoic acid (RA; Tretinoin; All-trans Retinoic Acid), Isotretinoin (13-cis retinoic acid), Fenretinide, AR7, Adapalene, UVI 3003, Acitretin, Tazarotene, Bexarotene, AM580, Tamibarotene, TTNPB, Etretinate and

All trans-Retinal (Retinaldehyde).

In specific embodiments retinoic acid (RA) may be comprised in a culture medium and/or supplement-cocktail at a concentration of 500 nM, or a concentration between 100-1,000 nM, or between 100-700 nM or at 50, 100, 150, 200, 250, 300, 350, 400, 450, 500, 550, 600, 650, 700, 750, 800, 850, 900, 950, or 1000 nM.

Non-limiting examples of a SHH smoothened agonist (SAG) are Smoothened Agonist (SAG), Purmorphamine and Smoothened Agonist (SAG) HCl.

In specific embodiments SHH smoothened agonist (SAG) may be comprised in a culture medium and/or supplement-cocktail at a concentration of 500 nM, or a concentration between 100-1,000 nM, or between 100-700 nM or at 50, 100, 150, 200, 250, 300, 350, 400, 450, 500, 550, 600, 650, 700, 750, 800, 850, 900, 950, or 1000 nM.

Non-limiting examples of gamma-secretase inhibitors are Nirogacestat, DAPT, Crenigacestat, Itanapraced, L-685,458, LY411575, Avagacestat, BMS 299897, Compound E, DBZ, MRK 560, and PF 3084014 hydrobromide.

In specific embodiments a gamma-secretase inhibitor, e.g., DAPT, may be comprised in a culture medium and/or supplement-cocktail at a concentration of 10 μM, or a concentration between 1-100 μM, 5-50 μM, 5-15 μM, or 10-25 μM or at 1, 2, 3, 4, 5, 6, 7, 8, 9, 10, 11, 12, 13, 14, 15, 16, 17, 18, 19, 20, 25, 30, 35, 40, 45, 50 μM.

Non-limiting examples of glucocorticoids are prednisolone, cortisol (hydrocortisone), cortisone, methylprednisolone, dexamethasone, betamethasone, triamcinolone, deflazacort, fludrocortisone acetate, deoxycorticosterone acetate, aldosterone and beclomethasone.

In specific embodiments a glucocorticoid, e.g., prednisolone, may be comprised in a culture medium and/or supplement-cocktail at a concentration of 10 μM or a concentration between 1-100 UM, 5-50 μM, 5-15 μM, or 10-25 μM or at 1, 2, 3, 4, 5, 6, 7, 8, 9, 10, 11, 12, 13, 14, 15, 16, 17, 18, 19, 20, 25, 30, 35, 40, 45, 50μ M.

In specific embodiments ITS (Insulin, Transferrin, Selenium) may be comprised in a culture medium and/or supplement-cocktail at a concentration of 1×, or a concentration between 0.5× and 5×, or 0.5, 0.75, 1, 1.5, 2, 2.5, 3, 3.5, 4, 4.5, 5, 6, 7, 8, 9, or 10×.

In one aspect, the present invention relates to a cell culture medium and/or supplement-cocktail comprising:
(i) a GSK-3 inhibitor and/or Wnt activator, such es e.g., CHIRON/CHIR99021/Chir, preferably at a concentration of 3 μM, and/or
(ii) an FGF, preferably bFGF, preferably at a concentration of 40 ng/μL, and/or
(iii) a Rock Inhibitor (RI), preferably at a concentration of 10 μM.

In embodiments, said media can be referred to as neuromesodermal progenitor cells (NMP) induction medium. In embodiments said medium and/or supplement-cocktail may be suitable for induction of neuromesodermal progenitor cell development/differentiation.

In one aspect the present invention relates to a cell culture medium and/or supplement-cocktail comprising:
(i) B-Mercaptoethanol, and/or
(ii) N2, and/or
(iii) B27, and/or
(iv) Glutamate, and/or
(v) Penicillin and/or Streptomycin, and/or
(vi) NB medium and/DMEM-F12 medium preferably in a ratio of 1:1.

In embodiments, said medium and/or supplement-cocktail can be referred to as N2B27 medium/cocktail.

In one aspect the present invention relates to a cell culture medium and/or supplement-cocktail comprising:
(i) a SHH smoothened agonist (SAG), preferably at a concentration of 500 nM, and/or
(ii) a GSK-3 inhibitor and/or Wnt activator, such es e.g., CHIRON/CHIR99021/Chir, preferably at a concentration of 3 μM, and/or
(ii) an FGF, preferably bFGF, preferably at a concentration of 40 ng/μL, and/or
(iii) a TGFβ pathway inhibitor, such as SB431542, preferably at a concentration of 10 μM, and/or
(iv) an inhibitor of BMP, e.g., LDN193189, preferably at a concentration of 0.2 nM, and/or
(v) retinoic acid (RA), preferably at a concentration of 500 nM, and/or
(vi) N2B27 medium.

In embodiments, said medium and/or supplement-cocktail can be referred to as a neural progenitor and pre-somitic mesoderm (NP+PSM) medium/cocktail. In embodiments said medium and/or supplement-cocktail may be suitable for cultivation of neural progenitor and pre-somitic mesoderm cells.

In one aspect the present invention relates to a cell culture medium and/or supplement-cocktail comprising:
(i) a SHH smoothened agonist (SAG), preferably at a concentration of 500 nM, and/or
(ii) retinoic acid (RA), preferably at a concentration of 500 nM, and/or
(iii) HGF, preferably at a concentration of 2 ng/ml, and/or
(iv) IGF, preferably at a concentration of 2 ng/ml, and/or
(v) a Rock Inhibitor (RI), preferably at a concentration of 10 μM, and/or
(vi) N2B27 medium.

In embodiments said and/or supplement-cocktail may be referred to as motor neuron progenitor (MNP) and myoblast induction medium/cocktail. In embodiments said medium and/or supplement-cocktail may be suitable for induction of motor neuron progenitor (NMP) and myoblast development/differentiation.

In one aspect the present invention relates to a cell culture medium and/or supplement-cocktail comprising:
(i) retinoic acid (RA), preferably at a concentration of 500 nM, and/or
(ii) a SHH smoothened agonist (SAG), preferably at a concentration of 500 nM, and/or
(iii) HGF, preferably at a concentration of 2 ng/ml, and/or
(iv) IGF, preferably at a concentration of 2 ng/ml, and/or
(v) N2B27 medium.

In embodiments, said medium and/or supplement-cocktail may be referred to as motor neuron progenitor (MNP) and myoblast medium/cocktail. In embodiments said medium and/or supplement-cocktail may be suitable for cultivation and/or development/differentiation of motor neuron progenitor (MNP) and myoblast cells.

In one aspect the present invention relates to a cell culture medium and/or supplement-cocktail comprising:
(i) a gamma-secretase inhibitor e.g., DAPT, preferably at a concentration of 10 μM, and/or
(ii) HGF, preferably at a concentration of 2 ng/ml, and/or
(iii) IGF, preferably at a concentration of 2 ng/ml and/or
(iv) N2B27 medium.

In embodiments, said medium and/or supplement-cocktail may be referred to as motor neuron (MN) medium/cocktail. In embodiments said medium and/or supplement-cocktail may be suitable for cultivation and/or development/differentiation of motor neurons (MN).

In one aspect the present invention relates to a cell culture medium and/or supplement-cocktail comprising:
(i) HGF, preferably at a concentration of 2 ng/ml, and/or
(ii) IGF, preferably at a concentration of 2 ng/ml and/or
(iii) N2B27 medium.

In embodiments, said medium and/or supplement-cocktail may be referred to as motor neuron (MN)+myoblast maintenance medium/cocktail. In embodiments said medium and/or supplement-cocktail may be suitable for cultivation of motor neuron (MN) and myoblast cells (maintenance).

In one aspect the present invention relates to a cell culture medium and/or supplement-cocktail comprising:
(i) HGF, preferably at a concentration of 2 ng/ml, and/or
(ii) IGF, preferably at a concentration of 2 ng/ml and/or
(iii) a Rock Inhibitor (RI), preferably at a concentration of 10 μM, and/or
(iv) Insulin, Transferrin, Selenium (ITS), preferably 1×, and/or
(v) N2B27 medium.

In embodiments, said medium and/or supplement-cocktail may be referred to as MN+skM (motor neuron+skeletal muscle) maintenance medium/cocktail. In embodiments said medium and/or supplement-cocktail may be suitable for cultivation of motor neuron+skeletal muscle cells (maintenance).

Any range derived from, using end or intermediate points, or within the afore-mentioned numerical concentration ranges is also comprised by the present disclosure. The afore-mentioned concentrations may also apply to the kit embodiments described in further detail below.

In the context of the present disclosure the term ‘at least a fraction’ may refer to at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, to between 1-100%, 1-10%, 1-20%, 1-30%, 1-40%, 1-50%, 1-60%, 1-70%, 1-80%, 1-90% or to about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99% or 100% of a certain amount, number or population, e.g., a cell population.

The instant disclosure also includes kits, packages and multi-container units containing the herein described media and/or factors separately or as compositions.

Embodiments and features of the invention described with respect to the method, the cell culture models and kits are considered to be disclosed with respect to each and every other aspect of the disclosure, such that features characterizing the methods, may be employed to characterize the cell culture models, or kit and vice-versa. The various aspects of the invention are unified by, benefit from, are based on and/or are linked by the common and surprising finding of the development of a spatially and self-organized two-dimensional cell culture model capable of forming spinal cord neurons, skeletal muscle and preferably terminal Schwann cells, forming functional NMJs (e.g., if developed from cells of a healthy donor).

### BRIEF DESCRIPTION OF THE DRAWINGS

FIG. 1: Generation of self-organizing neural and mesodermal progenitors from human PSCs in adherent culture (Example 1).

Schematic representation of the strategy used to generate the soNMJ model from hPSCs. qPCR analysis on day 6 revealed that administration of 2SMADi from day 0-6 resulted in the differentiation of hPSCs to NPs expressing high levels of SOX2, SOX1, NKX1.2 and PAX6. Administration of 2SMADi from day 3-6 was sufficient to generate both NP cells and PSM cells expressing MEOX1, MYF5, FOXC1, FOXC2. (XM001: N=3, n=9; H1: N=1, n=3; H9: N=1, n=3). (c) Immunofluorescence analysis at day 6 of differentiation showed that exposure to 2SMADi since day 0 resulted in the generation of SOX2+ NPs in the absence of mesodermal cells. Co-expression of PAX3 and SOX2 corresponded to dorsal NPs. Conversely, exposure to 2SMADi after day 3 resulted in the segregation of NMP cells to SOX2+ NPs and PAX3+ PSM cells. (d) Immunofluorescence analysis at day 20 of differentiation showed that early exposure to 2SMADi from d0-d6 resulted in the generation of TUBB3+ neurons. Exposure to 2SMADi after day 3 resulted in the segregation of NMPs into DESMIN+ myoblast cells and TUBB3+ neurons. NPs: Neural progenitors; PSM: Pre-somitic mesoderm; 2SMADi: dual-SMAD-inhibition; soNMJ model: self-organizing neuromuscular junction model.

FIG. 2: Generation of position-specific brachial MNs corresponding to the MMC and LMC identity (Example 1).
(a) Immunofluorescence analysis at day 20 in the soNMJ model revealed the presence of MYF5+ skeletal muscle progenitors and SOX1+ NPs. The presence of MNs was shown by the expression of ISL1 and ChAT. (b) Quantification of SOX1+ NPs (20.9%±7.6%), MNs expressing ISL1 (18.9%±10.1%), and skeletal muscle progenitors expressing MYF5 (58.4%±10.7%). H1: N=2-3, n=6-15; XM001: N=2-3, n=6-16. (c) Analysis of the MN population at day 20 revealed the presence of brachial MNs co-expressing HOXC6 and ISL1. Both LMC MNs expressing SL1/FOXP1 and MMC MNs expressing ISL1/LHX3 were present in the soNMJ model. (d) Schematic illustration of MN columnar organization at the brachial spinal cord. (e) Quantification of HOXC6+/ISL1+ cells showing a prevalence of brachial MNs in the soNMJ model (80.2%±11.9%). Both MMC MNs (72.4%±14.4%), characterized by ISL1/LHX3 co-expression, and LMC MNs (25.3%±10%), characterized by ISL/FOXP1 co-expression were present. H1: N=3, n=10-11; XM001: N=1-3, n=3-9; H9: N=1, n=3-4. (f) Quantification of ISL1+ cells showing a prevalence of brachial MNs in the soNMJ model co-expressing HOXC5+/HOXC8+. In this prevalence of brachial MN condition HOXC10+ cells are absent, as expected. (g) Immunofluorescence analysis of brachial MNs expressing TUBB3, HOXC6, and ChAT at day 50. (h) Schematic illustration of the neuromuscular connectivity in the human body at the forelimb level. LMC: Lateral motor column; MMC: Median motor column.

FIG. 3: Formation of functional NMJs in the soNMJ model (Example 1).
(a) Schematic illustration of the strategy used to generate the soNMJ model from hPSCs under adherent culture conditions. (b) High-content imaging of whole well at day 50 soNMJ model The representative image depicts the self-organization of the neurons TUBB3+ (cyan) and skeletal muscle fibers that express Fast MHC (magenta) in 20 fields acquired in the same well. (c) Representative immunofluorescence image at day 50 and day 100 of differentiation showing the presence of TUBB3+ neurites in contact with a-bungarotoxin+ (αBTX+) AChR clusters on fast-twitch skeletal muscle fibers (Fast MHC+) suggesting the presence of neuromuscular junctions. (d) Quantification of the number of αBTX clusters at day 50 and day 100 of hPSC differentiation normalized to the number of Fast MHC myofibers. The number of NMJs increased significantly from day 50 (H1: 0.419±0.048; H9: 0.435±0.067; XM001: 0.447±0.051) to day 100 (H1: 0.832±0.09; H9: 0.872±0.099; XM001: 0.861±0.059). H1: N=3, n=9; H9: N=3, n=9; XM001: N=3, n=9. (e) At day 75 of differentiation, there was a spontaneous contraction of the skeletal muscles. Contraction analysis by live imaging showed that muscle contraction was increased by administering 10 jtM acetylcholine and blocked by adding the acetylcholine receptor inhibitor, curare (10 jtM). H1: N=3, n=9; XM001: N=9, n=27. soNMJ: self-organizing neuromuscular junction.

FIG. 4: Functional characterization of the soNMJ model (Example 1).
(a) The neuromuscular cultures were incubated with Fluo8-AM at day 75 for visualizing calcium transients with a spinning disk confocal microscope. Representative frames are shown for both spontaneous and 10 μM curare conditions. Individual neurons and muscle cells were identified by their morphology, and their calcium transients were plotted separately. Administration of 10 M curare blocked the calcium activity in the skeletal muscle fibers but not in the neurons, supporting that the muscle contraction was driven by the neurons through the NMJs. (b) Current clamp recording from a neuron transduced with an AAV encoding hSYN: NLS-GFP to label neuronal nuclei Static current injection caused spontaneous action potential firing, while 10 Hz stimulation with brief square pulses revealed the potential for repetitive firing of the neurons. Inset shows the first action potential of the train. (c) Current clamp recording from a neuron transduced with an AAV encoding hSYN: ChR2 (H134R)-GFP. The neuron reliably fired action potentials in response to 0.5 ms blue light (470 nm) flashes delivered at 2 Hz. Inset shows the first light-evoked action potential. (d) schematic representation of the optogenetic experiments. Neurons transduced with an AAV encoding hSYN: ChR2 (H134R)-GFP were stimulated with blue light to drive the contraction of the skeletal muscle cells. (e) Immuno-fluorescence image of day 75 soNMJ culture model showing neurons expressing ChR2-GFP 3 weeks post-transduction with an AAV encoding hSYN: ChR2 (H134R)-GFP (f) Optogenetic analysis of the soNMJ model at day 75. Every stimulation of ChR2-GFP+ neurons by 470 nm light pulse resulted in the concomitant contraction of the skeletal muscle cells. Exposure to 10 μM curare blocked the neural transmission at the NMJs resulting in the inhibition of muscle contraction even upon optogenetic stimulation. The muscle contraction response to blue light stimulation of the neurons was restored by washing out curare.

FIG. 5: Generation of an all human SMA soNMJ model (Example 1).
(a) Schematic illustration of the soNMJ differentiation from SMA type I hiPSCs. (b) Representative images of aBTX clusters in SMA iPSC derived soNMJs and control at day 50 of differentiation. (c) Quantifications at day 50 of the alphaBTX+ clusters revealed a significant reduction in the number and size of alphaBTX clusters in the SMA type I soNMJ cultures compared to H1, H9, and XM001 control lines. H1: N=3, n=9; H9: N=3, n=9; XM001: N=3, n=9; SMA pt. 1: N=3, n=9; SMA pt.2: N=3, n=9. (d) Immunofluorescence analysis using Myosin skeletal fast (Fast MHC) marker at day 50 showed compromised skeletal muscle fiber organization in the two SMA type I hiPSC lines compared to H1 hPSC control. Results are shown as the angle of deviation of each individual fiber from the average fiber orientation. H1: N=3, n=9; SMApt1: N=3, n=9; SMApt2 N=3, n=9. (e) Skeletal muscle contraction was impaired in SMA type I compared to XM001 control hiPSC line at day 50 of differentiation. H1: N=3, n=9; XM001: N=3, n=9; SMA pt1: N=3, n=9. (f) Muscle contraction analyzed in SMA type I at day 50 did not respond to 10 μM acetylcholine and 10 μM curare administration compared to H1 hPSC control line. hiPSCs: Human induced pluripotent stem cells; soNMJ: self-organizing neuromuscular junction.

FIG. 6: Scheme of one embodiment of the present method for generation of a self-organizing neural and mesodermal progenitors from human PSCs in adherent culture (Example 2).

Schematic representation of one embodiment of the strategy used to generate a soNMJ model from hPSCs. On top of the timeline the marker genes/proteins are shown that were shown to be expressed on respective days (day 0 (start; D0), D3, D6, D10, D14, D25 and D50). Below the timeline the cell culture supplements are enlisted that were added to the cell culture media on respective time points. In this embodiment the current cell culture media (NB media) was replaced on indicated timepoints, e.g., on day 3 (D3), D6, D10, D14, D25 and D50, with new media that was supplemented with the indicated factors. On the first day of culture the NB media was supplemented.

FIG. 7: The method of the invention facilitates the generation of position specific self-organizing neuromuscular junction models, comprising myofibers and either brachial or lumbar motor neurons forming neuromuscular junctions.

The method of the invention achieves this by applying a specific time schedule of administering specific factors and other media supplements to the cell culture according to the invention.

FIG. 8: Generation of neural cells with lumbar identity.

Motor neurons express from day 20 on HOXC10, ISL1, CHAT and/or TUBB3, muscle progenitors express DESMIN and/or MSKF. Acetylcholine receptor (AChR) clusters are detected by staining for a-bungarotoxin (aBTX), which when contacted by TUJ1+ neurites, suggest the formation of NMJs.

FIG. 9: Generation of functional neuromuscular junctions (NMJs) corresponding to the brachial level (day 100).

Brachial MNs express TUBB3 and/or ISL1 and HOXC6 and muscle progenitors express DESMIN and/or MSKF from day 20 on. Acetylcholine receptor (AChR) clusters are detected by staining for a-bungarotoxin (αBTX), which when contacted by TUBB3+ neurites, suggest the formation of NMJs.

### DETAILED DESCRIPTION

All cited documents of the patent and non-patent literature are hereby incorporated by reference in their entirety.

The invention relates to an in-vitro method of generating a neuro-muscular junction cell culture model, an in vitro cell culture model comprising either segregated neural and mesodermal progenitor cells, or segregated partially differentiated neural and myoblast cells, or differentiated brachial, cervical, thoracic and/or lumbar motor neurons forming neuro-muscular junctions, and the use of said models and methods in high throughput screenings and/or for studying disease.

The term “subject” or “patient” includes any mammalian subject or patient, preferably a human patient. In embodiments the subject or patient is a human or mammal diagnosed with and/or suffering from a certain disease.

The term “pluripotent stem cells” in this context covers any cells with the property of pluripotency, as understood by a skilled person. The term includes pluripotent stem cells derived from an embryo and induced pluripotent stem cells derived from other cell types such as a somatic cell (such as a skin cell, fibroblast, blood cell, or any other somatic cell). Pluripotent stem cells can generally be derived from a stem cell culture that has been established without destroying the donor embryo. Pluripotent stem cells may be healthy pluripotent stem cells derived from a healthy organism or diseased pluripotent stem cells derived from an organism suffering from a disease.

In embodiments, the initial pluripotent stem cell according to the present invention is a pluripotent or multipotent mammalian cell. Preferably, said initial pluripotent stem cell is a human induced pluripotent stem cell (iPSC). iPSCs are pluripotent stem cell that are generated directly from adult cells. iPSCs can give rise to any other cell type in the body or in the respective mammalian organism and have the capability of indefinite proliferation in cell culture. Advantageously iPSCs can be derived directly from adult cells or tissue and do not require the use of cells derived from embryos. iPSCs have the advantage that they can be generated from patient cells, such that they are patient-specific. In addition, iPSCs and iPSC-derived cells can be used for personalized drug discovery, toxicity screenings and disease research to understand the patient-specific basis of disease. In embodiments this also applies to the cell culture models of the present invention, which can be derived from human patient-specific iPSC. iPSCs are generally derived by introducing products of specific sets of pluripotency-associated genes, or “reprogramming factors”, into a particular cell type, such as the original reprogramming transcription factors Oct4 (Pou5f1), Sox2, c-Myc and Klf4. In embodiments in this common combination each of the factors can be functionally replaced by related transcription factors, or even small molecules, miRNAs, or other genes such as lineage specifiers.

Cell culture (also termed or tissue culture) is a process by which cells are grown under controlled conditions, generally outside of their natural environment. Generally, cells are cultivated in suitable vessel, e.g., plates wells or flasks, with a substrate or cell culture medium that is supplied with essential nutrients (such e.g., one or more of amino acids, carbohydrates, vitamins, minerals), and optionally with one or more of growth factors, hormones, and gases (CO₂, O₂), and regulates the physio-chemical environment (pH buffer, osmotic pressure etc.). Depending on the aim of the cell cultivation the medium can be chosen appropriately, and the skilled person is aware of suitable choices of culture conditions, such as vessel, medium, temperature and incubator etc.

NB media is commonly a 1:1 medium of Advanced Dulbecco's Modified Eagle Medium F12 (DMEM/F12, which comprises DMEM with Nutrient Mixture F-12, e.g., GIBCO) supplemented with 1×N2 supplement (e.g., GIBCO), and Neurobasal medium (e.g., GIBCO) supplemented with 1×B27 supplement (e.g., GIBCO), 2 mM L-glutamine (e.g., GIBCO), 75 μg/ml BSA fraction V, 0.1 mM 2-mercaptoethanol (e.g., GIBCO) and optional antibiotics. Neurobasal medium is a basal medium formulated to meet the specific cell culture requirements of neural cells from the postnatal and adult brain when used with GIBCORB-27® Supplement. Neurobasal Medium generally enables long- and short-term maintenance of homogeneous neuronal cell populations without the need for an astrocyte feeder layer.

Members of the fibroblast growth factor (FGF) family are involved in diverse mitogenic and cell survival functions and in a variety of biological processes, such as embryonic development, cell growth, morphogenesis and tissue repair. In embodiments an FGF is Fibroblast growth factor 2, also known as basic fibroblast growth factor (bFGF). FGF2/bFGF has been implicated in diverse biological processes, such as limb and nervous system development or wound healing.

HGF herein refers to Hepatocyte Growth Factor, which is considered to regulate cell growth, cell motility and morphogenesis in numerous cell and tissue types.

IGF1 herein refers to Insulin Like Growth Factor 1, which is considered to be similar to insulin in function and structure and is a member of a family of proteins involved in mediating growth and development.

CHIR99021 (CHIR) is a potent and highly selective inhibitor of glycogen synthase kinase 3 (GSK-3) (IC50 values are 6.7 and 10 nM for GSK-3β and GSK-3a respectively). CHIR99021 acts as Wnt activator.

An inhibitor of Glycogen Synthase Kinase 3 Beta (GSK-3B) preferably inhibits the enzymatic and/or kinase function of GSK-3β and/or its signal transduction function, and/or activates Wnt signaling pathway(s).

A Wnt agonist or Wnt activator controls and/or mimics the biological activity of Wnt and/or acts as a selective activator of Wnt signal transduction. The WNT gene family is a group of structurally related genes that encode secreted signaling proteins. These proteins have been implicated in diverse developmental processes, including the regulation of cell fate and patterning during embryogenesis.

Sonic Hedgehog (Shh)-Smoothened Agonist (SAG) HCl is a cell-permeable Smoothened (Smo) agonist and also induces proliferation of neuronal and glial precursors without affecting the differentiation pattern of newly produced cells. Shh-Smoothened agonist (SAG) is a small molecule compound.

LDN-193189 (DM3189; LDN) is a selective and highly potent small molecule BMP receptor signaling inhibitor, which inhibits the BMP type I receptors ALK1 (TGF-β Superfamily Receptor Type I), ALK2 (TGF-β Superfamily Receptor Type I), ALK3 (BMP Type-1A Receptor) and ALK6 (BMP Type-1B Receptor) with IC50s of 0.8 nM, 0.8 nM, 5.3 nM and 16.7 nM in the kinase assay, respectively. LDN-193189 inhibits the transcriptional activity of the BMP type I receptors ALK2 and ALK3. LDN-193189 is considered an inhibitor of TGF-beta/Smad signaling (TGF-beta/Smad Inhibitor).

Bone morphogenetic proteins (BMP) are multifunctional cytokines that play critical roles in maintaining tissue homeostasis. Bone morphogenetic protein (BMP) receptors are serine-threonine kinase receptors that signal through intracellular canonical and non-canonical SMAD pathways. Transforming growth factor beta (TGFbeta) family proteins also bind to BMP receptors. SB431542 (SB) is a potent and selective inhibitor of ALK5 (Transforming Growth Factor Beta Receptor 1) and is therefore a TGFβ pathway inhibitor, with IC50 of 94 nM. SB431542 is considered an inhibitor of TGF-beta/Smad signaling (TGF-beta/Smad Inhibitor).

In the context of the present invention, any TGFbeta/SMAD-signaling inhibitor may be used instead or alternatively to any one of the two herein exemplarily named compounds SB431542 and LDN-193189.

In embodiments the combination of any two inhibitors of TGF-beta/Smad signaling, e.g., such as an inhibitor of BMP receptor and a TGFβ pathway inhibitor, may be referred to as a “dual SMAD inhibitor (cocktail)” (2SMADi). In embodiments an inhibitor of BMP receptor is an inhibitor of BMP type I receptor.

SMADs (or Smads) are a family of structurally similar proteins that are the major signal transducers for transforming growth factor beta (TGFb; TGFbeta) superfamily receptors, which are critical for regulating cell development and growth.

In embodiments herein, any compound, inhibitor and/or activator compound may be a small molecule compound, such as the GSK-3beta inhibitor CHIR99021 or BMP receptor signaling inhibitor LDN-193189.

Insulin-Transferrin-Selen (ITS-G; e.g., Gibco) is commonly used as a basal medium supplement to reduce the amount of fetal calf serum (FBS) required for cell cultivation. Insulin promotes glucose and amino acid uptake, lipogenesis, intracellular transport, and protein and nucleic acid synthesis. Transferrin is an iron carrier that can also help lower toxic concentrations of oxygen radicals and hydrogen peroxide. Selenium, as sodium selenite, is a cofactor for glutathione peroxidase and other proteins and is used as an antioxidant in media.

A neuromuscular junction (or myoneural junction) is a chemical synapse between a motor neuron and a muscle fibre that allows the motor neuron to transmit a signal to the muscle fibre, causing muscle contraction. Generally, a muscle requires innervation to function-if only to maintain muscle tone and prevent atrophy. In the neuromuscular system, nerves from the central nervous system and the peripheral nervous system are interconnected and work in concert with muscles. Synaptic transmission at the neuromuscular junction begins when an action potential reaches the presynaptic terminal of a motoneuron, which activates voltage-gated calcium channels to allow calcium ions to enter the neuron. The calcium ions bind to sensor proteins on synaptic vesicles, triggering fusion of the vesicles with the cell membrane and subsequent release of neurotransmitters from the motoneuron into the synaptic cleft. In vertebrates, motoneurons release acetylcholine (ACh), a low molecular weight neurotransmitter that diffuses across the synaptic cleft and binds to nicotinic acetylcholine receptors (nAChRs) on the cell membrane of the muscle fibre (so called sarcolemma). nAChRs are ionotropic receptors, which serve as ligand-gated ion channels. Binding of ACh to the receptor can depolarize the muscle fibre and trigger a cascade that eventually leads to muscle contraction.

Any disorder that affects synaptic transmission between a motor neuron and a muscle cell is included under the umbrella term neuromuscular disorders. These disorders can be inherited or acquired and vary in severity and mortality. In general, most of these disorders are caused by mutations or autoimmune diseases. Neuromuscular junction disorders can have a genetic origin and/or of autoimmune in origin. Genetic disorders, such as congenital myasthenic syndrome, can result from mutated structural proteins that form the neuromuscular junction, while autoimmune diseases, such as myasthenia gravis, occur when antibodies to nicotinic acetylcholine receptors are formed on the sarcolemma. Neuromuscular diseases are usually humoral, B-cell mediated autoimmune diseases in which an antibody is mistakenly produced against a motor neuron or muscle fiber protein that interferes with synaptic transmission or signalling.

A moto(r) neuron is a neuron whose cell body is located in the motor cortex, brainstem, or spinal cord and whose axon (fiber) projects into the spinal cord or outside the spinal cord to directly or indirectly control effector organs, primarily muscles and glands. There are two types of motoneurons-upper motoneurons (cerebral cortex and brainstem) and lower motoneurons (anterior grey column, anterior nerve roots (spinal lower motor neurons), cranial nerve nuclei of the brainstem and cranial nerves with motor function). Axons of upper motoneurons synapse onto interneurons in the spinal cord and occasionally directly onto lower motoneurons. Axons of lower motoneurons are efferent nerve fibres that conduct signals from the spinal cord to organs. Types of lower motoneurons include alpha motor neurons, beta motor neurons, and gamma motor neurons. When motor neurons innervate muscle fibres, said muscle fibres can undergo many action potentials in the time it takes for a single muscle twitch. Innervation occurs at a neuromuscular junction.

Terminal Schwann cells are non-myelinating glial cells localized to the neuromuscular junction.

Myasthenia gravis is an autoimmune disease in which the body produces antibodies against either the acetylcholine receptor (AchR) (in 80% of cases) or the postsynaptic muscle-specific kinase (0-10% of cases).

Lambert-Eaton myasthenic syndrome (LEMS) is an autoimmune disorder affecting the presynaptic portion of the neuromuscular junction. This rare disease can be characterized by a unique triad of symptoms: proximal muscle weakness, autonomic dysfunction, and areflexia.

Congenital myasthenic syndromes (CMS) are very similar in function to both MG and LEMS, but the main difference between CMS and these diseases is that CMS is genetic.

ALS is a heterogenous disease that affects different parts of the body in each individual patient differently. In general, motor neurons differ depending on their location of the body. Hence, this diversity can also be seen in ALS, where different motor neurons in different body parts can be affected by ALS in a manner that is often individual to a patient 50. The patterning of motor involvement in different ALS phenotypes can be divided into bulbar-onset ALS, upper motor neuron and lower motor neuron involvement is observed in the bulbar muscles, in spinal-onset ALS, patchy upper motor neuron and lower motor neuron involvement is observed in all limbs, while in progressive muscular atrophy, lower motor neuron in arms and legs are involved, often proximally. In primary lateral sclerosis, upper motor neuron of arms and legs are primarily involved, but later in the disease, discrete lower motor neuron involvement can be detected. In pseudopolyneuritic ALS, only lower motor neuron restricted to the distal limbs are involved, while in hemiplegic ALS, unilateral upper motor neuron involvement with sparing of the face, and sometimes discrete lower motor neuron involvement, can be observed. In flail arm syndrome, lower motor neuron involvement is restricted to the upper limbs, but mild upper motor neuron signs can be detected in the legs. In flail leg syndrome, lower motor neuron involvement is restricted to the lower limbs, and is often asymmetric⁵⁰. Upper motor neurons in primary motor cortex and bulbospinal lower motor neurons are the preferentially affected sites in ALS. However, also neurons in the frontotemporal cortex can often be affected, also falling under the phenotype of dementia-ALS spectrum⁵⁰.

High Throughput Screening (HTS) is a method of scientific experimentation that is particularly relevant to the fields of biology and chemistry. For example, a combination of robotics and other specialized laboratory hardware may be used to provide systems that enable a high throughput screening approach. In the context of the invention, HTS enables a skilled person to effectively screen a great number of organisms or parts thereof in vivo or in vitro, simultaneously or sequentially. Commonly used labware or test vessels of HTS are 6-, 24-, 48-well plates and microtiter plates, which is a container, possibly disposable and made of plastics, having a grid of small, open wells called wells. In some embodiments, microplates are used for HTS and have either 96, 192, 384, 1536, 3456 or 6144 wells. These are all multiples of 96 and reflect the original 96-well microplate with 8×12 wells spaced 9 mm apart. In embodiments, 24- or 48-well plates are used to set up the intended high-throughput in vivo or in vitro screening. Most of the wells contain test items, according to the nature of the experiment, which may be adapted according to the nature of the test and/or reference organisms described herein, and the library of substances to be tested as candidate substances. A screening facility will typically have a library of stock plates, the contents of which are carefully catalogued and each of which may have been generated by the laboratory or purchased from a commercial source.

For example, to prepare an assay, each well of a plate may be seeded with a biological entity on which the experiment is to be performed, such as the cells, a cell culture model, animals or embryos of the test and/or reference organisms described herein. After an incubation period to allow the biological material to absorb, bind to, or otherwise react (or not) with the substances in the wells, e.g., a candidate drug or compound, measurements are made over the wells of the plate, either manually or by an automated procedure. Manual measurements may be necessary if microscopy is used to look for changes or defects in embryonic development caused by the candidate compounds; software may also be used to identify relevant effects. In some embodiments, a specialized automated analysis machine may be used. In dependence on the results of an HTS, follow-up assays may be performed, for example within the same screen, by identifying wells in which a desired reaction has occurred (also known as “hits”), transferring the reaction components to new assay plates and repeating the experiment.

Means for automating the present invention will also be known to those skilled in the art. Automation is an advantageous element in HTS. Typically, an integrated system consisting of one or more robots will transport assay microplates from station to station for sample and reagent addition, mixing, incubation, and finally readout or detection. An HTS system can usually prepare, incubate and analyze multiple plates in parallel, further speeding up the process.

The term “candidate substance” shall mean any substance used for (preferably high-throughput) screening. The compound characteristics, i.e. structure, purity and quantity, may be stored in a chemical library database for reference. A candidate substance may further refer to any substance which the skilled person finds suitable for the contemplated method and/or for treating a disorder that affects synaptic transmission between a motor neuron and a muscle cell.

The term “toxic effect” shall mean any adverse effect of the candidate substance produced in the respective organism, cell culture model or part thereof. A candidate substance preferably does not affect the survival of the cell culture model negatively. In embodiments it may be desired that a candidate substance changes a certain parameter of the cell culture model, e.g., in case of a diseased cell culture model towards a more healthy or normal parameter range. In embodiments the present method may comprise a control cell culture models established from healthy stem cells (e.g., of a healthy donor) and at least one control cell culture models established from diseased stem cells (e.g., of a diseased donor or artificially engineered).

### Further Embodiments and Aspects of the Invention

Embodiment 1: An in vitro method of generating a neuro-muscular junction cell culture model, comprising:
   d) cultivating a first cell culture comprising pluripotent stem cells (PSCs), for at least 3 days, in a first culture medium, comprising:
      a WNT pathway-activator, and
      FGF,
   e) replacing the first culture medium by a second culture medium, comprising:
      a BMP pathway inhibitor, and (i) a TGFβ pathway inhibitor or (ii) GDF11,
      Sonic Hedgehog (SHH) smoothened agonist (SAG) and/or retinoic acid (RA),
      a WNT pathway-activator, and
      FGF, HGF, and/or IGF1, and
      cultivating the cells for at least 3 days,
      thereby generating a cell culture model comprising segregated neural and mesodermal progenitor cells, and/or differentiated muscle cells with innervating motor neurons, forming neuromuscular junctions.
Embodiment 2: The in vitro method according to embodiment 1, wherein the method further comprises step c):
   replacing the at least second culture medium by a culture medium comprising:
      HGF and/or IGF1, and optionally a gamma-secretase inhibitor (e.g., DAPT), and cultivating the cells for at least 4 days, and optionally replacing the culture medium by a medium suitable for maintenance of the cell culture model.
Embodiment 3: The in vitro method according to embodiments 1 or 2, wherein the cell culture model is a two-dimensional (2D), self-organizing neuro-muscular junction cell culture model.
Embodiment 4: The in vitro method according to any one of embodiments 1-3, wherein step b) further comprises replacing after 2-5 days the second culture medium by a third culture medium comprising retinoic acid and/or SAG, and optionally HGF and/or IGF1, and cultivating the cells for 2-5 days.
Embodiment 5: The in vitro method according to any one of embodiments 1-4, wherein the method comprises the steps of:
   a) Cultivating a first cell culture comprising pluripotent stem cells (PSCs; preferably iPS cells, more preferably iPSCs of a patient), for at least 3 days, in a first culture medium, comprising:
      WNT pathway-activator, and
      FGF,
   b) replacing the first culture medium by a second culture medium, comprising one or more of the group comprising a WNT pathway-activator, an FGF, SHH smoothened agonist (SAG), a BMP pathway inhibitor, and either (i) a TGFβ pathway inhibitor and retinoic acid (RA), or (ii) GDF11, and cultivating the cells for 2-4 days, preferably for 3 days,
   c) replacing the second culture medium by a third culture medium comprising retinoic acid, SAG, HGF and IGF1, and cultivating the cells for 2-5 days, preferably for 4 days, wherein on the first day a Rho-associated protein kinase inhibitor (ROCKi) is added to the culture medium for 6-24 hours,
   d) replacing the third culture medium by a fourth culture medium comprising HGF and IGF1, and optionally a gamma-secretase inhibitor, and cultivating the cells for 1-5 days, preferably for 4 days,
   e) replacing the fourth culture medium by a fifth culture medium comprising HGF and IGF1, and cultivating the cells for at least 11 days,
   f) replacing the fifth culture medium by a sixth culture medium comprising a ROCK inhibitor, ITS (Insulin, Transferrin, Selenium), HGF and IGF1, and cultivating the cells for 1-4 days, preferably for 3 days, and
   g) replacing the sixth culture medium by a seventh culture medium supplemented with ITS and cultivating the cells for at least one day.
Embodiment 6: The in vitro method according to any one of the preceding embodiments, wherein after 3 days of total culture duration, preferably between day 3 and 6 in culture,
   the generation of brachial neurons and optionally cervical and/or thoracic neurons, is supported by adding a BMP pathway inhibitor, a TGFβ pathway inhibitor and retinoic acid (RA), or
   the generation of lumbar neurons is supported by adding a BMP pathway inhibitor and GDF11.
Embodiment 7: The in vitro method according to any one of the preceding embodiments, wherein after 6 days of total culture duration at least a fraction of the neural progenitor cells expresses SOX2 and at least a fraction of the mesodermal progenitor cells expresses PAX3, and wherein the cells segregate into neural and mesodermal progenitor clusters.
Embodiment 8: The in vitro method according to any one of the preceding embodiments, wherein after 20 days of total culture duration, at least a fraction of the neural progenitor cells are brachial neurons and optionally cervical and/or thoracic neurons, expressing one or more of HOXC6, HOXC5, HOXC8, HOXC9, ISL1, CHAT and TUBB3.
Embodiment 9: The in vitro method according to any one of embodiments 1-7, wherein after 20 days of total culture duration, at least a fraction of the neural progenitor cells are lumbar neurons expressing one or more of HOXC10, HOXC9, ISL1, CHAT and TUBB3.
Embodiment 10: The in vitro method according to any one of the preceding embodiments, wherein:
   a. the cell culture is passaged, preferably as a single cell solution, and transferred into a Matrigel or Geltrex coated culture dish after 4-8 days in culture, preferably on day 6 of total culture, and/or after day 20-50, preferably on day 25-28 of total culture, and/or
   b. one or more of the first to seventh culture medium is a neurobasal (NB) medium, wherein the NB medium is preferably an about 1:1 mixture of Advanced Dulbecco's 392 Modified Eagle Medium F12 supplemented with 1×N2, and Neurobasal medium 393 supplemented with 1×B27, 2 mM L-glutamine, 75 μg/ml BSA 394 fraction V, and 0.1 mM 2-mercaptoethanol.
Embodiment 11: The in vitro method according to any one of the preceding embodiments, wherein the inhibitor of a BMP receptor is an inhibitor of a BMP type I receptor, preferably LDN193189 (LDN), and/or wherein the inhibitor of transforming growth factor-β receptor (TGF-βR) is SB431542 (SB).
Embodiment 12: The in vitro method according to any one of the preceding embodiments, wherein:
   c. after 20 days of total culture duration at least a fraction of the neural progenitor cells expresses one or more of the factors of TUBB3, ISL1 and CHAT, and the muscle progenitor cells express one or more of the factors of DESMIN, MYOD and MYF5, and/or
   d, wherein after 50 days of total culture duration at least a fraction of the neurons express TUBB3 and at least a fraction of the muscle cells express MSKF, DESMIN, and/or TITIN.
Embodiment 13: The in vitro method according to any one of the preceding embodiments, wherein the first cell culture comprises pluripotent stem cells of a patient, preferably iPS cells of a patient, and wherein the patient has been diagnosed with and/or is suffering from a myopathy, motor-neuron disease, an auto-immune neuromuscular disease, or another disease affecting the muscular or neuromuscular system of said patient.
Embodiment 14: An in vitro cell culture model comprising segregated neural and mesodermal progenitor cells produced according to the method of any one of the preceding embodiments.
Embodiment 15: The in vitro cell culture model according to the preceding embodiment, wherein at least a fraction of the neural progenitor cells expresses SOX2, and at least a fraction of the mesodermal progenitor cells express PAX3, and wherein at least a fraction of the cells segregate into neural and mesodermal progenitor clusters.
Embodiment 16: An in vitro cell culture model comprising segregated partially differentiated neural and myoblast cells produced according to the method of embodiments 1-13, wherein at least a fraction of the neural cells express TUBB3, and at least a fraction of myoblast cells express DESMIN and form bundles of fibers, preferably having a length of over 50 μm, that are surrounded by and/or at least partially interwoven with axonal structures of neural cells, preferably axonal structures of a length of over 20 μm.
Embodiment 17: An in vitro cell culture model of brachial, and optionally cervical and/or thoracic, motor neurons produced according to the method of any one of embodiments 1-13, wherein at least a fraction of the motor neurons express one or more of HOXC6, HOXC5, HOXC8, ISL1, CHAT and TUBB3.
Embodiment 18: An in vitro cell culture model of lumbar motor neurons produced according to the method of any one of embodiments 1-13, wherein at least a fraction of the motor neurons expresses one or more of the factors HOXC10, HOXC9, CHAT, TUBB3, and ISL1.
Embodiment 19: An in vitro cell culture model comprising neuro-muscular junctions generated according to the method of any one of embodiments 1-13, wherein the model comprises bundles of muscle fibers surrounded by innervating motor neurons forming neuromuscular junctions, and preferably other spinal cord neurons, such as interneurons and/or glia cells expressing GFAP, and wherein at least a fraction of the neurons express TUBB3 and at least a fraction of the muscle cells expresses MSKF, DESMIN and/or TITIN.
Embodiment 20: The in vitro cell culture model according to the preceding embodiment, wherein at least a fraction of the motor neurons form axons of 50-3000 μm length and at least a fraction of the muscle fibers has a length of 100-3000 μm.
Embodiment 21: An in vitro cell culture model comprising segregated neural and mesodermal progenitor cells, wherein at least a fraction of the neural progenitor cells expresses SOX2, and at least a fraction of the mesodermal progenitor cells expresses PAX3, and wherein at least a fraction of the cells segregates into neural and mesodermal progenitor clusters.
Embodiment 22: An in vitro cell culture model comprising segregated partially differentiated neural and myoblast cells, wherein at least a fraction of the neural cells expresses TUBB3, and at least a fraction of myoblast cells expresses DESMIN and form bundles of fibers, preferably having a length of over 50 μm, that are surrounded by and/or at least partially interwoven with axonal structures of neural cells, preferably axonal structures of a length of over 20 μm.
Embodiment 23: An in vitro cell culture model comprising neuro-muscular junctions, wherein the model comprises bundles of muscle fibers surrounded by innervating motor neurons forming functional neuromuscular junctions, and preferably terminal Schwann cells and/or other spinal cord neurons, such as interneurons and/or glia cells expressing GFAP, and wherein at least a fraction of the neurons express TUBB3, and at least a fraction of the muscle cells expresses MSKF and/or MYF5.
Embodiment 24: The in vitro cell culture model according to the preceding embodiment, wherein at least a fraction of the motor neurons form axons of 50-600 μm length and at least a fraction of the muscle fibers has a length of 100-3000 μm.
Embodiment 25: An in vitro cell culture model of differentiated brachial, and optionally cervical and/or thoracic, motor neurons, comprising neuro-muscular junctions, wherein at least a fraction of the motor neurons expresses one or more of HOXC6, HOXC5, HOXC8, ISL1, CHAT and TUBB3.
Embodiment 26: An in vitro cell culture model of differentiated lumbar motor neurons comprising neuro-muscular junctions, wherein at least a fraction of the motor neurons expresses one or more of HOXC10, HOXC9, ISL1, CHAT, ISL1 and TUBB3.
Embodiment 27: The in vitro cell culture model according to any one of embodiments 14-26, wherein the cell culture model is a two-dimensional (2D), self-organizing neuro-muscular junction cell culture model.
Embodiment 28: The in vitro cell culture model according to any one of embodiments 14-17, wherein the model is established from pluripotent stem cells of a patient, preferably iPS cells of a patient, and wherein the patient has been diagnosed with and/or is suffering from myopathy, motor-neuron diseases, an auto-immune neuromuscular disease, or a disease affecting the muscular or neuromuscular system of said patient.
Embodiment 29: Use of the in vitro cell culture model according to any one of embodiments 14-28 in drug screening, toxicity screening and/or for studying a disease, such as a myopathy, motor-neuron disease, auto-immune neuromuscular disease, or another disease affecting the muscular or neuromuscular system of a patient.
Embodiment 30: A kit comprising a culture medium, and at least one supplement selected from the group consisting of a WNT pathway-activator (e.g., CHIR99021), an FGF (e.g., bFGF), HGF, IGF1, SHH smoothened agonist (SAG), retinoic acid (RA), inhibitor of a BMP receptor, such as LDN193189, a TGFβ pathway inhibitor, GDF11, and a gamma-secretase inhibitor (e.g., DAPT).
Embodiment 31: The kit according to the preceding embodiment comprising a culture medium and at least one supplement-cocktail comprising:
   i. a WNT pathway-activator, and an FGF,
   ii. SHH smoothened agonist (SAG) and retinoic acid (RA),
   iii. HGF and IGF1,
   iv. HGF, IGF1 and a gamma-secretase inhibitor,
   v. a BMP pathway inhibitor, such as LDN193189, and a TGFβ pathway inhibitor, such as SB431542, and/or
   vi. a BMP pathway inhibitor, such as LDN193189, and GDF11,
   or any combination thereof.
Embodiment 32: The kit according to embodiments 30-31, further comprising the in vitro cell culture model according to one or more of embodiments 14-28.
Embodiment 33: A cell culture plate comprising the in vitro cell culture model according to one or more of embodiments 14-28.

The embodiments disclosed above may also represent an aspect of the invention. Any feature disclosed in the context of any given aspect or embodiment may be combined with other embodiments or aspects of the invention.

### EXAMPLES

The invention is further described by the following examples. These are not intended to limit the scope of the invention but represent preferred embodiments of aspects of the invention provided for greater illustration of the invention described herein.

### Example 1

### Methods

### Human Pluripotent Stem Cell Lines and Culture Conditions

The female H9 and male H1 hPSC lines (obtained from WiCell and approved for use in this project by the Regulatory Authority for the Import and Use of Human Embryonic Stem Cells in the Robert Koch Institute (AZ:3.04.02/0123) and the female XM001 iPSC line 48 were cultured in mTESR1 medium (Stem Cell Technologies) on Geltrex LDEV-Free hESC-qualified reduced growth factor basement membrane matrix (Life Technologies) at 37° C. and 5% CO2. We obtained the SMA patient 1 line (SMA E1C4) 49 from Emory University and the SMA patient line 2 (CS32iSMAn3) from Cedars-Sinai. All the patient cell lines and protocols were approved by review boards at Emory University and Cedars-Sinai Medical Center. Cell lines were checked for normal karyotype and were mycoplasma free. The cells were passaged twice a week using Versene solution (Thermo Fisher).

### Method Details

In Vitro Generation of NMPs from Human Pluripotent Stem Cells

Human PSCs were grown for at least two passages and after they reached 70% confluency, they were dissociated into single cells using Accutase (Life Technologies). Single cells were counted using the Countess II automated cell counter (Thermo Fisher) and were plated on 35 mm cell culture dishes (Corning) coated with Matrigel (Life Technologies) at a density of 75.000-125.000/cm². The initial plating concentration of the PSCs was adjusted depending on the growth rate of the specific human pluripotent stem cell line (XM001: 75.000/cm2; H9 line: 110.000/cm2; H1 line: 100.000/cm2;).

For NMPs differentiation, Human PSCs were plated in neurobasal (NB) medium supplemented with 10 μM Rock inhibitor (Tocris Bioscience), 3 μM CHIR99021 (Tocris Bioscience; inhibitor of glycogen synthase kinase and Wnt activator) and 40 ng/ml bFGF (Peprotech). NB is a 1:1 medium of Advanced Dulbecco's Modified Eagle Medium F12 supplemented with 1×N2 (GIBCO), and Neurobasal medium (GIBCO) supplemented with 1×B27 (GIBCO), 2 mM L-glutamine (GIBCO), 75 μg/ml BSA fraction V (Sigma), 0.1 mM 2-mercaptoethanol (GIBCO).

The next day ROCK inhibitor was removed and the cells were maintained in NB medium supplemented with 3 μM CHIR99021 (inhibitor of glycogen synthase kinase, Wnt activator) and 40 ng/ml bFGF (Peprotech) until day 3. The medium was changed daily. On day 3 the cells were analyzed by immunofluorescence for the co-expression of the NMP markers T/BRA and SOX2. To test the effect of 2SMADi on NMPs, the cells were plated in neurobasal (NB) medium supplemented with 10 M Rock inhibitor (Tocris Bioscience), 3 μM CHIR99021 (inhibitor of glycogen synthase kinase and Wnt activator; Tocris Bioscience), and 40 ng/ml bFGF (Peprotech), 10 μM SB431542 (Biozol) and 0.2 nM LDN193189 (MedChem). The next day Rock inhibitor was removed and the cells were maintained in NB medium supplemented with 3 μM CHIR99021, 40 ng/ml bFGF (Peprotech), 10 μM SB431542 (Biozol) and 0.2 nM LDN193189 (MedChem) until day 3. The medium was changed daily.

In Vitro Generation of soNMJ Model.

At day 3 of NMPs differentiation, the medium was changed to NB supplemented with 3 μM CHIR99021 (inhibitor of glycogen synthase kinase and Wnt activator; Tocris Bioscience) and 40 ng/ml bFGF (Peprotech), 10 μM SB431542 (Biozol) and 0.2 nM LDN193189 (MedChem), 500 nM RA (Sigma) and 500 nM SAG (Calbiochem Millipore). The medium was changed daily until day 6.

At day 6, a single cell suspension was obtained by incubating the cells with Accutase (Life Technologies) for 3 minutes at 37° C. A density of 100.000 cells/cm2 was seeded into 35 mm plates (IBIDI) coated with Matrigel (Life Technologies). The cells were plated in NB medium with 10 μM Rho-associated protein kinase ROCK inhibitor (Tocris Bioscience), 2 ng/ml IGF1, 2 ng/ml HGF (Peprotech), 500 nM RA (Sigma) and 500 nM SAG (Calbiochem Millipore).

The following day ROCK inhibitor was removed and the cells were maintained in NB medium supplemented with 2 ng/ml IGF1, 2 ng/ml HGF (Peprotech), 500 nM RA (Sigma) and 500 nM SAG (Calbiochem Millipore). The medium was changed daily until day 10. At day 10, the medium was replaced with NB supplemented with 10 μM DAPT, 2 ng/ml IGF1, and 2 ng/ml HGF (Peprotech) until day 14. The medium was changed daily until day 14.

At day 14, DAPT was removed and the cells were kept in NB medium with 2 ng/ml IGF1 and 2 ng/ml HGF (Peprotech). The medium was changed every other day until day 25.

At day 25, the cells were dissociated into clumps using TrypLE Express (Life Technologies) for 3 minutes at 37° C. and replated from the 35 mm plates (IBIDI) into 12-well-plates with 18-20 mm glass coverslips coated with Matrigel (Life Technologies). The clumps were plated with a dilution of 1:6 using NB medium supplemented with 10 μM Rho-associated protein kinase ROCK inhibitor (Tocris Bioscience), 2 ng/ml IGF1, 2 ng/ml HGF (Peprotech) and 1×ITS (Life Technologies). The cells were kept in this medium for three days (until day 28) and the medium was changed daily.

After day 28 the cells were maintained with NB medium supplemented with 1×ITS (Life Technologies), and the medium was changed every other day.

The formation of neuromuscular junctions was analyzed between day 50 and day 100 by fluorescence immunostaining.

### In Vitro Generation of Brachial Motor Neurons.

At day 3 of npNMPs (neuronal primed NMPs) differentiation, the medium was changed to NB supplemented with 3 μM CHIR99021 (inhibitor of glycogen synthase kinase and Wnt activator; Tocris Bioscience) and 40 ng/ml bFGF (Peprotech), 10 μM SB431542 (Biozol) and 0.2 nM LDN193189 (MedChem), 500 nM RA (Sigma) and 500 nM SAG (Calbiochem Millipore). The medium was changed daily until day 6. At day 6, a single cell suspension was obtained by incubating the cells first with Versene (LifeTechnologies) for 3 minutes at 37° C. Afterwards, Versene was removed and the cells were incubated with Accutase (Life Technologies) for 3 minutes at 37° C. A density of 100.000 cells/cm² was seeded into 35 mm plates (Corning) coated with Matrigel (Life Technologies). The cells were plated in NB medium with 10 μM Rho-associated protein kinase 444 ROCK inhibitor (Tocris Bioscience), 500 nM RA (Sigma) and 500 nM SAG (Calbiochem Millipore). The following day ROCK inhibitor was removed and the cells were maintained in NB medium supplemented with 500 nM RA (Sigma) and 500 nM SAG (Calbiochem Millipore). The medium was changed daily until day 10. At day 10, the medium was replaced with NB supplemented with 10 μM DAPT until day 14. At day 14, DAPT was removed and the cells were kept in NB medium for a maximum of 50 days.

### Fluorescence Immunostainings Analysis

The cells were washed once with PBS and fixed with 4% PFA for 20 minutes at 4° C. Then PFA was removed and the cells were washed three times with PBS with 0.3% Triton X-100 (Sigma Aldrich) and blocked in PBS with 4% Bovine Serum Albumin (BSA) (Sigma Aldrich) and 0.3% Triton X-100 (Sigma Aldrich) for 1 hour at room temperature. Alternatively, the cells could be maintained at 4° C. in blocking solution for up to two weeks. After blocking, the cells were incubated with primary antibodies overnight at 4° C. Primary antibodies were washed three times with PBS with 0.3% Triton X-100 for 5 min and then the cells were incubated with secondary antibodies for 1-2 hours at room temperature. After the incubation with the secondary antibody, the cells were washed three times with PBS with 0.3% Triton X-100 for 5 min and incubated with DAPI in PBS. After the incubation with DAPI, an additional washing with PBS was performed before mounting the coverslips on glass slides. Fluorescent images were acquired using a Leica SP8 confocal microscope.

### High-Content Image Analysis

An Opera Phenix device (PerkinElmer) was used to perform high-content confocal imaging analysis of the neuromuscular culture. To this end, the cells were re-plated at day 25 on 96-well plates (PerkinElmer), culture until day 50 or day 100 and then fixed and stained as described above. The entire dish could be scanned and imaged with the Opera Phenix and multiple fields from the same well were acquired. Data processing and analysis was performed using the Harmony software (PerkinElmer).

### Reverse Transcription—Quantitative PCR Analysis

Total RNA was isolated from cells growing on Matrigel using the RNeasy kit (QIAGEN) according to the manufacturer's instructions and digested with DNase I (QIAGEN) to remove genomic DNA. First strand cDNA synthesis was performed with Superscript Ill system (Invitrogen) using random primers and amplified using Platinum SYBR-Green (Invitrogen). For qPCR the Applied Biosystems Quantstudio 6 Flex Real-Time PCR system was used. PCR primers were designed using NCBI Primer-Blast software, using exon-spanning junctions (Table S3). Expression values for each gene were normalized against GAPDH, using the delta CT method and standard deviations were calculated and plotted using Prism 8 software (GraphPad). Error bars represent standard deviation across three biological replicate samples.

Calcium Imaging with Fluo8

Between day 50 and day 100, the cells were incubated at 37° C. with 4 M of the cell permeant calcium indicator Fluo-8 AM (Abcam). After 30 minutes of incubation, the cells were washed two times with culture medium (NB without phenol red) and left to recover for 15 min in the incubator prior to the start of any optical recording. During acquisition, the samples were kept at 37° C. with 5% CO2. Fluorescent time series images were acquired with a CSU-W1 spinning disk confocal microscope. The calcium transients were recorded for 2-3 minutes at 20 frames per second in three different conditions: without any treatment, with addition of 10 M Acetylcholine (Sigma Aldrich) and with addition of 10 μM Curare (Sigma Aldrich).

Analysis of the calcium activity was performed by measuring the variation of fluorescence intensity in each ROI with ImageJ. ROIs were detected manually, and neurons and muscle cells could be distinguished by morphology. The variation of fluorescence intensity for each individual ROI was then plotted in Prism 8 (GraphPad).

### Electrophysiology

In order to visualize neuronal somata for targeted patch clamp recordings, cultures were incubated 48h with 1E+9 VG/mL AAV9 expressing EGFP under the neuron specific hSYN promoter 10 days before the experiments. Recordings were performed on an inverted microscope (IX73 Olympus) at room temperature, using a Multiclamp 700B amplifier under control of a Digidata 1550 digitizer board and Clampex 10 software (Molecular Devices), which was also used to trigger the LED. Fluorescent light from an LED (pE-4000, CoolLED) was passed through an appropriate filter (DAPI/FITC/Cy3/Cy5 Quad sbx LED HC Filter Set, AHF) and a 20× dry objective (UPLSAPO20×/0.75 U Plan S Apo). Extracellular solution contained (in mM): 140 NaCl, 2.4 KCl, 10 HEPES, 10 glucose, 2 CaCl2, 1 MgCl2 (pH 7.3, 300 mOsm); intracellular solution contained (in mM): 135 CH3KO3S, 4 NaCl, 2 MgCl2, 10 HEPES, 2 ATP-511 2Na, 0.3 GTP-Na, 0.06 EGTA, 0.01 CaCl2. Whole cell current clamp recordings were performed with bridge balance compensation, and membrane voltage was adjusted manually to <−60 mV if necessary.

### Channelrhodopsin-Stimulation

For specifically stimulating neurons in the neuromuscular culture, cells were incubated with 1E+9 VG/mL AAV8-hSyn-hChR2 (H134R)-GFP for 48 h at day 50. After 3-4 weeks, the expression of ChR2 (H134R)-GFP was assessed using a confocal microscope. Cultures were transferred to the electrophysiology setup described above, and continuously superfused with HEPES-buffered extracellular solution at room temperature. Bright-field images of 2048×2048 were acquired at 10 Hz using an sCMOS camera (Photometrics Prime BSI Express) with 50 ms exposure time. Every 50 s, ChR2-expressing neurons were optically stimulated with three 5-ms flashes of 470 nm light at 10 Hz, with 10 mW/mm2 intensity. For the analysis, the muscle contraction recorded in brightfield was quantified using MuscleMotion (described below), plotted in Prism8 (GraphPad) and correlated to the light 526 pulses.

### Muscle Fiber Orientation Analysis

The orientation of the skeletal muscle fibers was analyzed in control and SMA soNMJ models at day 50. For this, the angle of individual skeletal myofibers was measured in each confocal image. Fast myosin-heavy chain was used as a marker for fiber identification. The resulting measurements from each image were then averaged and the deviation of each measurement from the average was then calculated and plotted in Prism8 (GraphPad). The higher the value, the higher the difference between a single fiber angle and the average value, and the higher the variability of myofibers orientation. Three different images were analyzed in each experiment and three independent experiments were run for each cell line.

Contraction Analysis of soNMJ Model.

Three different regions of the same coverslip were recorded for contraction analysis using a Leica SP8 confocal microscope. Each region was recorded twice for 3 minutes using brightfield with 20× zoom. To quantify the muscle contraction, each movie was analyzed individually using MuscleMotion (an open source ImageJ Macro). A reference frame was automatically detected by MuscleMotion to measure the contraction as a variation in pixel over time. The results were then plotted in Prism8 (GraphPad).

### Quantifications and Statistical Analysis.

Quantifications of day 3 ANPs, NMPs and 2SMADi-treated NMPs were performed with ImageJ using the Cell Counter. The number of SOX2, BRA and TBX6 positive cells was normalized to DAPI. The number of glia cells was quantified as a measure of the total GFAP positive area at day 50 and day 100. For this purpose, a threshold was used to define the total fluorescence area in each image. The same criteria were used to quantify the number of cells expressing LHX3, FOXP1 HOXC6, ISL1, SOX1 and MYF5 and normalized to the total area of DAPI. To quantify the number of NMJs, α-bungarotoxin staining was performed to label AChR clusters. The cells were fixed with 4% PFA and incubated with Alexa Fluor 647 conjugated α-bungarotoxin (Thermo Fisher) for 2 hours. Images were acquired with 40-80× zoom in 3 random locations per sample (n=3-5). To assess cluster number, AChR clusters were quantified for each image and then normalized to the number of Myosin Skeletal Fast positive fibers present in the quantified image. Data are reported as the mean±standard deviation, using a significance level of p<0.05. The number of replicates is indicated in the figure legends. “N” denotes the number of independent experiments and “n” denotes the number of technical replicates/areas analyzed for each sample. A minimum of three random areas were analyzed in each sample. Data were analyzed by one-way and two-way ANOVA, using Bonferroni test for multiple comparisons and Welch's t test for pairwise comparisons (Prism 7-9, GraphPad). Significant differences are indicated as *p≤0.05; ** p≤0.01; *** p≤0.001;**** p≤0.0001.

### Results

Delayed dual SMAD inhibition is necessary for the concurrent development of neural and mesodermal lineages from NMPs. We used five different hPSC lines to explore their potential to generate a self-organizing NMJ (soNMJ) model through an NMP state. We have previously used hPSC-derived NMP cells to generate NMOs in 3D under minimal culture media. However, the direct transfer of the protocol from 3D to 2D was insufficient to generate a functional NMJ model in adherent culture conditions. Analysis of the adherent cultures after applying the NMO conditions at days 20 and 50 revealed the presence of neurons expressing TUBB3 and a few muscle progenitors expressing DESMIN at day 20, which gave rise to only few differentiated skeletal myofibers at day 50. We reasoned that transposition of the 3 dimensional (3D) early NMO culture conditions into two dimensional (2D) may change the autocrine signal signature. BMP inhibition promotes the specification into paraxial mesoderm and skeletal muscles^{2,18} whereas TGFb inhibition further enhances the efficiency of hPSCs differentiation into somites¹⁹. Thus, we employed both BMP inhibition and TGFb inhibition using the herein so termed “dual SMAD inhibitor cocktail” (2SMADi)^{19,20}. We first assessed the effect of 2SMADi at the NMP induction stage (day 0-day 3). We instructed the generation of NMPs using our previously established protocol^{21,22}. Initial exposure of hPSCs to WNT agonist (CHIR) and basic FGF (bFGF) signals for three days resulted in the efficient generation of NMP cells that co-expressed the nascent mesodermal marker BRACHYURY (TBXT) and the neural progenitor marker SOX2. Paraxial mesoderm TBX6+ cells were also evident mainly in the periphery of the NMP colonies. We observed that simultaneous, early treatment of hPSCs with CHIR/bFGF and 2SMADi resulted in the generation of cells that expressed high levels of SOX2 and low levels of TBXT in the absence of TBX6. The low levels of TBXT agreed with the known role of SB431542 to mediate TBXT suppression²³. Thus, addition of 2SMADi changed the balance of TEXT and SOX2 towards a more SOX2 high fate. To determine the effect of 2SMADi in rostro-caudal identity, we analyzed the expression of the posterior markers CDX2, NKX1.2, and HOXB1 and the anterior marker OTX2 in hPSCs treated during days 0-3 with (i) 2SMADi, (ii) WNT/bFGF and (iii) WNT/bFGF/2SMADi. Cells treated with 2SMADi alone acquired an anterior neural identity expressing the neural progenitor markers SOX2, SOX1 and OTX2, indicative of an anterior neural identity and in agreement with previously published studies²⁰.

The WNT/bFGF condition (stimulation) alone resulted in the generation of NMPs that expressed the posterior markers CDX2 and NKX1.2 as previously described^{21,22,24}. The simultaneous addition of 2SMADi and WNT/bFGF resulted in the generation of posterior cells expressing CDX2 and HOXB1. A striking difference between the WNT/bFGF/2SMADi and NT/bFGF treatments was the upregulation of NKX1.2 and SOX2 in the former. This upregulation was associated with a pre-neural tube identity²⁵.

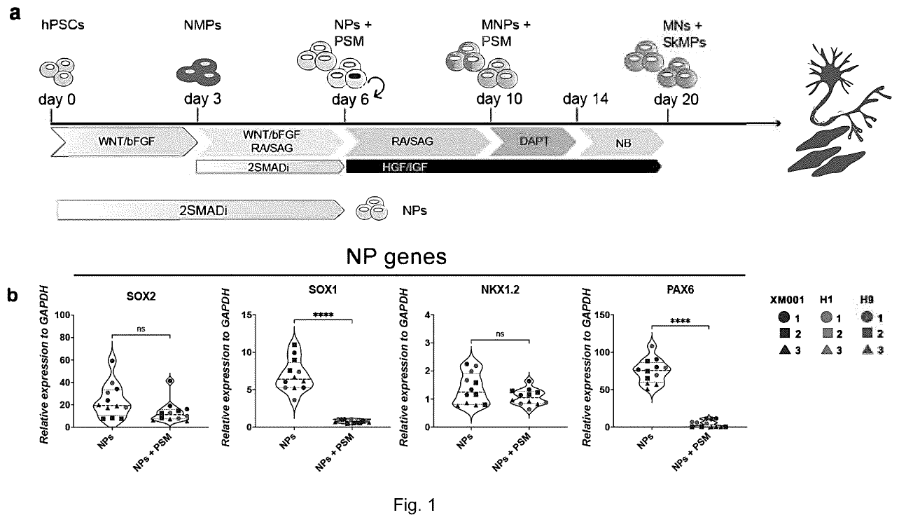

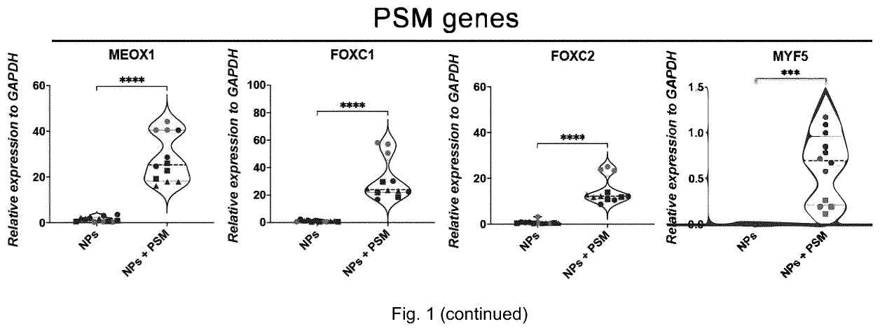

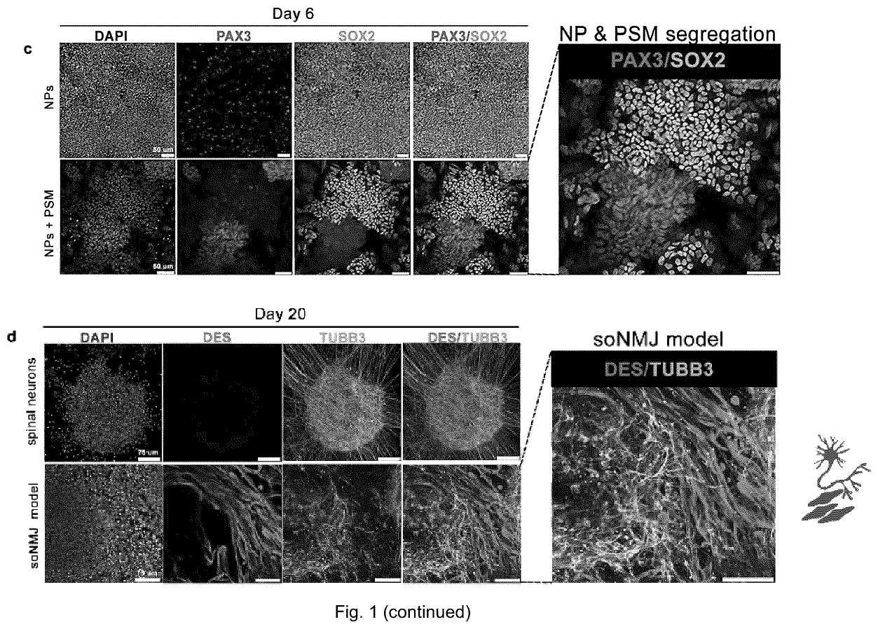

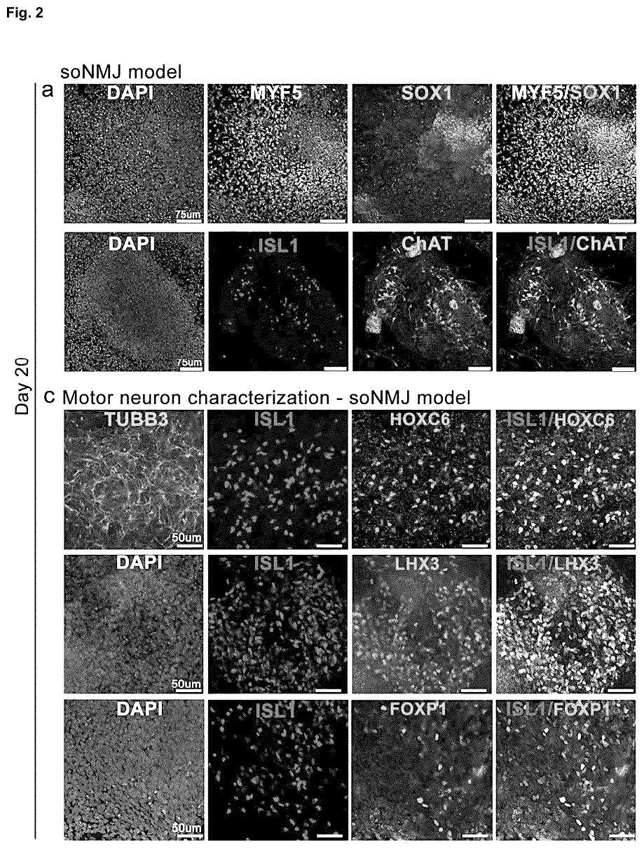

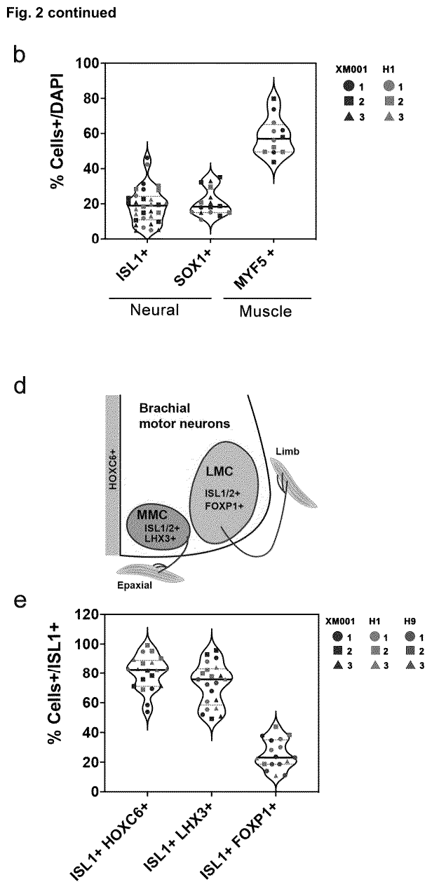

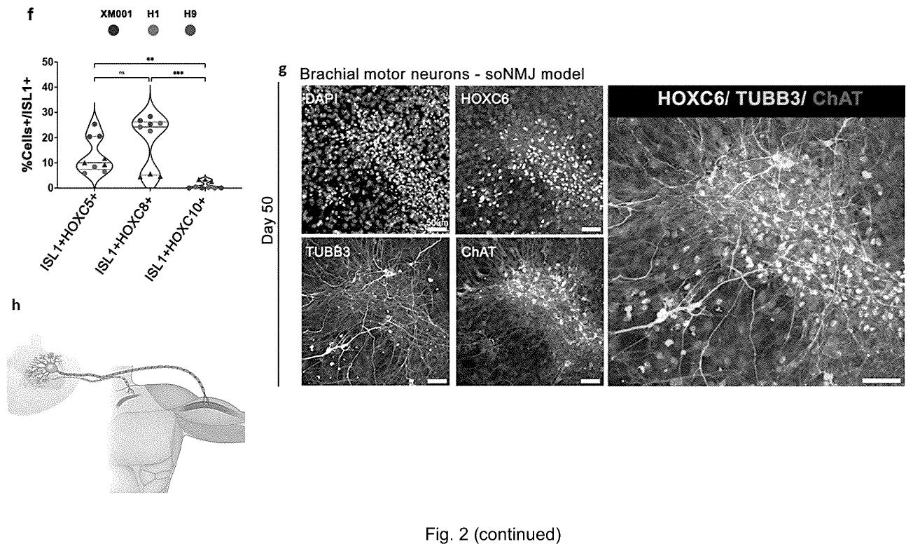

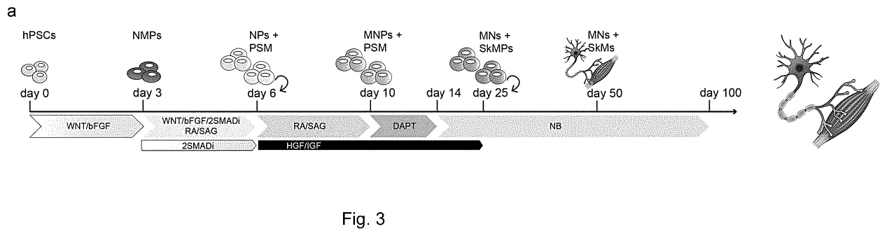

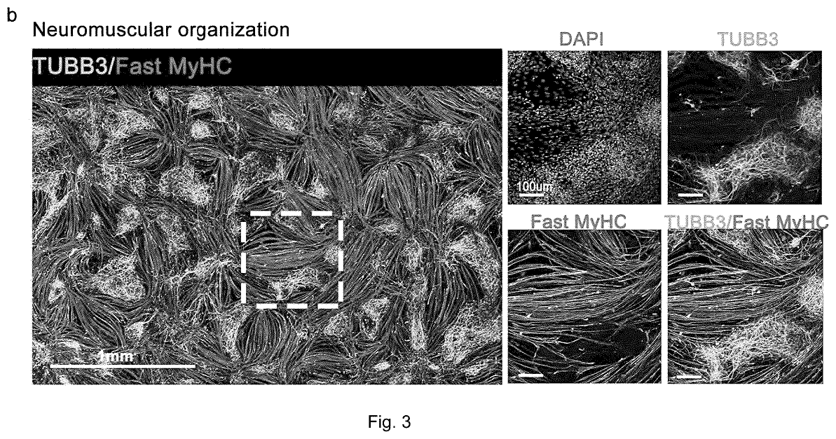

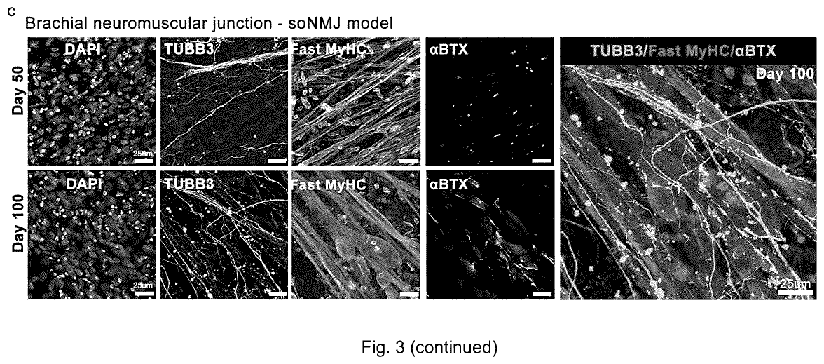

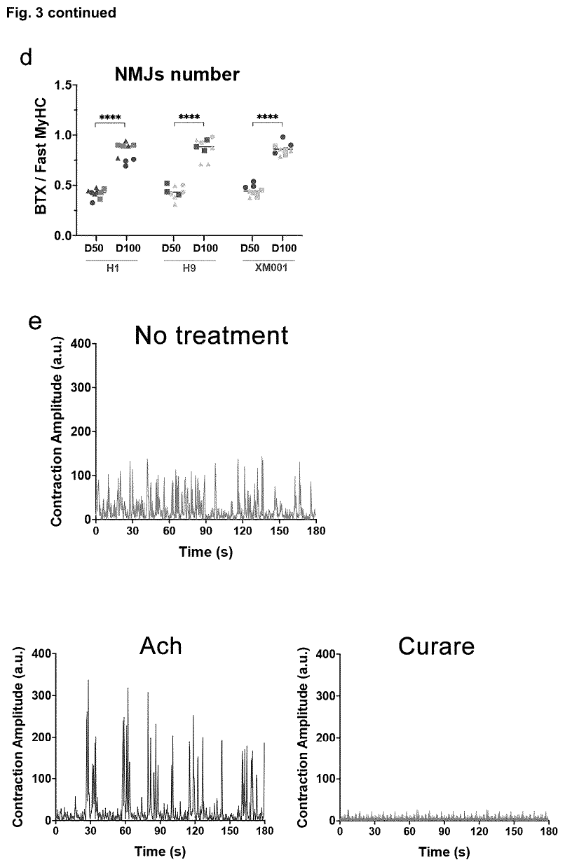

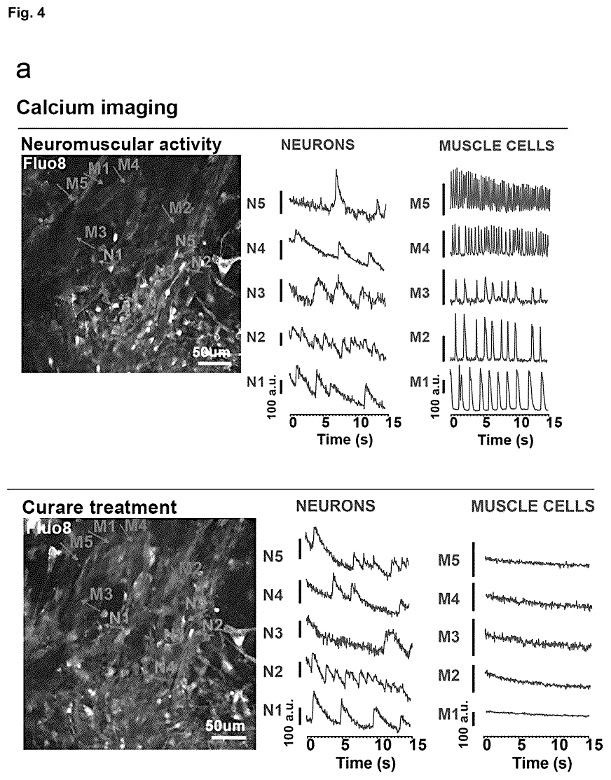

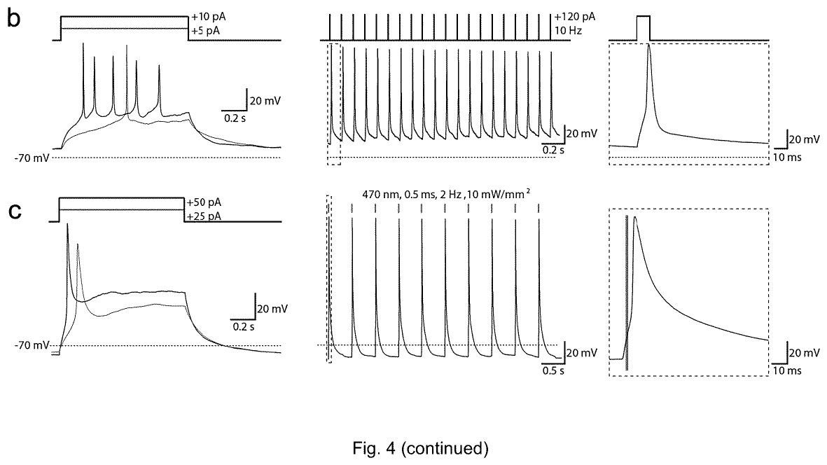

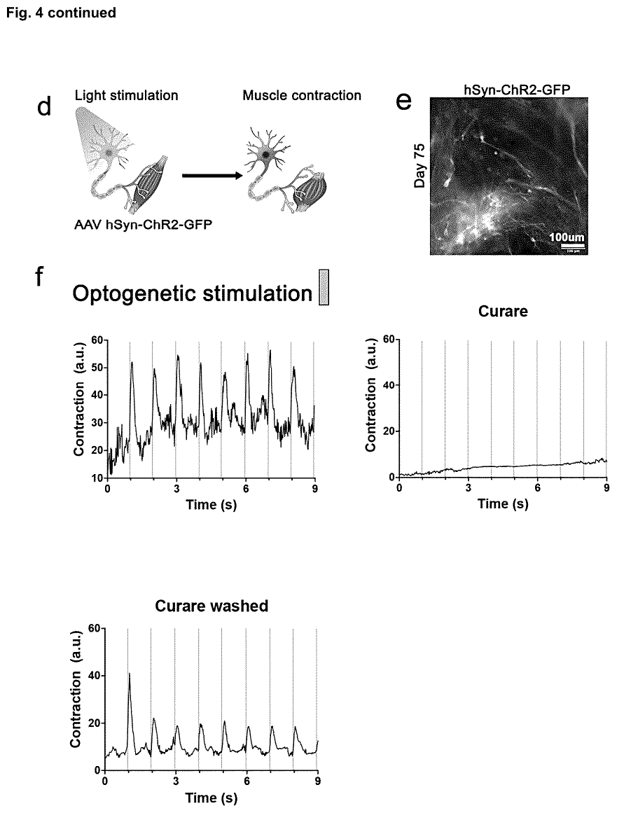

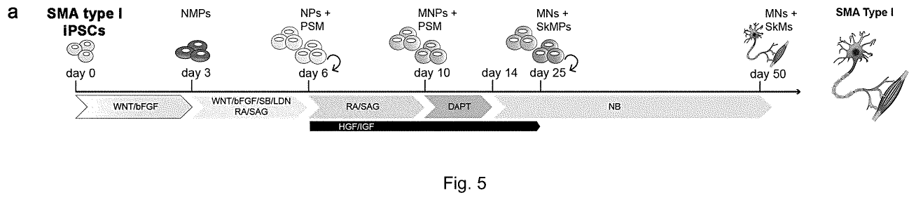

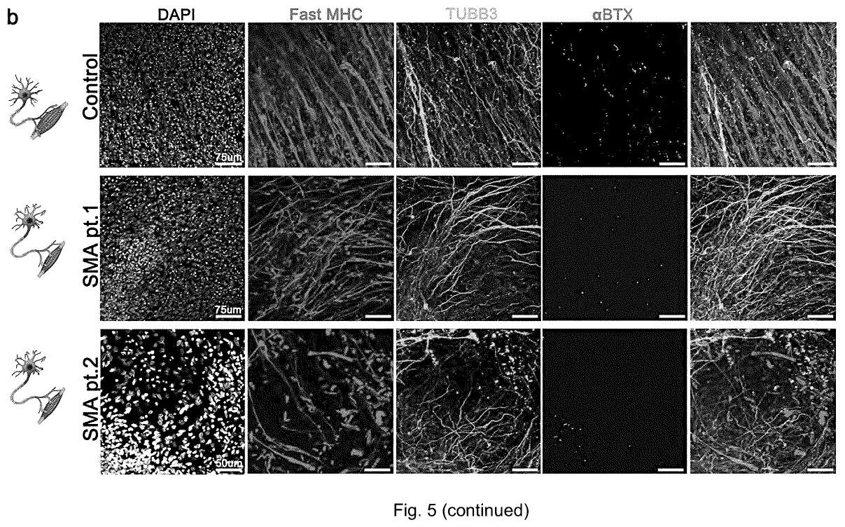

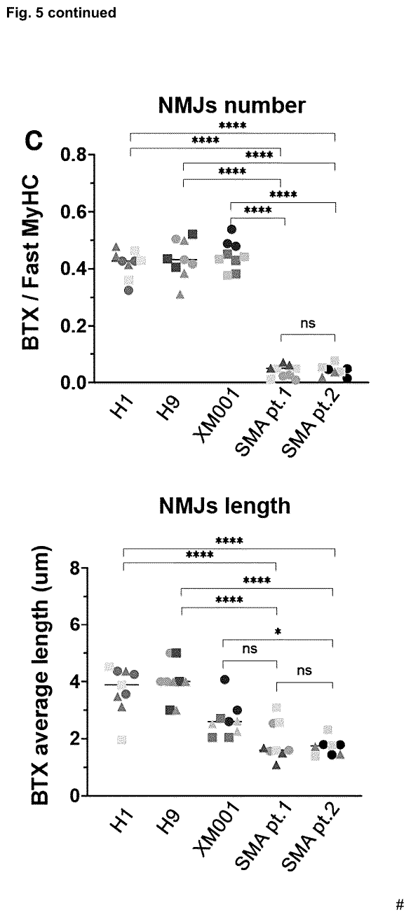

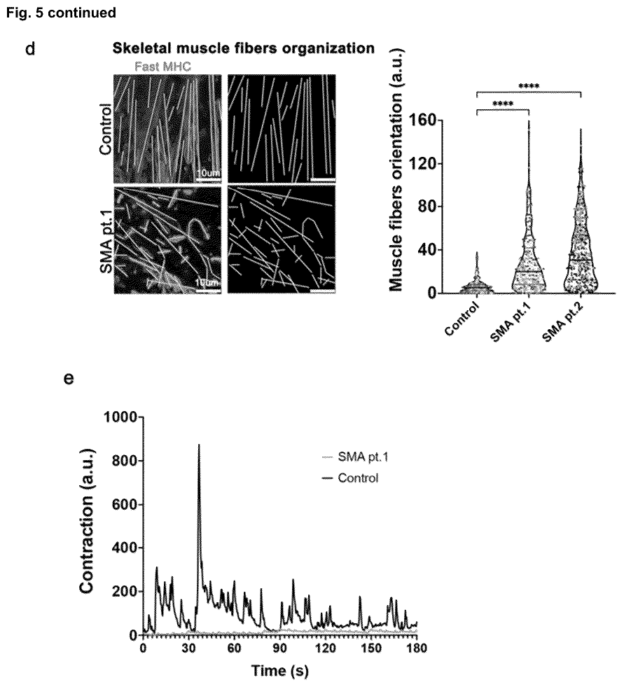

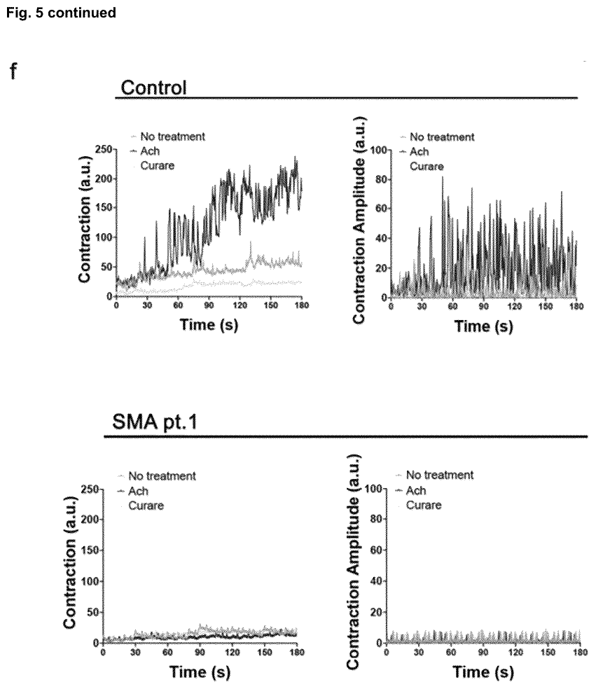

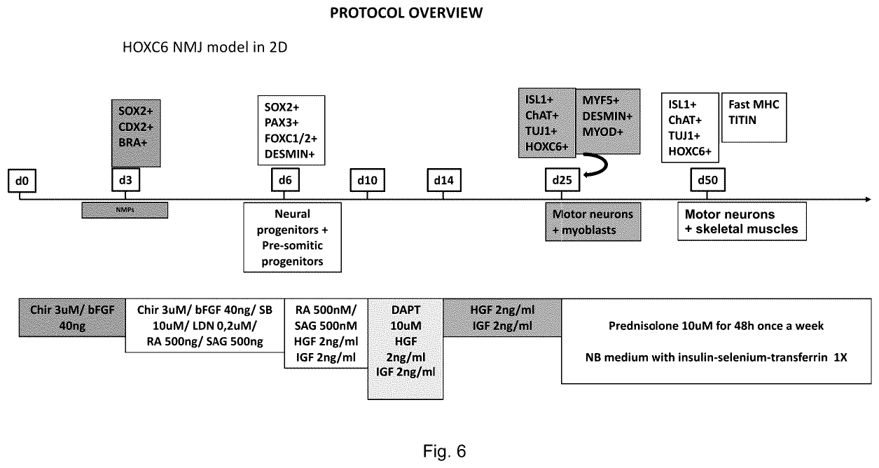

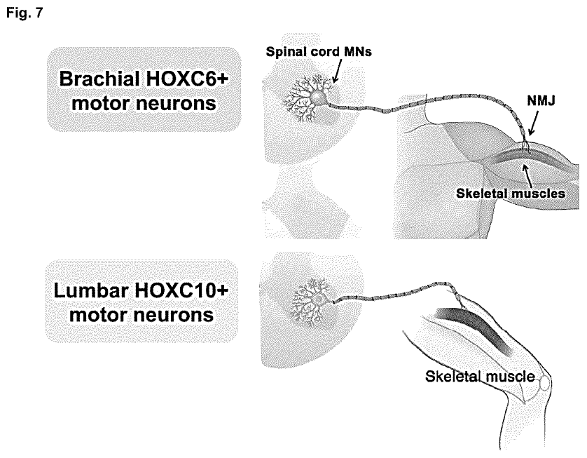

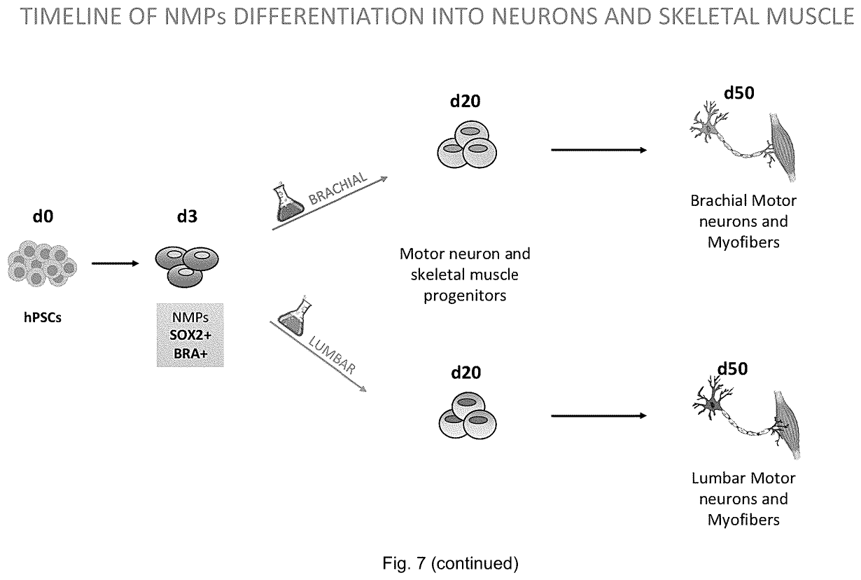

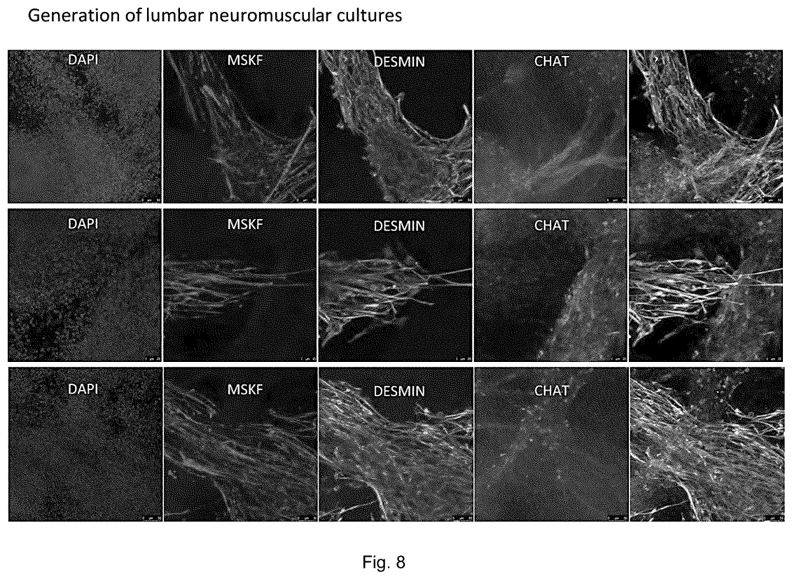

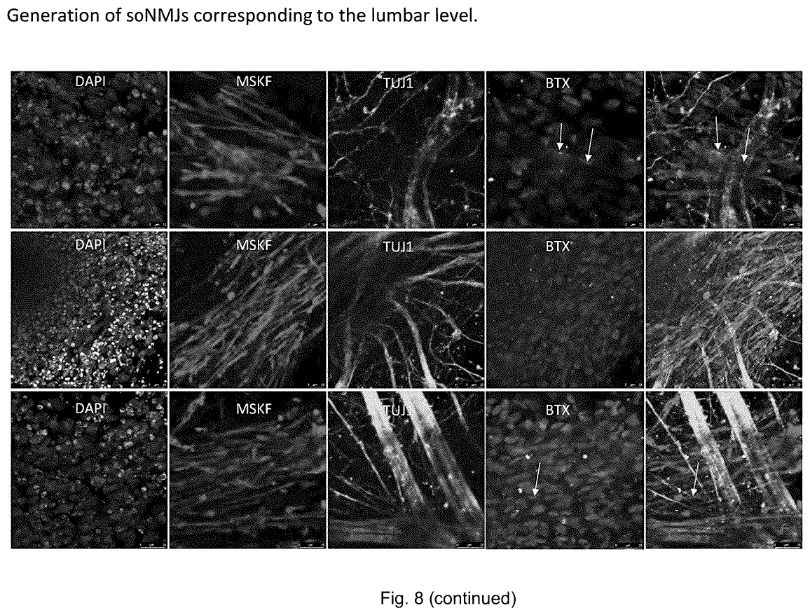

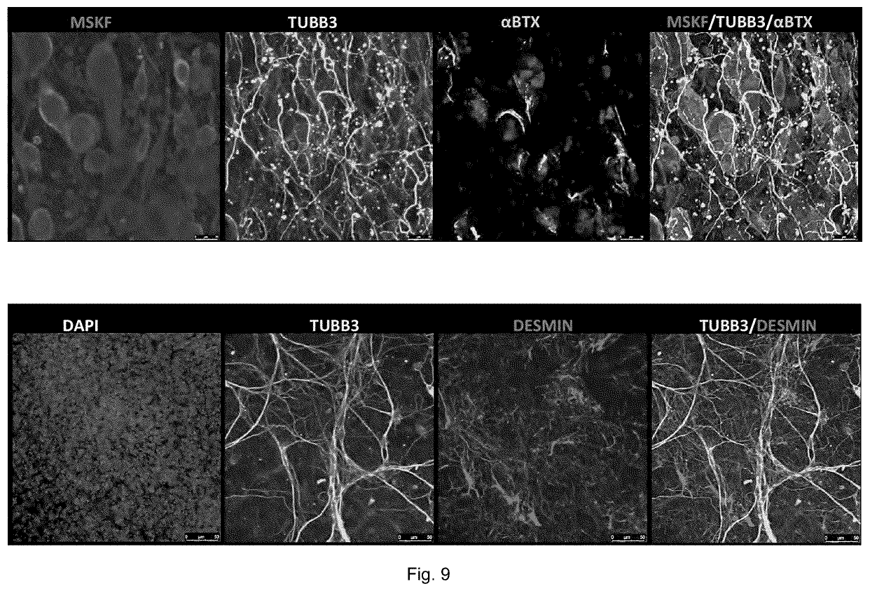

We next examined the potential of the WNT/bFGF/2SMADi treated cells to differentiate into neural and mesodermal progenitors. To enhance the generation of both cell types, we continued the treatment with WNT/bFGF/2SMADi in the presence of retinoic acid (RA) and the SHH smoothened agonist (SAG) to induce a ventral identity for three additional days (FIG. 1a). Analysis of cells on day 6 revealed the exclusive differentiation into neural progenitor cells expressing SOX2, SOX1, PAX6, and NKX1.2 (FIG. 1b, c). Genes associated with a presomitic (PSM) identity, such as MEOX1, FOXC1, FOXC2, and MYF5 were not expressed (FIG. 1b). Thus, exposure of cells to the 2SMADi during the NMP induction stage precludes mesoderm formation. This was in agreement with the finding that 2SMADi promotes the preferential differentiation of hPSCs towards the neuroectodermal lineage^{20,26}. We then explored whether applying 2SMADi after NMP induction could potentiate the simultaneous development and differentiation of neural and mesodermal progenitors (FIG. 1a). Indeed, exposure of NMP cells to WNT/bFGF/2SMADi in the presence of RA and SHH for three days resulted in the generation of clearly segregated neural and mesodermal progenitor clusters that self-organized in the dish (FIG. 1c). The neural progenitor clusters expressed SOX2, whereas the mesodermal clusters expressed PAX3 in the absence of SOX2. Some PAX3+ cells were also evident in the neural SOX2+ clusters corresponding to dorsal neural progenitor identity. PSM specific genes were also upregulated (FIG. 1b).

Thus, we concluded that early treatment of hPSCs with 2SMADi, even in the presence of WNT/bFGF primes NMPs towards an exclusive neural identity, whereas treatment, after the establishment of the NMP state, allows the simultaneous differentiation to both neural and PSM lineages. We next sought to analyze the potential of these cultures to differentiate into spinal cord neurons and skeletal muscle cells. Thus, we passaged the cells at day 6 and plated them in an optimized neurobasal (NB) medium containing 500 nM RA (retinoic acid), 500 nM SAG (SHH smoothened agonist), 2 ng/ml HGF and 2 ng/ml IGF, which could support both the specification of neural progenitor cells towards a ventral MN identity and the proliferation of mesodermal progenitors. RA and SAG were used to pattern the neuroectoderm towards a ventral MN identity^{22,25}. The inclusion of HGF and IGF ensured the survival and proliferation of mesodermal progenitors¹⁸. At day 10, most neural cells expressed the MN progenitor marker OLIG2, at which timepoint progenitors were treated with the γ-secretase inhibitor DAPT to accelerate the generation of MNs 4. Analysis on day 14 revealed the presence of both OLIG2+ cells and HB9+ differentiated MNs. After day 14, cells were maintained in the presence of only HGF and IGF. Comparison of the expression profile of genes associated with neural and mesodermal differentiation between day 3, day 6, and day 20 confirmed the differentiation of NMP cells to neural progenitors that expressed SOX2, SOX1, OLIG2, and more differentiated MNs that expressed ISL1, FOXP1, and CHAT. As expected, on day 20, the expression of the muscle-specific progenitor markers MYF5 and MYOD was upregulated. In contrast, the expression of the early mesodermal marker MSGN1 was completely downregulated. The early segregation of NP and PSM clusters, observed at day 6, was maintained and developed into clusters of differentiated neural TUBB3± cells and muscle DESMIN+ progenitors at day 20 (FIG. 1d). Quantitative analysis of the neural and muscle-specific cell types revealed the presence of 18.9%±10.1% ISL1+ cells, 20.9%±7.6% SOX1+ and 58.4%±10.7% MYF5+ cells (FIG. 2a,b). Collectively, the data showed that 2SMADi during the NMP induction stage effectively eliminates the mesodermal lineage giving rise to posterior neurons. In contrast, delayed 2SMADi exposure, after the induction of NMP state, promotes the simultaneous development and differentiation of both neural and mesodermal lineages. Then, cells self-organize into distinct clusters of differentiated neurons and muscle progenitors. Thus, fine-tuning the timing of 2SMADi exposure allows the selective differentiation to either exclusively posterior neurons or neuromuscular cells. Efficient generation of a position-specific soNMJ model that includes supporting glial cells Generating position-specific spinal cord neurons is necessary for proper disease modeling and transplantation studies. Thus, we next sought to characterize the rosto-caudal and columnar identity of the spinal cord neurons in this neuromuscular model. It is well established that the identity of spinal cord neurons is instructed by the differential expression of HOX genes²⁷. To define the rostrocaudal identity, we examined the expression of HOX proteins at day²⁰. Most of the MNs co-expressed HOXC6/ISL1 (80.2%±11.9%), confirming their brachial spinal cord identity (FIG. 2c,e). We then assessed the acquisition of a defined columnar subtype identity. At the brachial spinal cord level, MNs are organized into two columnar identities corresponding to the lateral motor column (LMC) and median motor column (MMC) (FIG. 2d) 27. Analysis with specific columnar identity markers revealed that approximately 80% of the MNs co-expressed ISL1/LHX3 (72.4%±14.4%), associated with MMC identity while 20% co-expressed ISL1/2/FOXP1 (25.3%±10%) associated with LMC identity (FIG. 2c,e). Consistent with continuing maturation, at day 50, brachial spinal cord MNs were expressing the acetylcholine-synthesizing enzyme choline acetyltransferase (ChAT) and HOXC6, whereas the expression of the early MN marker ISL1/2 was completely abolished (FIG. 2f)²⁸.

It is worth noting that the MNs survived and matured in the neuromuscular model without external supplementation of neurotrophic factors. Maturation of MNs in reductionist models depends on the addition of a medium enriched for glial-derived neurotrophic factor (GDNF), brain-derived neurotrophic factor (BDNF) as well as ciliary neurotrophic factor (CNTF)^{4,28}. However, this is not required in this neuromuscular model, probably due to the presence of muscle and glial cells that support the MNs. GFAP+ glia cells were first detected on day 50 without adding factors that support gliogenesis or serum²⁹. This suggested that in the neuromuscular model neural progenitor cells can be maintained long enough to make the gliogenic switch. Indeed, the number of glial cells significantly increased from day 50 to day 100, recapitulating the later developmental timing of glial cells in vivo. Additionally, day 50 immunostainings revealed the presence of V2a excitatory pre-motor interneurons, marked by expression of CHX10 and V1, V0, and dl6 interneurons marked by PAX2/LHX1 expression. Therefore, timely application of the appropriate instructive and mitogenic signals until day 25 generates a neuromuscular model of defined rostrocaudal and columnar identity (FIG. 3a). Then, the co-developing cell types provide the necessary autocrine and paracrine signals giving rise to a dynamic, self-instructing model system that develops and matures with minimal requirements for exogenously added differentiation and maturation factors. High-content imaging reveals self-organization of spinal cord neurons and skeletal muscles to form maturing neuromuscular junctions. Neuromuscular cultures were very dense by day 25-30, and we assessed whether cells would maintain their self-organizing capacity after passaging.

Cultures were analyzed at day 50 using a high-content imaging system that allowed us to capture the whole culture dish. Immunofluorescence analysis with Myosin skeletal fast (Fast MHC) and TUBB3 revealed the organization of skeletal myofibers in bundles surrounded by spinal cord neurons that could be maintained in culture for more than 100 days (FIG. 3a,b). 3D projection of the neuromuscular images revealed that, along the z-axis, the muscles and neurons were also segregating on different layers, confirming that this is a self-organizing NMJ (soNMJ) model.

It was recently shown that skeletal myofibers self-organized in bundles instructed by mechanical tension in 2D cultures 32. Still, in those cultures, the contraction of the skeletal muscles could not be maintained for too long due to loss of attachment. An important advantage of the soNMJ model is that muscles remained attached to the dish for long periods, even under contractile conditions. At day 50, we detected numerous acetylcholine receptor (AChR) clusters by staining for a-bungarotoxin (aBTX) contacted by TUBB3+ neurites, suggesting the formation of NMJs.

To address the reproducibility of the soNMJ model, we analyzed the number of NMJs in three different PSC lines at day 50 and day 100 (FIG. 3c,d). The number of NMJs was similar among the different lines at day 50 when normalized to the total number of muscle fibers. In agreement with a stepwise, ongoing maturation, the number and size of the NMJs significantly increased from day 50 to day 100 (NMJs length >5 pm) (FIG. 3c,d).

At day 100, the skeletal muscle fibers were multinucleated and had the typical striation of sarcomere organization. A characteristic of mature myofibers is the presence of PAX7+ muscle stem cells, also known as satellite cells. At day 100, we observed PAX7+ cells underneath the basal lamina, an arrangement reminiscent of satellite cells in vivo 33. Apart from MNs and skeletal muscles, terminal Schwann cells are necessary for the maturation and stability of NMJs.

Among the different cell types originating from NMPs, there are trunk neural crest cells that generate terminal Schwann cells. Thus, we assessed the presence of terminal Schwann cells in the soNMJ culture using the expression of the S10013 epitope at the NMJ. At day 50, S100beta⁺ Schwann cells were already present and by day 100, they could be found at the NMJs, located between the AChR clusters and the neural axons.

Collectively, these data suggested that brachial MNs interacted with the skeletal myofibers to form NMJs supported by terminal Schwann cells. The increase in the number and size of the NMJs over time, as well as the presence of multinucleation and striation in the myofibers, suggested that the neuromuscular model matured over time. This was further supported by the detection of PAX7+ satellite-like cells and S100beta⁺ terminal Schwann cells. Functional analysis of the soNMJ model. To investigate the functionality of the soNMJs, we analyzed the contractile muscle activity at day 75 after treatment with 10 μM acetylcholine and after treatment with 10 pM curare, which is a blocker of the AChR. Treatment with acetylcholine accelerated the contraction of skeletal muscles. In contrast, treatment with curare blocked the contraction of the skeletal muscles (FIG. 3e). We further characterized the activity of the neuromuscular cultures using calcium imaging and optogenetic stimulation. To record the calcium activity, we used the ratiometric fluorescent calcium-binding dye Fluo8. Calcium transients were observed in spinal neurons and skeletal muscles that were distinguishable by speed and morphology (FIG. 4a). Treatment with curare abolished the calcium activity in the skeletal muscles without affecting the firing of the neurons (FIG. 4a).

To check the functionality of the spinal neurons, we used patch-clamp to record the electrical activity. Neuronal somata were visualized by AAV-mediated neuron-specific expression of nucleus-targeted EGFP. Neurons had a resting membrane potential of −61.94±5 mV (n=5, corrected for liquid junction potential) and a low capacitance (18.72±1.37 pF). Injection of 1-s long depolarizing currents elicited single or multiple action potentials (FIG. 4b), demonstrating functional neuronal physiology. Moreover, repetitive injections of short (10 ms) depolarizing currents at 10 Hz reliably elicited trains of action potentials (FIG. 4c). In ChR2-expressing neurons, we found that very brief light flashes could elicit similar repetitive firing (FIG. 4c). To obtain optogenetic control of neuronal firing, we transduced day 50 cultures with an adeno-associated virus (AAV) in which the expression of Channelrhodopsin2 (ChR2)-GFP is under the control of the human SYNAPSIN promoter. After 3-4 weeks, we performed whole-cell current clamp recordings on ChR2-GFP-expressing neurons and found that trains of brief light flashes could elicit repetitive firing (FIG. 4c). The selective expression of ChR2 in spinal neurons allowed us to optogenetically stimulate synchronous neuronal firing, and thus to test for light-induced muscle contraction driven by synaptic transmission (FIG. 4d). At day 75, spinal neurons showed robust ChR2-GFP expression (FIG. 4e). Light stimulation of these cultures instructed the synchronous contraction of the skeletal muscles providing concrete evidence of controllable NMJ function. The addition of curare blocked the optogenetic stimulation of the skeletal muscles, further supporting our findings (FIG. 4f). The analysis revealed the presence of spinal cord neurons and skeletal muscles forming functional networks. This defined protocol takes advantage of the co-development of the neural and mesodermal lineages and their self-instructing and self-organizing attributes. The result is an efficient and robust platform for rapidly generating hPSC-derived functional soNMJs in 2D that could be used for disease modeling and high throughput studies. The soNMJ model as a platform to study Spinal Muscular Atrophy Spinal muscular atrophy (SMA) is a disease mostly caused by deletions in the SMN1 gene locus that lead to reduced levels of SMN protein³⁵. While humans carry nearly identical copies of the SMN1 gene (SMN2) on the same chromosome, a base change in a splice site in exon 7 leads to insufficient expression of the full-length transcript³⁶. SMN2 exists in variable number of copies (2-8) which determines the severity of the disease³⁷. SMA has been initially considered a MN specific disease, but animal studies suggest an earlier effect at the NMJ and skeletal muscle³⁸⁻⁴². Notably, the loss of the MN population usually only occurs at the end stages of the disease, and it might be a consequence of structural and functional defects at the NMJs that are the earliest pathological changes detected in SMA mouse models⁴³⁻⁴⁶.

To provide a proof of concept for our soNMJ model, we used human induced pluripotent stem cells (hiPSCs) from two patients (materials and methods) with SMA to generate soNMJs (FIG. 5a). Both patients had a severe form of type 1 SMA with only two copies of SMN2. Due to the high presence of repetitive elements in the genomic locus of SMN1, it was not possible to target the deleted sequence to generate an isogenic line and thus we compared them to well characterized control PSC lines. Both patient iPSC lines were first analyzed for the expression levels of SMN protein by immunofluorescence, which revealed a reduction in SMN levels compared to control.

We further used the SMA lines to generate soNMJs in vitro through an NMP intermediate state. The SMA neuromuscular cultures were analyzed at the progenitor (day 20) and mature state (day 50) for the presence of neural and muscle-specific progenitor cells and the formation of NMJs (FIG. 5a). At day 20, DESMIN+ myoblast cells and SOX1+NPs were detected in both the SMA type I and control hPSC lines. However, at this stage, SMA myofibers were already smaller and less elongated in the SMA type I derived soNMJs. This suggested a delay in the differentiation and maturation of the skeletal muscle cells. At day 20, ChAT+ MNs were also present in both SMA and control cultures but without detectable morphological differences. This suggested that the MNs were not affected at this point.

Next, we analyzed the cells at day 50 for the presence of mature Fast MHC+ skeletal myofibers and TUBB3+ neurons (FIG. 5b). At this stage, the SMA myoblasts were smaller and showed reduced elongation and organization (FIG. 5b). Additionally, at day 50, SMA myoblasts generated Fast MHC±myofibers that were reduced in length, disorganized, pointing at different directions instead of aligning, and failing to form bundles (FIG. 5d). These results strongly suggested an impaired maturation of the skeletal muscle cells in SMA. Immunofluorescence analysis revealed that ChAT±MNs were present in both SMA and control lines. Unexpectedly, the abundance of MNs appeared higher in SMA than in control conditions. This may reflect a bona fide aspect of early SMA pathology corresponding to the asymptomatic phase of the disease and it might be due to a longer proliferation phase resulting from differentiation failure. Synaptic defects at the NMJs are one of the hallmarks of SMA pathology⁴³. We analyzed the presence of AChR clusters to investigate whether this phenotype was reproduced in the SMA soNMJ model. Quantification of the alphaBTX±clusters showed that, in SMA, NMJs were significantly reduced in number and size compared to the hPSC lines H9, H1 and XM001 (FIG. 5b, c). Analysis of muscle contraction showed the presence of short-duration weak movements in individual muscle fibers, followed by exhaustion and a complete lack of activity. Thus, the contractility of the SMA skeletal myofibers was severely impaired (FIG. 5e). We subsequently tested if addition of acetylcholine would stimulate the muscle contraction and whether the administration of curare would block the spontaneous residual muscle contractility. Acetylcholine administration did not stimulate contraction, whereas curare failed to stop spontaneous muscle activity, suggesting the lack of functional NMJs in the SMA model (FIG. 5f). Collectively, these data reveal an apparent impairment in the formation of synapses between the MN endplates and the skeletal muscle in SMA prior to MN cell death. Thus, the soNMJ model provides unique opportunities for mechanistic studies that will elucidate the sequence of events leading to neuromuscular diseases in humans.

### Discussion

Here, we present a bottom-up approach to generate functional self-organizing NMJs in vitro from hPSCs. We show that the combinatorial and temporal application of small molecules supports the initial patterning of neural and mesodermal cell types from hPSCs through an NMP state. Consistent with previous studies, early exposure of hPSCs to 2SMADi restricts the potential of NMP cells to generate mesoderm and instructs them toward an exclusively neural spinal cord identity^{5,26}. Strikingly, exposure to 2SMADi after the NMP state supported the growth and differentiation of spinal cord neurons and skeletal muscles that self-organized to generate functional soNMJs. Surprisingly, fine-tuning the exposure time to 2SMADi is sufficient to generate either spinal neurons or a complete brachial soNMJ model from hPSCs. An important advantage of the soNMJ model over co-culture models is that it can be maintained for more than 100 days and that NMJs form and mature during this time in the absence of exogenous stimuli. This supports the notion that co-developing cell types provide the necessary support that gives rise to a dynamic, self-instructing model system that develops and matures with minimal requirements for exogenously added differentiation and maturation factors.

To our knowledge, this is the first position specific NMJ model that can self-organize and be maintained for extended periods of time under adherent cell culture conditions.

Furthermore, the contraction of the skeletal muscles, in the soNMJ model, is instructed by the MNs and persists for long periods of time without the detachment issues that affect other models^{11,47}. Analysis of the rostrocaudal identity revealed the generation of brachial MNs corresponding to both median and lateral motor columns. This demonstrates the feasibility of generating position-specific NMJ models from hPSCs.

The development of position-specific NMJ models will open up the exciting opportunity to study the selective vulnerability of position-specific neurons to neuromuscular diseases. The establishment of the soNMJ model offers an efficient, defined, and robust platform that can be used for mechanistic studies and high throughput approaches. The simplicity and scalability of the soNMJ model is ideal for drug screening studies and development of novel therapeutic approaches for neuromuscular diseases, paving the road to personalized medicine.

### Example 2

The following protocol was performed as an exemplary embodiment to generate a soNMJ model.

On day 0 and before the start of the experiment culture dishes were pre-coated with Matrigel and the necessary amount of medium for NMPs induction was prepared as follows (calculate 1.5 mL/p35).

### NMPs Induction Medium: N2B27*+

3 μM CHIRON/CHIR99021/Chir (Highly selective small molecule GSK-3 inhibitor; acts as Wnt activator)
40 ng/μL bFGF
10 μM Rock Inhibitor (RI)

The necessary amount of Accutase was prepared, and NB media was prepared freshly with the small molecule GSK-3 inhibitor (every day).

The N2B27 medium comprised: NB/DMEM-F12 1:1, N2, B27, Glutamate, P/S and B-Mercaptoethanol.

The following protocol was performed on day 0 (D0):

hESCs Dissociation and Plating:
X1 Wash PBS (1 mL/well)
Add 1 mL/well Accutase
Incubate at 37° C. for 2-3 min until ESCs start to lift,
Add 1 mL NB medium/well to stop the reaction,
Pipette gently up and down with a P1000 to create an homogeneous single cell suspension
Centrifuge 4′ at 600 g
Resuspend the pellet with a right amount of NMPs induction medium for counting (normally 1 mL every 3-4 wells of a 6 wp is enough to have a good suspension)
Count cells with Trypan Blue
Plate a density of viable cells of 10×105 cells/cm² in a p35 dish (around 7.5-8×105 cells/p35)
Immediately add 1.5 mL NMPs induction medium/p35 and distribute the cells homogeneously around the dish,

NB: the plating numbers have been optimized with 5 different hES/hiPS cells lines, including H9 and H1. However, if the d1 culture is too much or too less confluent, the numbers should be adjusted.

The protocol of day 1 (D1) and day 2 (D2) was in brief:

Medium change:
Prepare the necessary amount of NMPs medium (calculate 1.5 mL/p35): NMPs Medium: N2B27+
3 μM CHIR99021 (GSK-3 inhibitor; acts as Wnt activator)
40 ng/μL bFGF

NB media: on D1 change medium preferentially in the morning, to not leave the cells too long in RI. Prepare the medium with the GSK-3 inhibitor small molecule inhibitor fresh every day.

The protocol of day 3-5 (D3-5) was in brief:

### Medium Change:

Prepare the necessary amount of neural progenitors and pre-somitic mesoderm medium (NP+PSM) medium (calculate 1.5 mL/p35):

NP+PSM Medium: N2B27+
3 μM CHIR99021
40 ng/μL bFGF
10 μM SB431542 (SB; inhibitor of transforming growth factor-β receptor (TGF-βR))
0.2 μM of a selective BMP signaling inhibitor, e.g., LDN193189
500 nM RA (retinoic acid)
500 nM SAG (SHH smoothened agonist)

NB: change medium every day. On D6 the culture needs to be replated: the mesoderm forms balls of tissue that otherwise would detach from the dish (some detachment is normal and it will disappear after replating).

The protocol of day 6 (D6) was in brief:

### Replating:

### A. Before Starting:

Pre-coat culture dishes with Matrigel: use 35 mm IBIDI dishes with 1.5 kPa stiffness
Prepare the necessary amount of medium for motor neuron progenitors and myoblasts (MNP+myoblast) induction (calculate 1.5 mL/p35 and 1 mL/well for a 12 wp with coverslips):

### MNPs+Myoblast Induction Medium:

N2B27+
RA 500 nM
SAG 500 nM
HGF 2 ng/ml
IGF 2 ng/ml
Rock Inhibitor (RI) 10 μM

Prepare the necessary amount of Accutase.

B. Replating into IBIDI Plates:
X1 Wash PBS (1 mL/well),
Add 1 mL/well Accutase,
Incubate at 37° C. for 3 minutes,
Add 1 mL NB medium to stop the reaction,
Pipette gently up and down with a P1000 to create an homogeneous single cell suspension
Centrifuge 4′ at 600 g,
Resuspend the pellet with a right amount of NMP+myoblast induction medium for counting (normally 2 mL every p35 is enough to have a good suspension),
Count cells with Trypan Blue,
NB: generally around 7×106 cells are expected from one p35 dish on D6,
Plate a density of viable cells of 10×105 cells/cm2 in an IBIDI dish (around 7.5-8×105 cells/p35), and 4.5×105 cells/cm2 in a well of a 12 wp with coverslips (around 1.8×105 cells/well) in case needed.
Immediately add 1.5 mL/IBIDI or 1 mL/well of 12 wp of NMP+ myoblast induction medium and distribute the cells homogeneously around the dish/well.

NB medium: The last step needs to be fast, in order to have the cells nicely distributed in the plate before they start to settle down. The plating numbers have been optimized with 5 different hES/hiPS cells lines, including H9 and H1. However, if the d7 culture is too much or too less confluent, the numbers should be adjusted.

The protocol of day 7 (D7) was in brief:

### Medium Change:

Prepare the necessary amount of MNPs medium (calculate 1.5 mL/p35 and 1 mL/w12 wp):

### MNP+Myoblast Medium:

N2B27+
RA 500 nM
SAG 500 nM
HGF 2 ng/ml
IGF 2 ng/ml

NB medium: Change medium every day.

The protocol of day 10-13 (D10-13) was in brief:

### Medium Change:

Prepare the necessary amount of motor neuron (MN) medium (calculate 1.5 mL/p35 and 1 mL/w12 wp):

### MN Medium:

N2B27+
DAPT 10 μM
HGF 2 ng/ml
IGF 2 ng/ml

NB medium: Change medium every day.

The protocol of day 14-25 (D14-25) was in brief:

### Medium Change:

From now on the medium can be changed every other day.
Prepare the necessary amount of motor neuron (MN) maintenance medium (calculate 1.5 mL/p35 and 1 mL/w12 wp):

### MN+Myoblast Maintenance Medium:

N2B27+
HGF 2 ng/ml
IGF 2 ng/ml

NB medium: at day 25 the cultures become very dense and need replating.

The protocol of day 25 (D25) was in brief:

### Replating:

### A. Before Starting:

Pre-coat culture dishes with Matrigel,
Prepare the necessary amount of medium for MN+skM medium (calculate 1.5 mL/p35 and 1 mL/well for a 12 wp with coverslips):

### MN+skM (Motor Neuron+Skeletal Muscle) Medium:

N2B27 medium
HGF 2 ng/ml
IGF 2 ng/ml
Rock Inhibitor (RI) 10 μM
ITS 1×
Prednisolone 10 μM

Prepare the necessary amount of Accutase. Prepare the necessary amount of TryPLE express.

### B. Replating:

X1 Wash PBS (1 mL/well),
Add 1 mL/well TryPLE express,
Incubate at 37° C. for 3 minutes,
Aspirate TryPLE expressed and wash once with warm NB, then aspirate the medium,
Add 1 mL of final MN+skM medium with RI inside,
Pipette gently up and down with a P1000 to break the cells into clumps,
Distribute the cells into Matrigel-coated dishes. From 1 IBIDI you can plate: ×1 12-wp, or ×1 24-wp with Matrigel-coated coverslips. Distribute the clumps evenly and incubate at 37° C.

The protocol of day 26-28 (D26-28) was in brief:

### Medium Change:

Maintain the cells in MN+SkM (motor neuron+skeletal muscle) medium.

Important: changing medium every day is absolutely essential in this step.

### After Day 25

Medium change:
Maintain the cells in MN+SkM maintenance medium.
MN+skM (motor neuron+skeletal muscle) maintenance medium: N2B27+
ITS 1×
Prednisolone 10 μm: use only once per week.

From now on increase the volume in each culture dish: 1.5 mL for each well of a 12-wellp.

If cultures get very yellow increase to 2 mL. Change medium very other day.

### REFERENCES

1. Sances, S. et al. Modeling ALS with motor neurons derived from human induced pluripotent stem cells. Nat Neurosci 19, 542-553, doi: 10.1038/nn.4273 (2016).
2. Chal, J. et al. Generation of human muscle fibers and satellite-like cells from human pluripotent stem cells in vitro. Nat Protoc 11, 1833-1850, doi: 10.1038/nprot.2016.110 (2016).
3. Chal, J. et al. Differentiation of pluripotent stem cells to muscle fiber to model Duchenne muscular dystrophy. Nat Biotechnol 33, 962-969, doi: 10.1038/nbt.3297 (2015).
4. Maury, Y. et al. Combinatorial analysis of developmental cues efficiently converts human pluripotent stem cells into multiple neuronal subtypes. Nat Biotechnol 33, 89-96, doi: 10.1038/nbt.3049 (2015).
5. Mouilleau, V. et al. Dynamic extrinsic pacing of the HOX clock in human axial progenitors controls motor neuron subtype specification. Development 148, doi: 10.1242/dev.194514 (2021).
6. Wind, M. & Tsakiridis, A. In Vitro Generation of Posterior Motor Neurons from Human Pluripotent Stem Cells. Curr Protoc 1, e244, doi: 10.1002/cpz1.244 (2021).
7. Pourquie, O., Al Tanoury, Z. & Chal, J. The Long Road to Making Muscle In Vitro. Curr Top Dev Biol 129, 123-142, doi: 10.1016/bs.ctdb.2018.03.003 (2018).
8. Maffioletti, S. M. et al. Three-Dimensional Human iPSC-Derived Artificial Skeletal Muscles Model Muscular Dystrophies and Enable Multilineage Tissue Engineering. Cell Rep 23, 899-908, doi: 10.1016/j.celrep.2018.03.091 (2018).
9. Osaki, T., Uzel, S. G. M. & Kamm, R. D. Microphysiological 3D model of amyotrophic lateral sclerosis (ALS) from human iPS-derived muscle cells and optogenetic motor neurons. Sci Adv 4, eaat5847, doi: 10.1126/sciadv.aat5847 (2018).
10. Vila, O. F. et al. Quantification of human neuromuscular function through optogenetics. Theranostics 9, 1232-1246, doi: 10.7150/thno.25735 (2019).
11. Afshar Bakooshli, M. et al. A 3D culture model of innervated human skeletal muscle enables studies of the adult neuromuscular junction. Elife 8, doi: 10.7554/eLife.44530 (2019).
12. Steinbeck, J. A. et al. Functional Connectivity under Optogenetic Control Allows Modeling of Human Neuromuscular Disease. Cell Stem Cell 18, 134-143, doi: 10.1016/j.stem.2015.10.002 (2016).
13. Demestre, M. et al. Formation and characterisation of neuromuscular junctions between hiPSC derived motoneurons and myotubes. Stem Cell Res 15, 328-336, doi: 10.1016/j.scr.2015.07.005 (2015).
14. Santhanam, N. et al. Stem cell derived phenotypic human neuromuscular junction model for dose response evaluation of therapeutics. Biomaterials 166, 64-78, doi: 10.1016/j.biomaterials.2018.02.047 (2018).
15. Picchiarelli, G. et al. FUS-mediated regulation of acetylcholine receptor transcription at neuromuscular junctions is compromised in amyotrophic lateral sclerosis. Nat Neurosci 22, 1793-1805, doi: 10.1038/s41593-019-0498-9 (2019).
16. Puttonen, K. A. et al. Generation of Functional Neuromuscular Junctions from Human Pluripotent Stem Cell Lines. Front Cell Neurosci 9, 473, doi: 10.3389/fncel.2015.00473 (2015).
17. Faustino Martins, J. M. et al. Self-Organizing 3D Human Trunk Neuromuscular Organoids. Cell Stem Cell 26, 172-186 e176, doi: 10.1016/j.stem.2019.12.007 (2020).
18. Chal, J. & Pourquie, O. Making muscle: skeletal myogenesis in vivo and in vitro. Development 144, 2104-2122, doi: 10.1242/dev.151035 (2017).
19. Xi, H. et al. In Vivo Human Somitogenesis Guides Somite Development from hPSCs. Cel Rep 18, 1573-1585, doi: 10.1016/j.celrep.2017.01.040 (2017).
20. Chambers, S. M. et al. Highly efficient neural conversion of human ES and iPS cells by 620 dual inhibition of SMAD signaling. Nat Biotechnol 27, 275-280, doi: 10.1038/nbt. 1529 (2009).
21. Gouti, M. et al. In vitro generation of neuromesodermal progenitors reveals distinctroles for wnt signalling in the specification of spinal cord and paraxial mesoderm identity. PLOS Biol 12, e1001937, doi: 10.1371/journal.pbio.1001937 (2014).
22. Gouti, M. et al. A Gene Regulatory Network Balances Neural and Mesoderm Specification during Vertebrate Trunk Development. Dev Cell 41, 243-261 e247, doi: 10.1016/j.devcel.2017.04.002 (2017).
23. Larocca, C. et al. An autocrine loop between TGF-beta1 and the transcription factorbrachyury controls the transition of human carcinoma cells into a mesenchymal phenotype. Mol Cancer Ther 12, 1805-1815, doi: 10.1158/1535-7163.MCT-12-1007 (2013).
24. Rodrigo Albors, A., Halley, P. A. & Storey, K. G. Lineage tracing of axial progenitors using Nkx1-2CreER (T2) mice defines their trunk and tail contributions. Development 145, doi: 10.1242/dev.164319 (2018).
25. Gouti, M., Metzis, V. & Briscoe, J. The route to spinal cord cell types: a tale of signals and switches. Trends Genet 31, 282-289, doi: 10.1016/j.tig.2015.03.001 (2015).
26. Wind, M. et al. Defining the signalling determinants of a posterior ventral spinal cord identity in human neuromesodermal progenitor derivatives. Development 148, doi: 10.1242/dev.194415 (2021).
27. Dasen, J. S., Liu, J. P. & Jessell, T. M. Motor neuron columnar fate imposed by sequential phases of Hox-c activity. Nature 425, 926-933, doi: 10.1038/nature02051 (2003).
28. Amoroso, M. W. et al. Accelerated high-yield generation of limb-innervating motor neurons from human stem cells. J Neurosci 33, 574-586, doi: 10.1523/JNEUROSCI.0906-12.2013 (2013).
29. Tchieu, J. et al. NFIA is a gliogenic switch enabling rapid derivation of functional human astrocytes from pluripotent stem cells. Nat Biotechnol 37, 267-275, doi: 10.1038/s41587-019-0035-0 (2019).
30. Clovis, Y. M. et al. Chx10 Consolidates V2a Interneuron Identity through Two Distinct Gene Repression Modes. Cell Rep 16, 1642-1652, doi: 10.1016/j.celrep.2016.06.100 (2016).
31. Pillai, A., Mansouri, A., Behringer, R., Westphal, H. & Goulding, M. Lhx1 and Lhx5 maintain the inhibitory-neurotransmitter status of interneurons in the dorsal spinal cord. Development 134, 357-366, doi: 10.1242/dev.02717 (2007).
32. Mao, Q. et al. Tension-driven multi-scale self-organisation in human iPSC-derived muscle fibers. Elife 11, doi: 10.7554/eLife.76649 (2022).
33. Mauro, A. Satellite cell of skeletal muscle fibers. J Biophys Biochem Cytol 9, 493-495, doi: 10.1083/jcb.9.2.493 (1961).
34. Frith, T. J. et al. Human axial progenitors generate trunk neural crest cells in vitro. Elife 7, doi: 10.7554/eLife.35786 (2018).
35. Lefebvre, S. et al. Identification and characterization of a spinal muscular atrophy-determining gene. Cell 80, 155-165, doi: 10.1016/0092-8674 (95) 90460-3 (1995).
36. Lorson, C. L., Hahnen, E., Androphy, E. J. & Wirth, B. A single nucleotide in the SMN gene regulates splicing and is responsible for spinal muscular atrophy. Proc Natl Acad Sci USA 96, 6307-6311, doi: 10.1073/pnas.96.11.6307 (1999).
37. Mailman, M. D. et al. Molecular analysis of spinal muscular atrophy and modification of the phenotype by SMN2. Genet Med 4, 20-26, doi: 10.1097/00125817-200201000-00004 (2002).
38. Ogino, S. & Wilson, R. B. Genetic testing and risk assessment for spinal muscular atrophy (SMA). Hum Genet 111, 477-500, doi: 10.1007/s00439-002-0828-x (2002).
39. Guettier-Sigrist, S., Coupin, G., Braun, S., Warter, J. M. & Poindron, P. Muscle could be the therapeutic target in SMA treatment. J Neurosci Res 53, 663-669, doi: 10.1002/(SICI) 1097-4547 (19980915) 53: 6<663::AID-JNR4>3.0.CO; 2-3 (1998).
40. Bricceno, K. V. et al. Survival motor neuron protein deficiency impairs myotube formation by altering myogenic gene expression and focal adhesion dynamics. Hum Mol Genet 23, 4745-4757, doi: 10.1093/hmg/ddu189 (2014).
41. Hellbach, N. et al. Impaired myogenic development, differentiation and function in hESC-derived SMA myoblasts and myotubes. PLOS One 13, e0205589, doi: 10.1371/journal.pone.0205589 (2018).
42. Mutsaers, C. A. et al. Reversible molecular pathology of skeletal muscle in spinal muscular atrophy. Hum Mol Genet 20, 4334-4344, doi: 10.1093/hmg/ddr360 (2011).
43. Murray, L. M. et al. Selective vulnerability of motor neurons and dissociation of pre- and post-synaptic pathology at the neuromuscular junction in mouse models of spinal muscular atrophy. Hum Mol Genet 17, 949-962, doi: 10.1093/hmg/ddm367 (2008).
44. Kariya, S. et al. Reduced SMN protein impairs maturation of the neuromuscular junctions in mouse models of spinal muscular atrophy. Hum Mol Genet 17, 2552-2569, doi: 10.1093/hmg/ddn156 (2008).
45. Ling, K. K., Lin, M. Y., Zingg, B., Feng, Z. & Ko, C. P. Synaptic defects in the spinal and neuromuscular circuitry in a mouse model of spinal muscular atrophy. PLOS One 5, 69 e15457, doi: 10.1371/journal.pone.0015457 (2010).
46. Ling, K. K., Gibbs, R. M., Feng, Z. & Ko, C. P. Severe neuromuscular denervation of clinically relevant muscles in a mouse model of spinal muscular atrophy. Hum MolGenet 21, 185-195, doi: 10.1093/hmg/ddr453 (2012).
47. Rao, L., Qian, Y., Khodabukus, A., Ribar, T. & Bursac, N. Engineering human pluripotent stem cells into a functional skeletal muscle tissue. Nat Commun 9, 126, doi: 10.1038/s41467-017-02636-4 (2018).
48. Wang, X. et al. Genome-wide analysis of PDX1 target genes in human pancreatic progenitors. Mol Metab 9, 57-68, doi: 10.1016/j.molmet.2018.01.011 (2018).
49. Donlin-Asp, P. G. et al. The Survival of Motor Neuron Protein Acts as a Molecular Chaperone for mRNP Assembly. Cell Rep 18, 1660-1673, doi: 10.1016/j.celrep.2017.01.059 (2017).
50. Swinnen, B., Robberecht, W. The phenotypic variability of amyotrophic lateral sclerosis. Nat Rev Neurol 10, 661-670 (2014). https://doi.org/10.1038/nrneurol.2014.184

1. An in vitro method of generating a neuro-muscular junction cell culture model, comprising:
a) cultivating a first cell culture comprising pluripotent stem cells (PSCs), for at least 3 days, in a first culture medium, comprising:
a WNT pathway-activator, and
FGF,
b) replacing the first culture medium by a second culture medium, comprising:
a BMP pathway inhibitor, and (i) a TGFβ pathway inhibitor or (ii) GDF11,
Sonic Hedgehog (SHH) smoothened agonist (SAG) and/or retinoic acid (RA),
a WNT pathway-activator, and
FGF, HGF, and/or IGF1, and
cultivating the cells for at least 3 days,
thereby generating a cell culture model comprising segregated neural and mesodermal progenitor cells, and/or differentiated muscle cells with innervating motor neurons, forming neuromuscular junctions.

2. The in vitro method according to claim 1, wherein the cell culture model is a two-dimensional (2D), self-organizing neuro-muscular junction cell culture model.

3. The in vitro method according to claim 1, wherein the method further comprises step c):
replacing the at least second culture medium by a culture medium comprising:
HGF and/or IGF1, and optionally a gamma-secretase inhibitor (e.g., DAPT), and cultivating the cells for at least 4 days, and optionally replacing the culture medium by a medium suitable for maintenance of the cell culture model.

4. The in vitro method according to claim 1, wherein step b) further comprises replacing after 2-5 days the second culture medium by a third culture medium comprising retinoic acid and/or SAG, and optionally HGF and/or IGF1, and cultivating the cells for 2-5 days.

5. The in vitro method according to claim 1, wherein the method comprises the steps of:
a) Cultivating a first cell culture comprising pluripotent stem cells (PSCs; preferably iPS cells, more preferably iPSCs of a patient), for at least 3 days, in a first culture medium, comprising:
a WNT pathway-activator, and
FGF,
b) replacing the first culture medium by a second culture medium, comprising one or more of the group comprising a WNT pathway-activator, an FGF, SHH smoothened agonist (SAG), a BMP pathway inhibitor, and either (i) a TGFβ pathway inhibitor and retinoic acid (RA), or (ii) GDF11, and cultivating the cells for 2-4 days,
c) replacing the second culture medium by a third culture medium comprising retinoic acid, SAG, HGF and IGF1, and cultivating the cells for 2-5 days, wherein on the first day a Rho-associated protein kinase inhibitor (ROCKi) is added to the culture medium for 6-24 hours,
d) replacing the third culture medium by a fourth culture medium comprising HGF and IGF1, and optionally a gamma-secretase inhibitor, and cultivating the cells for 1-5 days,
e) replacing the fourth culture medium by a fifth culture medium comprising HGF and IGF1, and cultivating the cells for at least 11 days,
f) replacing the fifth culture medium by a sixth culture medium comprising a ROCK inhibitor, ITS (Insulin, Transferrin, Selenium), HGF and IGF1, and cultivating the cells for 1-4 days, and
g) replacing the sixth culture medium by a seventh culture medium supplemented with ITS and cultivating the cells for at least one day.

6. The in vitro method according to claim 1, wherein after 3 days of total culture duration the generation of brachial neurons and optionally cervical and/or thoracic neurons, is supported by adding a BMP pathway inhibitor, a TGFβ pathway inhibitor and optionally retinoic acid (RA).

7. The in vitro method according to claim 1, wherein after 3 days of total culture duration the generation of lumbar neurons is supported by adding a BMP pathway inhibitor and GDF11.

8. The in vitro method according to claim 1, wherein after 6 days of total culture duration at least a fraction of the neural progenitor cells expresses SOX2 and at least a fraction of the mesodermal progenitor cells expresses PAX3, and wherein the cells segregate into neural and mesodermal progenitor clusters.

9. The in vitro method according to claim 1, wherein after 20 days of total culture duration at least a fraction of the neural progenitor cells are brachial neurons and optionally cervical and/or thoracic neurons, expressing one or more of HOXC6, HOXC5, HOXC8, HOXC9, ISL1, CHAT and TUBB3.

10. The in vitro method according to claim 1, wherein after 20 days of total culture duration, at least a fraction of the neural progenitor cells are lumbar neurons expressing one or more of HOXC10, HOXC9, ISL1, CHAT and TUBB3.

11. The in vitro method according to claim 1, wherein:
a. after 20 days of total culture duration at least a fraction of the neural progenitor cells expresses one or more of the factors of TUBB3, ISL1 and CHAT, and the muscle progenitor cells express one or more of the factors of DESMIN, MYOD and MYF5, and/or
b. wherein after 50 days of total culture duration at least a fraction of the neurons express TUBB3 and at least a fraction of the muscle cells express MSKF, DESMIN, and/or TITIN.

12. The in vitro method according to claim 1, wherein the first cell culture comprises pluripotent stem cells of a patient, and wherein the patient has been diagnosed with and/or is suffering from a myopathy, motor-neuron disease, an auto-immune neuromuscular disease, or other disease affecting the muscular or neuromuscular system of said patient.

13. An in vitro cell culture model produced according to the method of claim 1, comprising (i) segregated neural and mesodermal progenitor cells, (ii) segregated partially differentiated neural and myoblast cells, (iii) brachial, and optionally cervical and/or thoracic, motor neurons, or (iv) lumbar motor neurons, wherein the models of (iii) and/or (iv) comprise bundles of muscle fibers surrounded by innervating motor neurons forming neuromuscular junctions.

14. An in vitro cell culture model, comprising (i) segregated neural and mesodermal progenitor cells, (ii) segregated partially differentiated neural and myoblast cells, and/or (iii) bundles of muscle fibers surrounded by innervating motor neurons, forming neuromuscular junctions.

15. An in vitro cell culture model according to claim 14, comprising segregated neural and mesodermal progenitor cells, wherein at least a fraction of the neural progenitor cells expresses SOX2, and at least a fraction of the mesodermal progenitor cells expresses PAX3, and wherein at least a fraction of the cells segregates into neural and mesodermal progenitor clusters.

16. An in vitro cell culture model according to claim 14, comprising segregated partially differentiated neural and myoblast cells, wherein at least a fraction of the neural cells expresses TUBB3, and at least a fraction of myoblast cells expresses DESMIN and form bundles of fibers that are surrounded by and/or at least partially interwoven with axonal structures of neural cells.

17. The in vitro cell culture model according to claim 16, wherein at least a fraction of the bundles of fibers has a length of over 50 μm, and at least a fraction of the axonal structures of neural cells has a length of over 20 μm.

18. An in vitro cell culture model according to claim 14, comprising neuro-muscular junctions, wherein the model comprises bundles of muscle fibers surrounded by innervating motor neurons forming functional neuromuscular junctions, wherein at least a fraction of the neurons express TUBB3, and at least a fraction of the muscle cells expresses MSKF and/or MYF5.

19. The in vitro cell culture model according to claim 18, wherein at least a fraction of the motor neurons form axons of 50-600 μm length and at least a fraction of the muscle fibers has a length of 100-3000 μm.

20. An in vitro cell culture model according to claim 14, comprising differentiated brachial, and optionally cervical and/or thoracic, motor neurons, comprising neuro-muscular junctions, wherein at least a fraction of the motor neurons expresses one or more of HOXC6, HOXC5, HOXC8, ISL1, CHAT and TUBB3.

21. An in vitro cell culture model according to claim 14, comprising differentiated lumbar motor neurons comprising neuro-muscular junctions, wherein at least a fraction of the motor neurons expresses one or more of HOXC10, HOXC9, ISL1, CHAT, ISL1 and TUBB3.

22. The in vitro cell culture model according to claim 14, wherein the cell culture model is a two-dimensional (2D), self-organizing neuro-muscular junction cell culture model.

23. The in vitro cell culture model according to claim 14, wherein the model is established from pluripotent stem cells of a patient, and wherein the patient has been diagnosed with and/or is suffering from myopathy, motor-neuron diseases, an auto-immune neuromuscular disease, or a disease affecting the muscular or neuromuscular system of said patient.

24. A method for drug screening, toxicity screening and/or for studying a disease, comprising the in vitro cell culture model according to claim 14.

25. A kit comprising a supplement-cocktail and/or cell culture medium, comprising:
a WNT pathway-activator,
an FGF,
a BMP pathway inhibitor,
(i) a TGFβ pathway inhibitor or (ii) GDF11,
A Sonic Hedgehog (SHH) smoothened agonist (SAG) and/or retinoic acid (RA), and
HGF and/or IGF1,
optionally comprising a gamma-secretase inhibitor.
